(19) United States
(12) Patent Application Publication
(10) Pub. No.: US 2019/0196326 A1
(43) Pub. Date: Jun. 27, 2019

(54) ACTINIC RAY-SENSITIVE OR RADIATION-SENSITIVE RESIN COMPOSITION, RESIST FILM, PATTERN FORMING METHOD, AND METHOD OF MANUFACTURING ELECTRONIC DEVICE

(71) Applicant: FUJIFILM Corporation, Tokyo (JP)

(72) Inventors: Masafumi KOJIMA, Shizuoka (JP); Daisuke ASAKAWA, Shizuoka (JP); Akiyoshi GOTO, Shizuoka (JP); Keita KATO, Shizuoka (JP); Keiyu OU, Shizuoka (JP)

(73) Assignee: FUJIFILM Corporation, Tokyo (JP)

(21) Appl. No.: 16/286,984

(22) Filed: Feb. 27, 2019

Related U.S. Application Data
(63) Continuation of application No. PCT/JP2017/021494, filed on Jun. 9, 2017.

(30) Foreign Application Priority Data
Aug. 31, 2016 (JP) 2016-170028
Jan. 23, 2017 (JP) 2017-009460

(51) Int. Cl. G03F 7/004; G03F 7/20; C07C 309/17; C07C 309/16; C07C 309/19; C07C 309/07; C07C 317/06; C07C 317/12; C07D 295/185; G03F 7/32

(52) U.S. Cl. CPC G03F 7/0045; G03F 7/2004; C07C 309/17; C07C 309/16; C07C 309/19; G03F 7/0046; C07C 2603/74; C07C 317/06; C07C 317/12; C07D 295/185; G03F 7/32; C07C 2601/14; C07C 309/07

(57) ABSTRACT

An actinic ray-sensitive or radiation-sensitive resin composition contains a compound that generates an acid represented by Formula (I) by irradiation with an actinic ray or radiation, and a resin. The resist film is formed of the actinic ray-sensitive or radiation-sensitive resin composition. In the pattern forming method and the method of manufacturing an electronic device, the actinic ray-sensitive or radiation-sensitive resin composition is used.

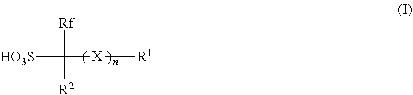

### CROSS-REFERENCE TO RELATED APPLICATIONS

This application is a Continuation of PCT International Application No. PCT/JP2017/021494 filed on Jun. 9, 2017, which claims priority under 35 U.S.C § 119(a) to Japanese Patent Application No. 2016-170028 filed on Aug. 31, 2016 and Japanese Patent Application No. 2017-009460 filed on Jan. 23, 2017. Each of the above application(s) is hereby expressly incorporated by reference, in its entirety, into the present application.

### BACKGROUND OF THE INVENTION

### 1. Field of the Invention

The present invention relates to an actinic ray-sensitive or radiation-sensitive resin composition, a resist film, a pattern forming method, and a method of manufacturing an electronic device.

### 2. Description of the Related Art

In the related art, microfabrication by lithography using a radiation-sensitive resin composition has been performed in the process of manufacturing a semiconductor device such as an integrated circuit (IC) and a large scale integrated circuit (LSI).

For example, JP5900255B discloses a radiation-sensitive resin composition containing a monosulfonic acid-type acid generator which is cleaved upon irradiation with radiation. The acid generated by cleavage of the acid generator has a function of making a deprotection reaction of the resin component in the composition occur or a crosslinking reaction of the resin component occur.

In the section of the example of JP5900255B, as described below, an acid generator having a structure in which a portion of hydrogen atoms on carbon atoms (in other words, on the carbon atoms bonded to the sulfonic acid ion) at the α-position of a sulfonic acid ion is substituted with fluorine atoms is specifically disclosed.

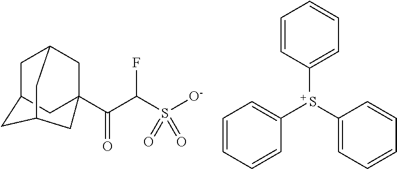

### SUMMARY OF THE INVENTION

The present inventors have conducted research on the actinic ray-sensitive or radiation-sensitive resin composition containing the acid generator specifically described in the section of the example of JP5900255B and found that, in a case where the actinic ray-sensitive or radiation-sensitive resin composition is preserved for a predetermined period of time, a temporal change such as the increase of the number of particles or the decrease of the sensitivity easily occurs. That is, the present inventors have found that it is necessary to further improve the preservation stability.

The present inventors also have found that a resist pattern forming by the actinic ray-sensitive or radiation-sensitive resin composition containing the acid generator needs to be further improved even in the pattern line width roughness (LWR).

Accordingly, an object of the present invention is to provide an actinic ray-sensitive or radiation-sensitive resin composition which has excellent preservation stability and has small pattern line width roughness (LWR) in a case where a resist pattern is formed.

Another object of the present invention is to provide a resist film, a pattern forming method, and a method of manufacturing an electronic device, each of which uses the actinic ray-sensitive or radiation-sensitive resin composition.

As a result of diligent research so as to achieve the above object, the present inventors have found that the aforementioned objects can be achieved by causing the actinic ray-sensitive or radiation-sensitive resin composition to contain a compound which generates an acid having a specific structure so as to complete the present invention.

That is, the present inventors have found that the above objects can be achieved by the following configurations.

(1) An actinic ray-sensitive or radiation-sensitive resin composition comprising: a compound that generates an acid represented by Formula (I) by irradiation with an actinic ray or radiation; and a resin.

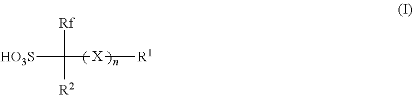

(2) The actinic ray-sensitive or radiation-sensitive resin composition according to (1), wherein, in Formula (I), R¹ represents a hydrocarbon group having 1 to 20 carbon atoms.

(3) The actinic ray-sensitive or radiation-sensitive resin composition according to (1) or (2), wherein, in Formula (I), R² represents a hydrocarbon group having 2 to 20 carbon atoms which may include a hetero atom.

(4) The actinic ray-sensitive or radiation-sensitive resin composition according to any one of (1) to (3), wherein, in Formula (I), R¹ is a linear or branched alkyl group, and R² is an alkyl group having 2 to 20 carbon atoms.

(5) The actinic ray-sensitive or radiation-sensitive resin composition according to any one of (1) to (4), in Formula (I), n is 1.

(6) The actinic ray-sensitive or radiation-sensitive resin composition according to any one of (1) to (5), wherein the resin is a resin that is decomposed due to an action of an acid to increase polarity.

(7) A resist film that is formed of the actinic ray-sensitive or radiation-sensitive resin composition according to any one of (1) to (6).

(8) A pattern forming method comprising:

forming a resist film by using the actinic ray-sensitive or radiation-sensitive resin composition according to any one of (1) to (6);

exposing the resist film; and

developing the exposed resist film with a developer.

(9) The pattern forming method according to (8), wherein the developer contains an organic solvent.

(10) A method of manufacturing an electronic device, comprising: the pattern forming method according to (8) or (9).

According to the present invention, it is possible to provide an actinic ray-sensitive or radiation-sensitive resin composition having excellent preservation stability and having small pattern line width roughness (LWR) in a case where a resist pattern is formed.

According to the present invention, it is possible to provide a resist film, a pattern forming method, and a method of manufacturing an electronic device, each of which uses the actinic ray-sensitive or radiation-sensitive resin composition.

### DESCRIPTION OF THE PREFERRED EMBODIMENTS

Hereinafter, the present invention is specifically described.

The following description of constituent elements may be made based on a representative embodiment of the present invention, but the present invention is not limited to the embodiment.

In the present specification, in a case where there is no description regarding whether a group (atomic group) is substituted or unsubstituted, the group includes both of a group having a substituent and a group not having a substituent. For example, an “alkyl group” includes not only an alkyl group not having a substituent (unsubstituted alkyl group) but also an alkyl group having a substituent (substituted alkyl group).

An “actinic ray” or a “radiation” in the present specification, for example, means a bright line spectrum of a mercury lamp, a far ultraviolet ray represented by an excimer laser, an extreme ultraviolet ray (EUV ray), an X-ray, and an electron beam (EB). In the present invention, the light means actinic rays or radiation.

Unless described otherwise, the “exposure” in the present specification include not only exposure to a bright line spectrum of a mercury lamp, a far ultraviolet ray represented by an excimer laser, an extreme ultraviolet ray (EUV), and an X-ray but also drawing by a particle ray such as an electron beam and an ion beam.

In the present specification, “to” is used to mean that the numerical values listed before and after “to” are a lower limit and an upper limit respectively.

In the present specification, the weight-average molecular weight (Mw) and the number-average molecular weight (Mn) are values in terms of polystyrene obtained by gel permeation chromatography (GPC) with tetrahydrofuran (THF) as a development solvent.

In the present specification, “(meth)acrylic acid” means both of “acrylic acid and methacrylic acid”.

### Actinic Ray-Sensitive or Radiation-Sensitive Resin Composition

The actinic ray-sensitive or radiation-sensitive resin composition according to the embodiment of the present invention contains a compound (hereinafter, simply referred to as an “acid generator”) that generates an acid represented by Formula (I) by the irradiation with an actinic ray or radiation and a resin.

The actinic ray-sensitive or radiation-sensitive resin composition according to the embodiment of the present invention has the above configuration and thus has excellent preservation stability and small pattern line width roughness (LWR) in a case where a resist pattern is formed.

The reason is not clear, but it is assumed as follows.

In the compound that generates an acid represented by Formula (I) due to the irradiation of an actinic ray or radiation described below, all hydrogen atoms on carbon atoms at an α-position of a sulfonic acid ion are substituted. Particularly, the compound includes an organic group having 1 or more carbon atoms as R¹ and an organic group having 2 or more carbon atoms as R² are respectively included.

The acid generator specifically indicated in the section of the examples of JP5900255B has a structure in which hydrogen atoms on carbon atoms at an α-position of a sulfonic acid ion are interposed between a sulfonic acid ion, an electron withdrawing group (carbonyl group or alkoxycarbonyl group), and a fluorine atom. Due to this structural factor, the hydrogen atom is in a state of being easily drawn out by the basic compound. That is, the acid generator specifically disclosed in the section of the example of JP5900255B is easily decomposed by drawing out the hydrogen atoms, and thus an actinic ray-sensitive or radiation-sensitive resin composition containing the acid generator has inferior preservation stability.

Meanwhile, in the compound which generates an acid represented by Formula (I) upon irradiation with an actinic ray or radiation described below, compared with a case where a compound does not have a hydrogen atom on a carbon atom at an α-position of a sulfonic acid ion, decomposition like under preservation by a basic compound or the is suppressed. As a result, it is assumed that the actinic ray-sensitive or radiation-sensitive resin composition containing the acid generator has excellent preservation stability and particularly the increase of the number of particles or the decrease of sensitivity after temporal preservation is suppressed.

In the compound that generates an acid represented by Formula (I) due to the irradiation with an actinic ray or radiation described below has a structure in which all hydrogen atoms on carbon atoms at an α-position of a sulfonic acid ion are substituted, and thus an edge part, of the sulfonic acid is bulky.

It is assumed that, with respect to the acid represented by Formula (I), diffusibility is suppressed due to the aforementioned structural characteristics, and thus invasion into the non-exposed portion can be reduced. As a result, it is considered that a resist pattern having small pattern line width roughness (LWR) can be obtained.

Hereinafter, components included in the actinic ray-sensitive or radiation-sensitive resin composition (hereinafter, also referred to as a “composition according to the embodiment of the present invention”) according to the embodiment of the present invention are described.

### Acid Generator

The acid generator included in the composition according to the embodiment of the present invention generates an acid represented by Formula (I) due to the irradiation of an actinic ray or radiation.

The acid generator may have an aspect of a low molecular weight compound or may have an aspect of a polymer.

In a case where the acid generator is in the aspect of a low molecular weight compound, the molecular weight is preferably 3000 or less, more preferably 2000 or less, and even more preferably 1000 or less.

In a case where the acid generator has an aspect of a polymer, the structure thereof is not particularly limited, and for example, may be incorporated into a portion of a <resin (A)> described below. In a case where the acid generator has an aspect of a polymer, the weight-average molecular weight thereof is preferably 1,000 to 200,000 and more preferably 2,000 to 20,000 as a value in terms of polystyrene by a GPC method.

The acid represented by Formula (I) is described below.

(Acid represented by Formula (I))

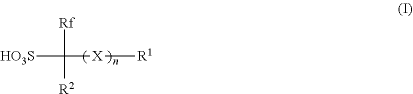

In Formula (I),

R¹ represents an organic group having 1 or more carbon atoms.

R² represents an organic group having 2 or more carbon atoms.

Rf represents a fluorine atom or a monovalent organic group including a fluorine atom.

X represents a divalent electron withdrawing group.

n represents 0 or 1.

The organic group having 1 or more carbon atoms represented by R¹ is not particularly limited, and examples thereof include hydrocarbon group having 1 to 20 carbon atoms, which may include a hetero atom. Examples of the hydrocarbon group having 1 to 20 carbon atoms, which may include a hetero atom include a hydrocarbon group having 1 to 20 carbon atoms or hydrocarbon group having 1 to 20 carbon atoms in total, which has a group combined with one selected from the group consisting of —O—, —S—, —CO—, —SO₂—, and —NR^{a}— or a plurality of these.

R^{a} represents a hydrogen atom or hydrocarbon group having 1 to 20 carbon atoms (preferably an alkyl group having 1 to 5 carbon atoms).

Examples of the hydrocarbon group having 1 to 20 carbon atoms include an alkyl group having 1 to 20 carbon atoms and aromatic hydrocarbon group having 6 to 20 carbon atoms. These groups may have a substituent.

The alkyl group having 1 to 20 carbon atoms may have any one of a linear shape, a branched shape, and a cyclic shape, and examples thereof include a methyl group, an ethyl group, a propyl group, a butyl group, a pentyl group, a hexyl group, a heptyl group, an octyl group, a nonyl group, a decyl group, a dodecyl group, a tridecyl group, a tetradecyl group, a pentadecyl group, a hexadecyl group, a heptadecyl group, an octadecyl group, a nonadecyl group, eicosyl group, a cyclopropyl group, a cyclobutyl group, a cyclopentyl group, a cyclohexyl group, a norbornyl group, and an adamantyl group.

Examples of the aromatic hydrocarbon group having 6 to 20 carbon atoms include a phenyl group.

Examples of the hydrocarbon group having 1 to 20 carbon atoms in total, which has any one selected from the group consisting of —O—, —S—, —CO—, —SO₂—, and —NR^{a}— or a group obtained by combining a plurality of these include a hydrocarbon group in which —CH₂— in the alkyl group having 1 to 20 carbon atoms is substituted with any one selected from the group consisting of —O—, —S—, —CO—, —SO₂—, and —NR^{a}—, or a group obtained by combining a plurality of these. Among these, a group in which —CH₂— in an alkyl group having 1 to 20 carbon atoms is substituted with any one selected from the group consisting of —O—, —CO—, —OCO—, and —COO— is preferable, and an alkoxyalkyl group having 2 to 20 carbon atoms, an acylalkyl group having 2 to 20 carbon atoms, or an alkyloxycarbonylalkyl group having 3 to 20 carbon atoms is more preferable.

The alkoxyalkyl group having 2 to 20 carbon atoms is preferably an alkoxyalkyl group having 2 to 10 carbon atoms, and examples thereof include a methoxyethyl group.

The acylalkyl group having 2 to 20 carbon atoms is preferably an acylalkyl group having 2 to 10 carbon atoms, and examples thereof include an acetylmethyl group and an acetylethyl group.

The alkyloxycarbonylalkyl group having 3 to 20 carbon atoms is preferably an alkyloxycarbonylalkyl group having 3 to 10 carbon atoms, and examples thereof include a methoxycarbonylmethyl group.

Among the aforementioned groups, the organic group having 1 or more carbon atoms which is represented by R¹ is preferably a hydrocarbon group having 1 to 20 carbon atoms. In view of LWR and excellent preservation stability, a linear or branched alkyl group is preferable, a linear or branched alkyl group having 1 to 5 carbon atoms is more preferable, and a linear or branched alkyl group having 1 to 3 carbon atoms is even more preferable.

The organic group having 2 or more carbon atoms which is represented by R² is not particularly limited, but examples thereof include a hydrocarbon group having 2 to 20 carbon atoms which may include a hetero atom. Examples of the hydrocarbon group having 2 to 20 carbon atoms that may include a hetero atom include a hydrocarbon group having 2 to 20 carbon atoms or a hydrocarbon group having 2 to 20 carbon atoms in total, which has any one selected from the group consisting of —O—, —S—, —CO—, —SO₂—, and —NR^{a}— or a group obtained by combining a plurality of these.

R^{a} represents a hydrogen atom or a hydrocarbon group having 1 to 20 carbon atoms (an alkyl group having 1 to 5 carbon atoms is preferable).

Examples of the hydrocarbon group having 2 to 20 carbon atoms include an alkyl group having 2 to 20 carbon atoms and an aromatic hydrocarbon group having 6 to 20 carbon atoms. These groups may have a substituent.

The alkyl group having 2 to 20 carbon atoms may have any one of a linear shape, a branched shape, and a cyclic shape, and examples thereof include an ethyl group, a propyl group, a butyl group, a pentyl group, a hexyl group, a heptyl group, an octyl group, a nonyl group, a decyl group, a dodecyl group, a tridecyl group, a tetradecyl group, a pentadecyl group, a hexadecyl group, a heptadecyl group, an octadecyl group, a nonadecyl group, an eicosyl group, a cyclopropyl group, a cyclobutyl group, a cyclopentyl group, a cyclohexyl group, a norbornyl group, and an adamantyl group.

Examples of the aromatic hydrocarbon group having 6 to 20 carbon atoms include a phenyl group.

Examples of the hydrocarbon group having 2 to 20 carbon atoms in total, which has any one selected from the group consisting of —O—, —S—, —CO—, —SO₂—, and —NR^{a}— or a group obtained by combining a plurality of these include a group in which —CH₂— in the alkyl group having 2 to 20 carbon atoms is substituted with any one selected from the group consisting of —O—, —S—, —CO—, —SO₂—, and —NR^{a}— or a group obtained by combining a plurality of these. Among these, a group in which —CH₂— in the alkyl group having 2 to 20 carbon atoms is substituted with any one selected from the group consisting of —O—, —CO—, —OCO—, and —COO— is preferable, and an alkoxyalkyl group having 2 to 20 carbon atoms, an acylalkyl group having 2 to 20 carbon atoms, or an alkyloxycarbonylalkyl group having 3 to 20 carbon atoms is more preferable.

The alkoxyalkyl group having 2 to 20 carbon atoms is preferably an alkoxyalkyl group having 2 to 10 carbon atoms, and examples thereof include a methoxyethyl group.

The acylalkyl group having 2 to 20 carbon atoms is preferably an acylalkyl group having 2 to 10 carbon atoms, and examples thereof include an acetylmethyl group and an acetylethyl group.

The alkyloxycarbonylalkyl group having 3 to 20 carbon atoms is preferably an alkyloxycarbonylalkyl group having 3 to 10 carbon atoms, and examples thereof include a methoxycarbonylmethyl group.

Among the aforementioned groups, the organic group having 2 or more carbon atoms which is represented by R² is preferably a hydrocarbon group having 2 to 20 carbon atoms which may include a hetero atom, more preferably an alkyl group having 2 to 20 carbon atoms or an alkyl group having 2 to 20 carbon atoms in total in which —CH₂— is substituted with any one selected from the group consisting of —O—, —CO—, —OCO—, and —COO—, even more preferably an alkyl group having 2 to 20 carbon atoms, an alkoxyalkyl group having 2 to 20 carbon atoms, an acylalkyl group having 2 to 20 carbon atoms, or an alkyloxycarbonylalkyl group having 3 to 20 carbon atoms, and particularly preferably an alkyl group having 1 to 20 carbon atoms, an alkoxyalkyl group having 2 to 10 carbon atoms, an acylalkyl group having 2 to 10 carbon atoms, or an alkyloxycarbonylalkyl group having 3 to 10 carbon atoms.

Among these, the alkyl group having 2 to 20 carbon atoms as the organic group having 2 or more carbon atoms which is represented by R² is preferably an alkyl group having 3 to 10 carbon atoms, and more preferably an alkyl group represented by *—CH₂—X. X represents a cycloalkyl group having 3 to 9 carbon atoms or a linear alkyl group having 2 to 9 carbon atoms and preferably represents a cycloalkyl group having 3 to 9 carbon atoms. * represents a bonding position.

Examples of the monovalent organic group including a fluorine atom represented by Rf include a linear or branched alkyl group having 1 to 10 carbon atoms in which a portion or all of hydrogen atoms are substituted with a fluorine atom or a fluoroalkyl group. Specific examples thereof include CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉, and CH₂CH₂C₄F₉.

Rf is preferably a fluorine atom or a perfluoroalkyl group having 1 to 4 carbon atoms, more preferably a fluorine atom or CF₃, and even more preferably a fluorine atom.

The divalent electron withdrawing group represented by X is not particularly limited, but examples thereof include —CO—, —CON(R^{b})—, —COO—, —C(═NR^{b})—, —SO—, and —SO₂—. In the specific examples exemplified as the divalent electron withdrawing group, a bonding position thereof is not particularly limited. In Formula (I), in a case where X is —COO—, a bond formed by X and R¹ may be —OCO—R¹ or may be —COO—R¹. That is, the carbonyl carbon of —OCO— may be bonded to R¹ and the ether oxygen of —COO— may be bonded to R¹.

R^{b} represents a hydrogen atom or a hydrocarbon group (preferably an alkyl group having 1 to 5 carbon atoms) having 1 to 20 carbon atoms.

In view of acidity of the acid represented by Formula (I), X is preferably —CO— or —COO—.

In a case where X represents —CON(R^{b})—, R^{b} and R¹ may be linked to each other to form a ring.

n represents 0 or 1. In view of acidity of the acid represented by Formula (I), it is preferable that n represents 1.

Examples of the suitable aspect of the acid represented by Formula (I) include an aspect in which R¹ s a linear or branched alkyl group, R² may be a hydrocarbon group having 2 to 20 carbon atoms which may include a hetero atom, Rf is a fluorine atom, and n is 1. Among these, an aspect in which R¹ is a linear or branched alkyl group having 1 to 5 carbon atoms, R² is an alkyl group having 3 to 10 carbon atoms, Rf is a fluorine atom, and n is 1 is preferable.

One of the specific examples of the acid represented by Formula (I) is provided below.

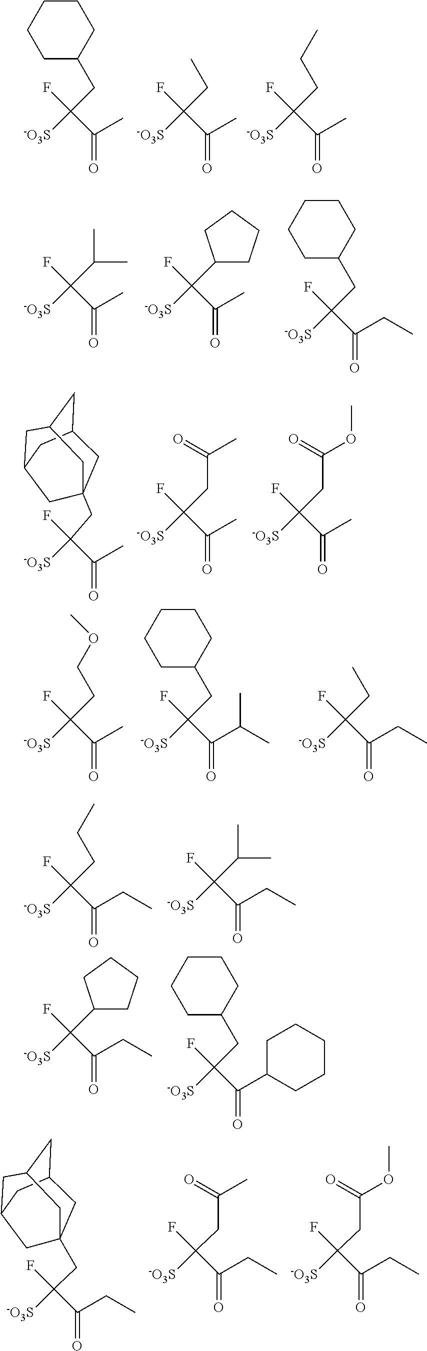

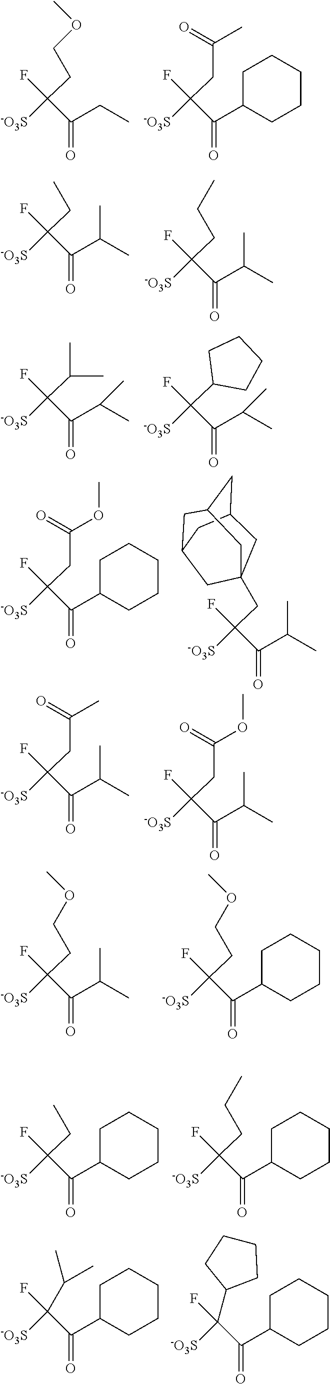

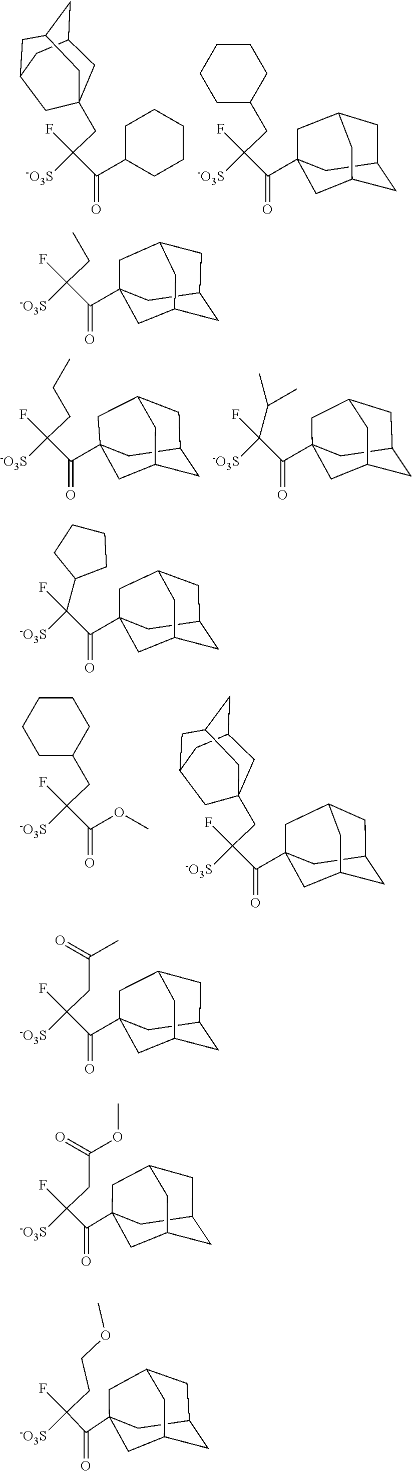

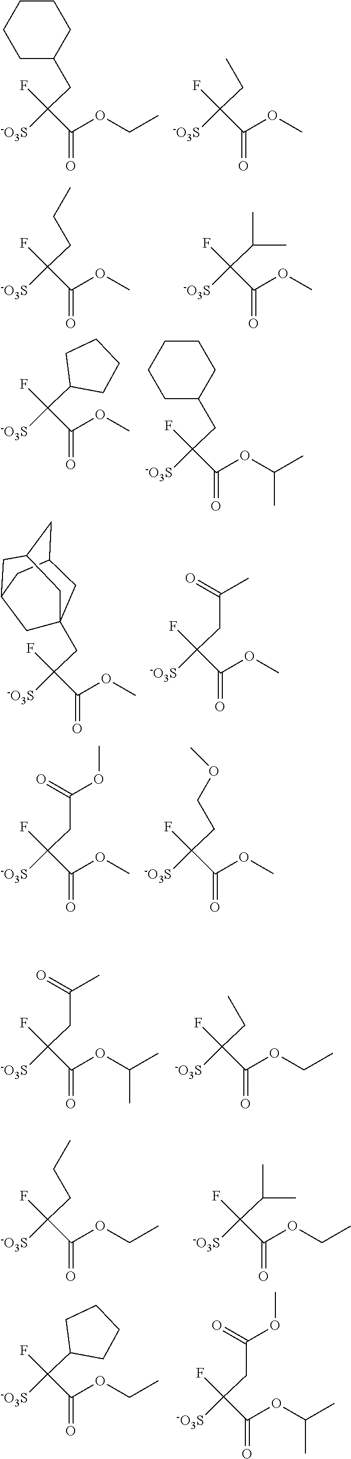

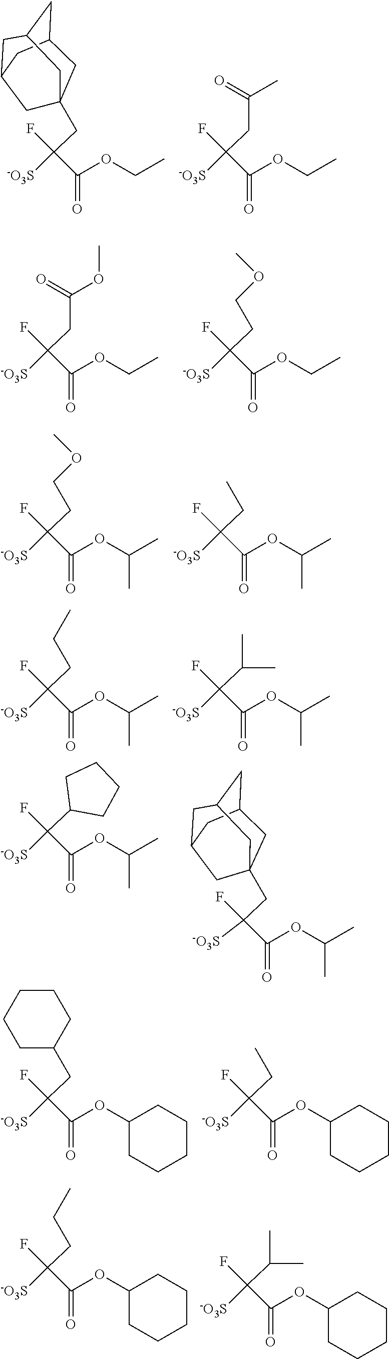

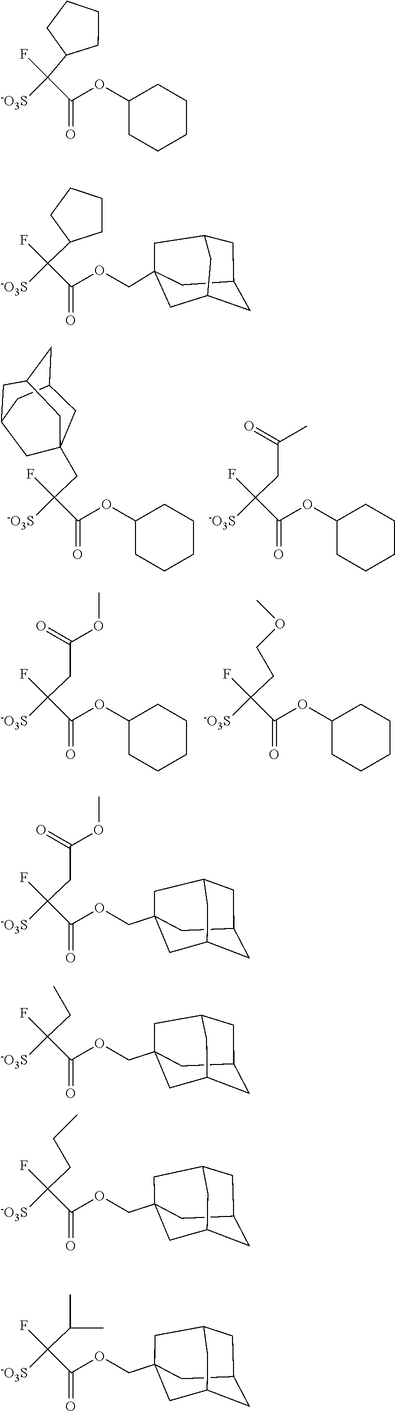

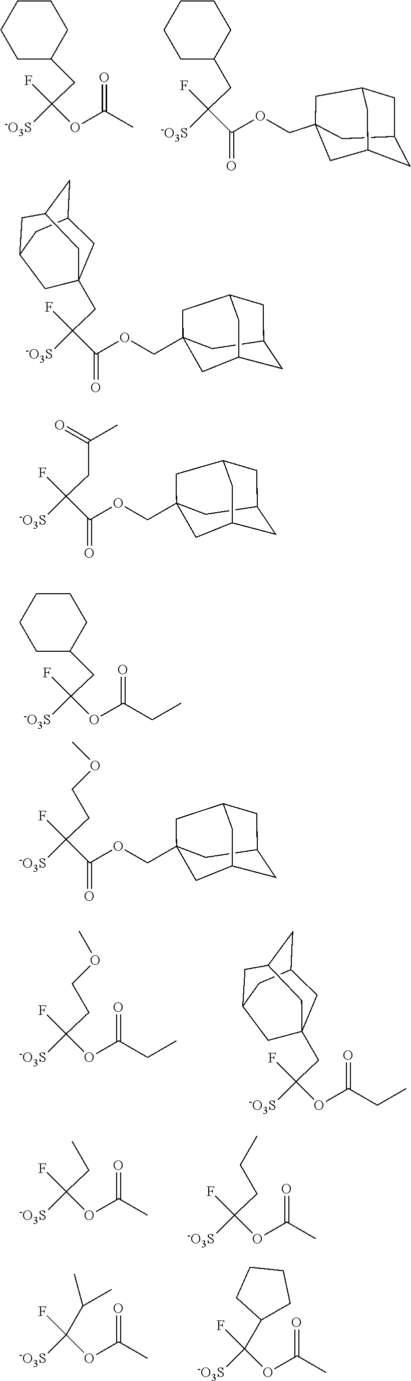

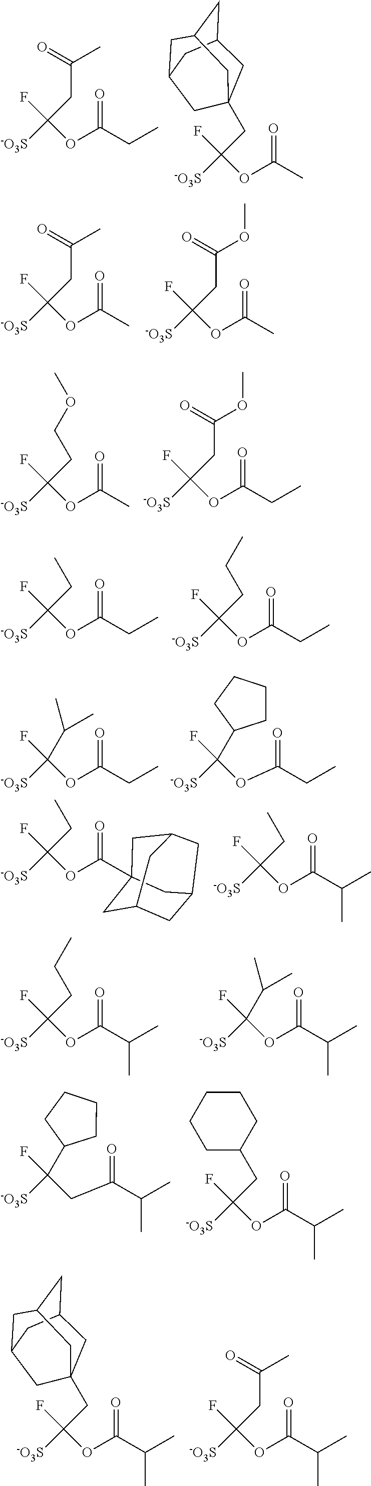

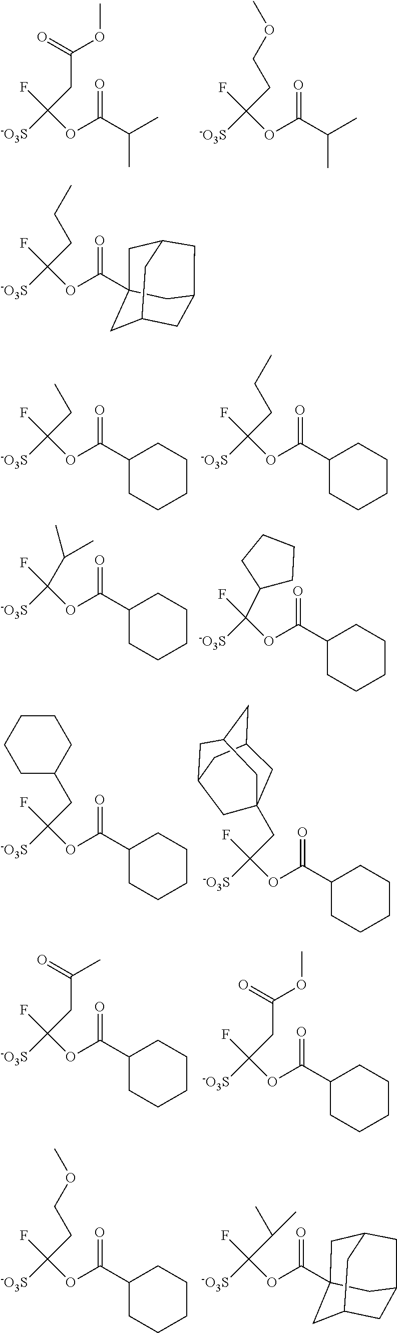

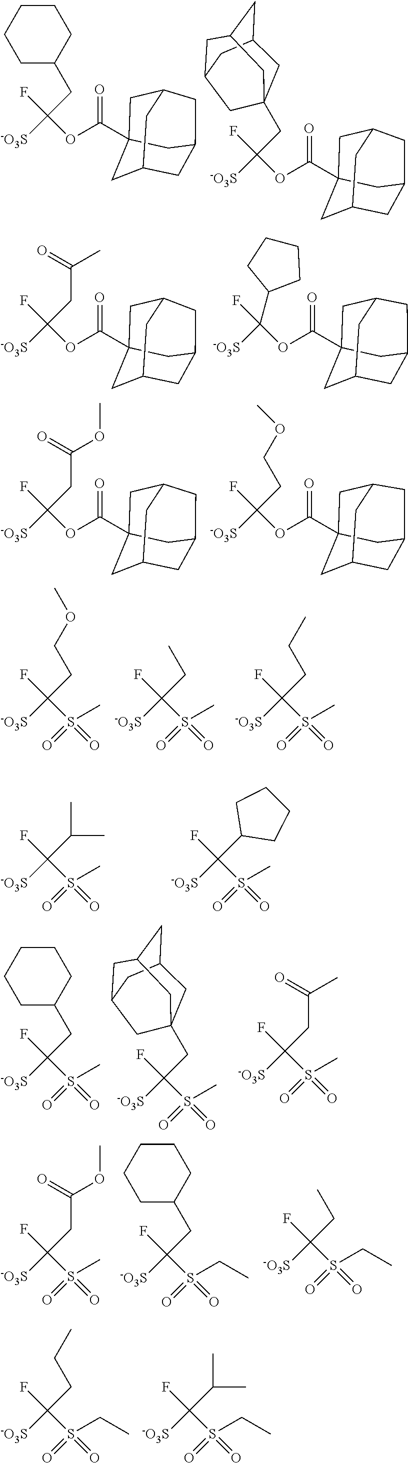

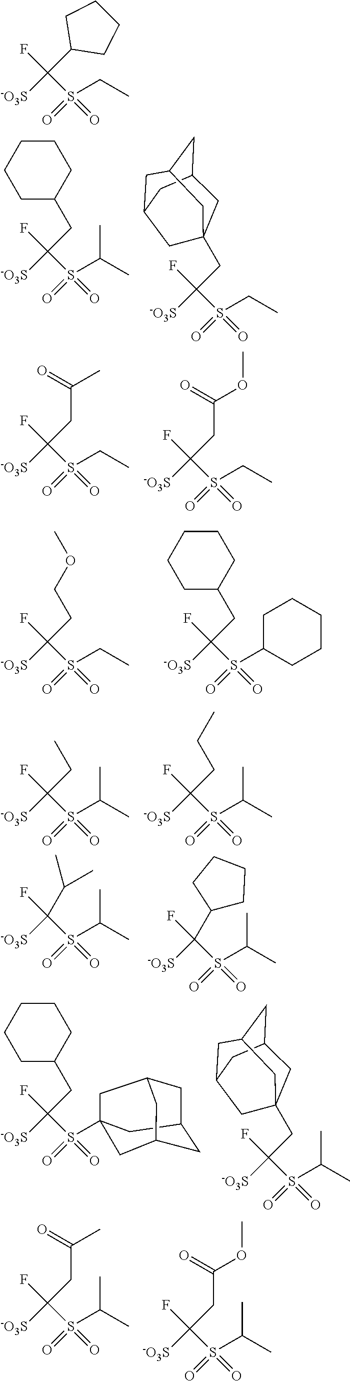

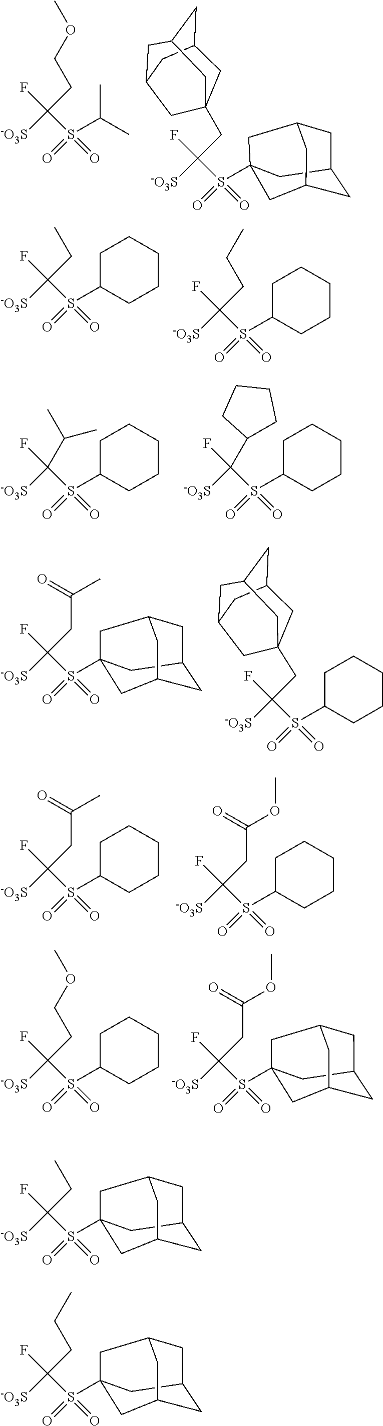

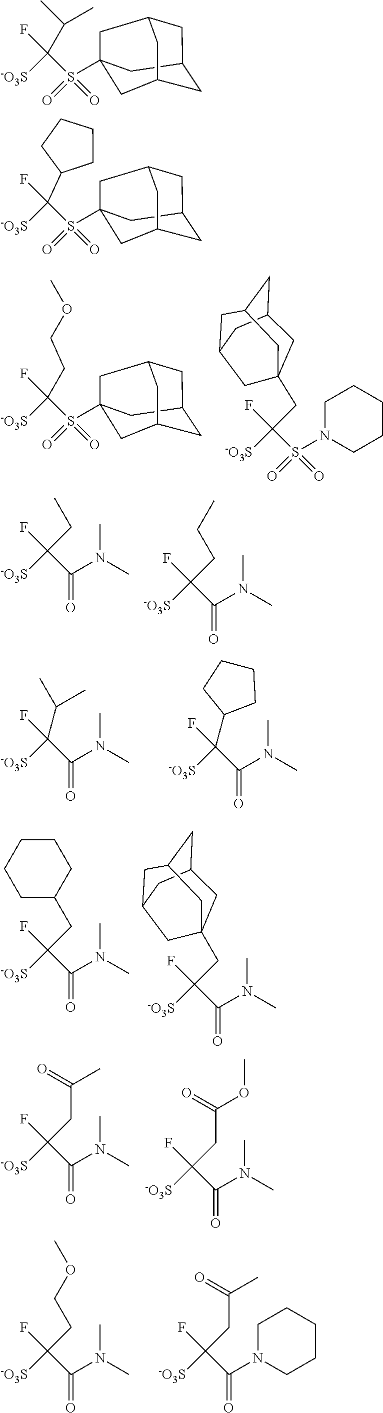

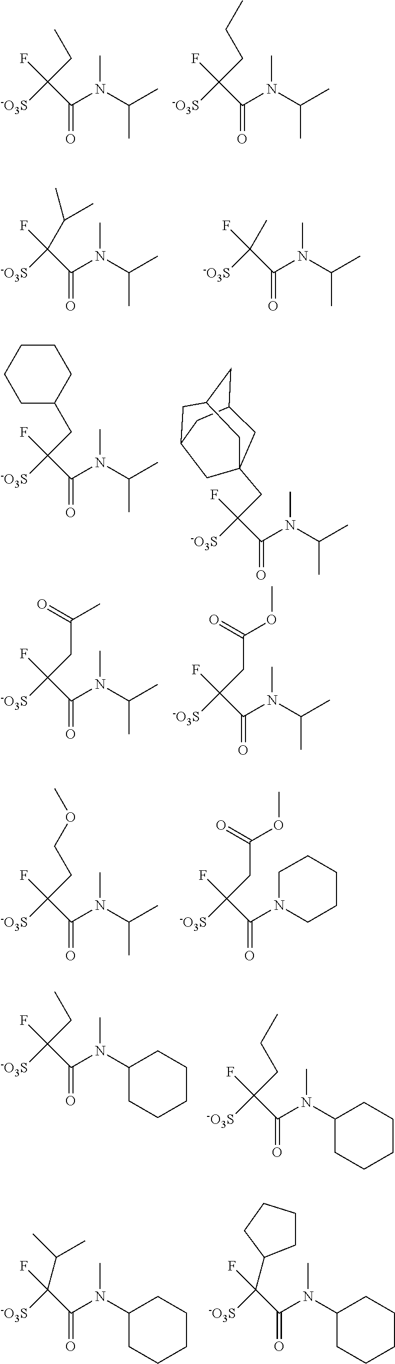

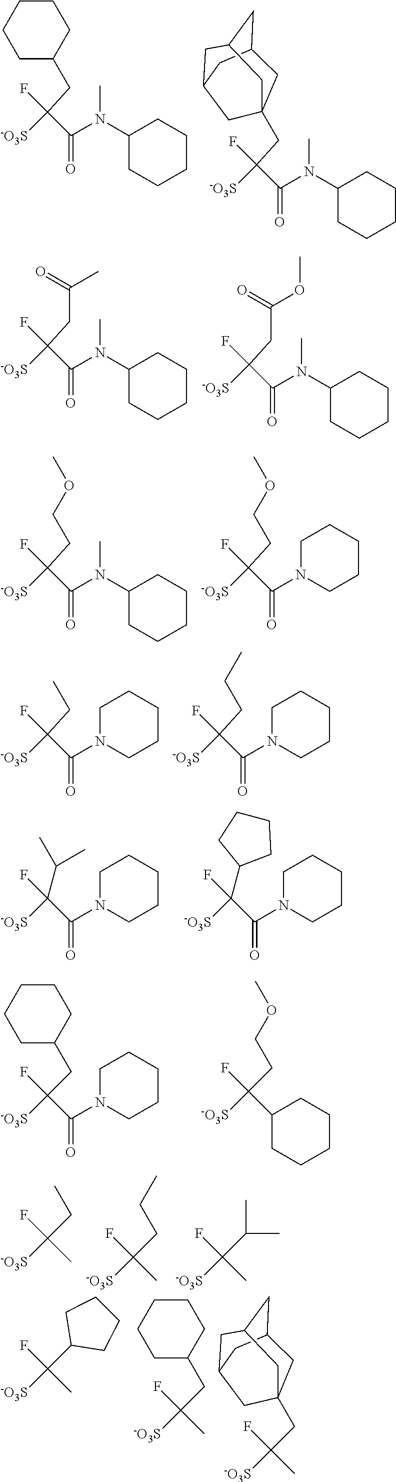

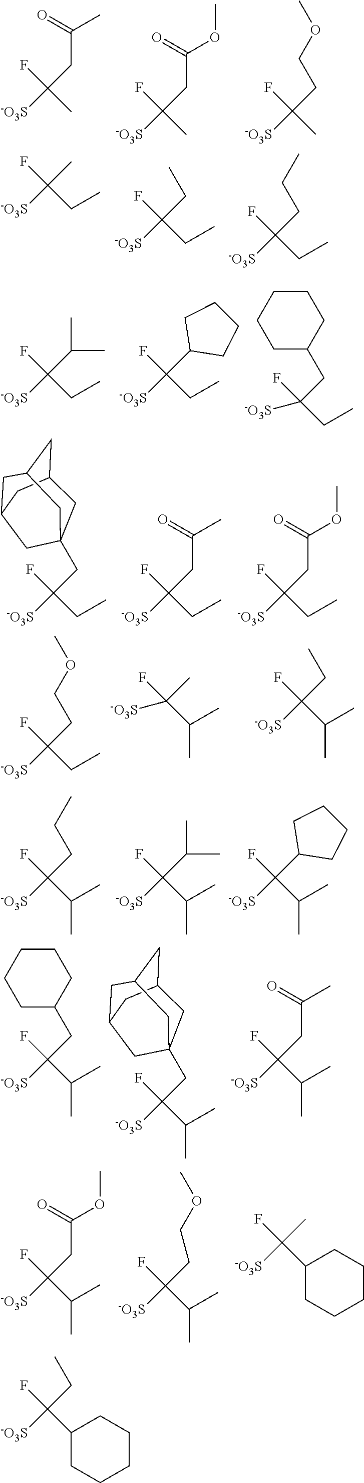

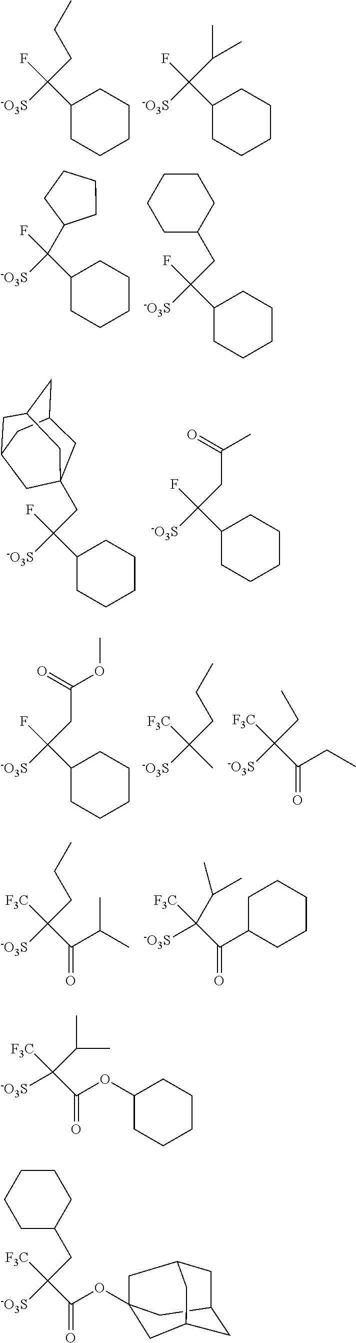

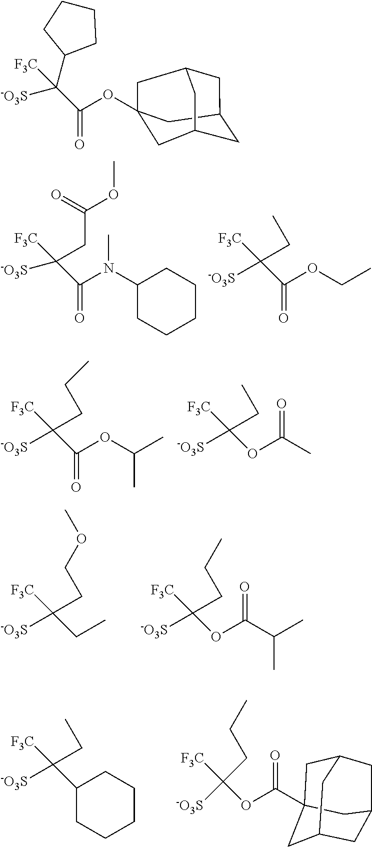

### Compound that Generates Acid Represented by Formula (I) by Irradiation of Actinic Ray or Radiation

A structure of a compound that generates an acid represented by Formula (I) due to the irradiation of an actinic ray or radiation is not particularly limited, but it is preferable to have a compound having an ionic structure of an onium salt such as a sulfonium salt and an iodonium salt or a nonionic compound structure such as oxime ester and imide ester. The onium salt is more preferably sulfonium salt.

### Compound Having Ion Structure

The compound that generates an acid represented by Formula (I) due to the irradiation of an actinic ray or radiation is preferably a compound represented by Formula (I-A).

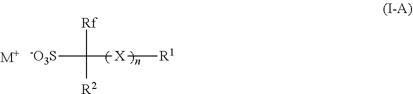

In Formula (I-A), R¹, R², Rf, X, and n have the same meaning as R¹, R², Rf, X, and n in Formula (I), and M⁺ represents a monovalent cation.

In Formula (I-A), examples of the monovalent cation represented by M⁺ include a cation represented by Formulae (ZI) and (ZII).

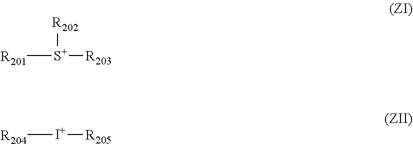

In Formula (ZI),

R₂₀₁, R₂₀₂, and R₂₀₃ each independently represent organic groups.

The number of carbon atoms of the organic group as R₂₀₁, R₂₀₂, and R₂₀₃ is generally 1 to 30 and preferably 1 to 20.

Two of R₂₀₁ to R₂₀₃ may be bonded to each other to form a ring structure and may contain an oxygen atom, a sulfur atom, an ester bond, an amide bond, or a carbonyl group in the ring. Examples of the group formed by bonding two of R₂₀₁ to R₂₀₃ include an alkylene group (for example, a butylene group and a pentylene group).

The acid generator may be a compound having a plurality of structures represented by Formula (ZI). For example, the acid generator may be a compound having a structure in which at least one of R₂₀₁, . . . , or R₂₀₃ of the compound represented by Formula (ZI) is bonded to at least one of R₂₀₁, . . . , or R₂₀₃ of another compound represented by Formula (ZI) via a single bond or a linking group.

Examples of the organic group of R₂₀₁, R₂₀₂, and R₂₀₃ include an aryl group (preferably having 6 to 15 carbon atoms), a linear or branched alkyl group (preferably having 1 to 10 carbon atoms), and a cycloalkyl group (preferably having 3 to 15 carbon atoms)

It is preferable that at least one of R_{201,} R₂₀₂, or R₂₀₃ an aryl group, and it is more preferable that all of the three are acyl groups. In addition to a phenyl group and a naphthyl group, as the aryl group, a heteroaryl group such as an indole residue, a pyrrole residue is also possible.

An aryl group, an alkyl group, and a cycloalkyl group as R₂₀₁, R₂₀₂, and R₂₀₃ each may further have a substituent. Examples the substituent include a halogen atom such as a nitro group and a fluorine atom, a carboxy group, a hydroxyl group, an amino group, a cyano group, an alkoxy group (preferably having 1 to 15 carbon atoms), a cycloalkyl group (preferably having 3 to 15 carbon atoms), an aryl group (preferably having 6 to 14 carbon atoms), an alkoxycarbonyl group (preferably having 2 to 7 carbon atoms), an acyl group (preferably having 2 to 12 carbon atoms), and an alkoxycarbonyloxy group (preferably having 2 to 7 carbon atoms), and the present invention is not limited to these.

Two selected from R₂₀₁, R₂₀₂, and R₂₀₃ may be bonded to each other via a single bond or a linking group. Examples of the linking group include an alkylene group (preferably having 1 to 3 carbon atoms), —O—, —S—, —CO— and but the present invention is not limited to these.

Examples of the preferable structure in a case where at least one of R₂₀₁, R₂₀₂, or R₂₀₃ is not an aryl group include cation structures such as compounds disclosed in paragraphs 0046 and 0047 of JP2004-233661A and paragraphs 0040 to 0046 of JP2003-035948A, compounds exemplified as Formulae (I-1) to (I-70) in US2003/0224288A1, and compounds exemplified as Formulae (IA-1) to (IA-54) and Formulae (IB-1) to (IB-24) in US2003/0077540A1.

Preferable examples of the cation represented by Formula (ZI) include cations represented by Formula (ZI-3) or (ZI-4) described below. First, a cation represented by Formula (ZI-3) is described.

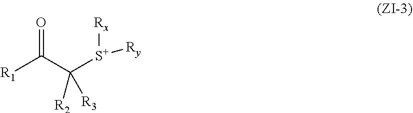

In Formula (ZI-3),

R₁ represents an alkyl group, a cycloalkyl group, an alkoxy group, a cycloalkoxy group, an aryl group, and an alkenyl group,

R₂ and R₃ each independently represent a hydrogen atom, an alkyl group, a cycloalkyl group, and an aryl group, and R₂ and R₃ may be linked to each other to form a ring,

R₁ and R₂ may be linked to each other to form a ring, and

Rₓ and R_{y} each independently represent an alkyl group, a cycloalkyl group, an alkenyl group, an aryl group, a 2-oxoalkyl group, a 2-oxocycloalkyl group, an alkoxycarbonylalkyl group, and an alkoxycarbonyl cycloalkyl group, Rₓ and R_{y} may be linked to each other to form a ring, and this ring structure may include an oxygen atom, a nitrogen atom, a sulfur atom, a ketone group, an ether bond, an ester bond, or an amide bond.

The alkyl group in R₁ is preferably a linear and branched alkyl group having 1 to 20 carbon atoms and may have an oxygen atom, a sulfur atom, or a nitrogen atom in an alkyl chain. Specific examples thereof include a linear alkyl group such as a methyl group, an ethyl group, an n-propyl group, an n-butyl group, an n-pentyl group, an n-hexyl group, an n-octyl group, an n-dodecylgroup, an n-tetradecyl group, and an n-octadecyl group, and a branched chain alkyl group such as an isopropyl group, an isobutyl group, a t-butyl group, a neopentyl group, and a 2-ethylhexyl group. The alkyl group of R₁ may have a substituent, and examples of the alkyl group having a substituent include a cyanomethyl group, a 2,2,2-trifluoroethyl group, a methoxycarbonylmethyl group, and an ethoxycarbonylmethyl group.

The cycloalkyl group as R₁ is preferably a cycloalkyl group having 3 to 20 carbon atoms and may have an oxygen atom, or a sulfur atom in the ring. Specific examples thereof include a cyclopropyl group, a cyclopentyl group, a cyclohexyl group, a norbornyl group, and an adamantyl group. The cycloalkyl group of R₁ may have a substituent, and examples of the substituent include an alkyl group and an alkoxy group.

The alkoxy group as R₁is preferably an alkoxy group having 1 to 20 carbon atoms. Specific examples thereof include a methoxy group, an ethoxy group, an isopropyloxy group, a t-butyloxy group, a t-amyloxy group, and an n-butyloxy group. The alkoxy group of R₁ may have a substituent, and examples of the substituent include an alkyl group and a cycloalkyl group.

The cycloalkoxy group as R₁ is preferably a cycloalkoxy group having 3 to 20 carbon atoms, and examples thereof include a cyclohexyloxy group, a norbornyloxy group, and an adamantyloxy group. The cycloalkoxy group of R₁ may have a substituent, and examples of the substituent include an alkyl group and a cycloalkyl group.

The aryl group as R₁ is preferably an aryl group having 6 to 14 carbon atoms, and examples thereof include a phenyl group, a naphthyl group, and a biphenyl group. The aryl group of R₁ may have a substituent, and preferable examples of the substituent include an alkyl group, a cycloalkyl group, an alkoxy group, a cycloalkoxy group, an aryloxy group, an alkylthio group, and an arylthio group. In a case where the substituent is an alkyl group, a cycloalkyl group, an alkoxy group, or a cycloalkoxy group, examples thereof include the same groups as the alkyl group, the cycloalkyl group, the alkoxy group, and the cycloalkoxy group described above as R₁.

Examples of the alkenyl group as R₁ include a vinyl group and an allyl group.

R₂ and R₃ represent a hydrogen atom, an alkyl group, a cycloalkyl group, or an aryl group, and R₂ and R₃ may be linked to each other to form a ring. It is preferable that at least one of R₂ or R₃ represents an alkyl group, a cycloalkyl group, or an aryl group. Specific examples and preferable examples of the alkyl group, the cycloalkyl group, and the aryl group represented by R₂ and R₃ are the same as the specific examples and preferable examples described above for R₁. In a case where R₂ and R₃ are linked to each other to form a ring, a sum of carbon atoms contributing to the formation of rings included in R₂ and R₃ is preferably 4 to 7 and more preferably 4 or 5.

R₁ and R₂ are linked to each other to form a ring. In a case where R₁ and R₂ are linked to each other to form a ring, it is preferable that R₁ is an aryl group (preferably a phenyl group or a naphthyl group which may have a substituent), R₂ is an alkylene group (preferably a methylene group or an ethylene group) having 1 to 4 carbon atoms, and preferable examples of the substituent include the same substituents as the substituent described above which may be included in the aryl group as R₁. According to another aspect in a case where R₁ and R₂ are linked to each other to form a ring, it is also preferable that R₁ is a vinyl group and R₂ is an alkylene group having 1 to 4 carbon atoms.

The alkyl group represented by Rₓ and R_{y} is preferably an alkyl group having 1 to 15 carbon atoms, and examples thereof include a methyl group, an ethyl group, a propyl group, an isopropyl group, an n-butyl group, an isobutyl group, a sec-butyl group, a pentyl group, a neopentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group.

The cycloalkyl group represented by Rₓ and R_{y} is preferably a cycloalkyl group having 3 to 20 carbon atoms, and examples thereof include a cyclopropyl group, a cyclopentyl group, a cyclohexyl group, a norbornyl group, and an adamantyl group.

The alkenyl group represented by Rₓ and R_{y} is preferably an alkenyl group having 2 to 30 carbon atoms, and examples thereof include a vinyl group, an allyl group, and a styryl group.

As the aryl group represented by Rₓ and R_{y}, for example, an aryl group having 6 to 20 carbon atoms is preferable, and specific examples thereof include a phenyl group, a naphthyl group, an azulenyl group, an acenaphthylenyl group, a phenanthrenyl group, a penarenyl group, a phenanthracenyl group, a fluorenyl group, an anthracenyl group, a pyrenyl group, and a benzopyrenyl group. Among them, a phenyl group or a naphthyl group is more preferable, and a phenyl group is even more preferable.

Examples of the alkyl group moiety of the 2-oxoalkyl group and the alkoxycarbonylalkyl group represented by Rₓ and R_{y} include alkyl group moieties exemplified above as Rₓ and R_{y}.

Examples of the cycloalkyl group moiety of the 2-oxocycloalkyl group and the alkoxycarbonylcycloalkyl group represented by Rₓ and R_{y} include cycloalkyl group moieties exemplified above as Rₓ and R_{y}.

The cation represented by Formula (ZI-3) is preferably a cation represented by Formulae (ZI-3a) and (ZI-3b).

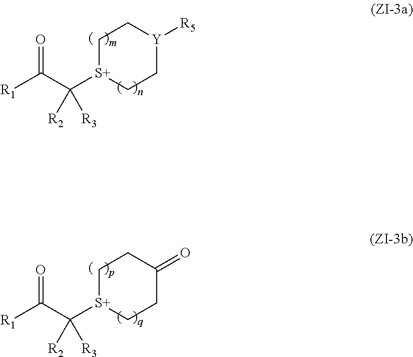

In Formulae (ZI-3a) and (ZI-3b), R₁, R₂, and R₃ are as described in Formula (ZI-3).

Y represents an oxygen atom, a sulfur atom, or a nitrogen atom and preferably an oxygen atom or a nitrogen atom, m, n, p, and q means an integer and are preferably 0 to 3, more preferably 1 to 2, and even more preferably 1. The alkylene group that links S⁺ and Y to each other may have a substituent, and preferable examples of the substituent include an alkyl group.

R₅ represents a monovalent organic group in a case where Y is a nitrogen atom and is not present in a case where Y is an oxygen atom or a sulfur atom. R₅ is preferably a group including an electron withdrawing group and particularly preferably a group represented by Formulae (ZI-3a-1) to (ZI-3a-4).

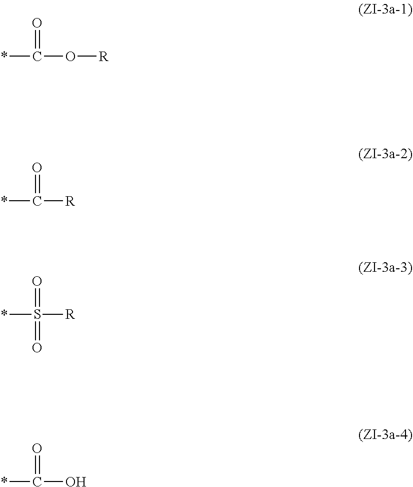

In Formulae (ZI-3a-1) to (ZI-3a-3), R represents a hydrogen atom, an alkyl group, a cycloalkyl group, or an aryl group and preferably an alkyl group. Specific examples and preferred examples of the alkyl group, the cycloalkyl group, or the aryl group for R are the same as the specific examples and preferable examples described above for R₁ in Formula (ZI-3).

In Formulae (ZI-3a-I) to (ZI-3a-4), * represents a bonding hand connected to a nitrogen atom as Y in the compound represented by Formula (ZI-3a).

In a case where Y is a nitrogen atom, R₅ is preferably a group represented by —SO₂—R₄. R₄ represents an alkyl group, a cycloalkyl group, or an aryl group, and an alkyl group is preferable. Specific examples and preferable examples of the alkyl group, the cycloalkyl group, or the aryl group for R₄ include the same examples as the specific examples and preferable examples described above for R₁.

The cation represented by Formula (ZI-3) is particularly preferably a cation represented by Formulae (ZI-3a′) and (ZI-3b′).

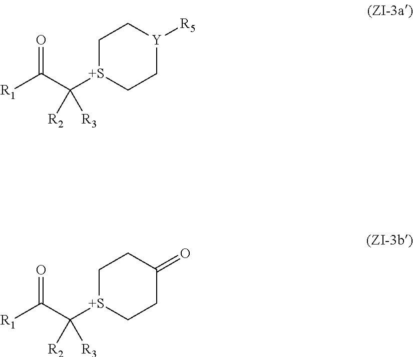

In Formulae (ZI-3a′) and (ZI-3b′), R₁, R₂, R₃, Y, and R₅ are as defined above in Formulae (ZI-3a) and (ZI-3b).

Subsequently, a cation represented by Formula (ZI-4) is described.

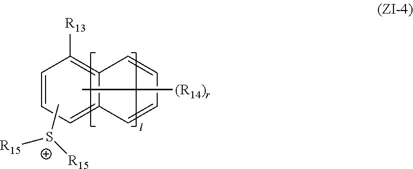

In Formula (ZI-4),

R₁₃ represents a hydrogen atom, a fluorine atom, a hydroxyl group, an alkyl group, a cycloalkyl group, an alkoxy group, an alkoxycarbonyl group, or a group having a cycloalkyl group. These groups may have a substituent.

In a case where there are a plurality of R₁₄'s, R₁₄'s each independently represent a hydroxyl group, an alkyl group, a cycloalkyl group, an alkoxy group, an alkoxycarbonyl group, an alkylcarbonyl group, an alkylsulfonyl group, a cycloalkylsulfonyl group, or a group having a cycloalkyl group. These groups may have a substituent.

R₁₅'s each independently represents an alkyl group, a cycloalkyl group, and an aryl group. Two R₁₅'s may be bonded to each other to form a ring and may include a hetero atom such as an oxygen atom, a sulfur atom, and a nitrogen atom as an atom constituting the ring. These groups may have a substituent.

l represents an integer of 0 to 2.

r represents an integer of 0 to 8.

In Formula (ZI-4), the alkyl group of R₁₃, R₁₄, and R₁₅ is linear or branched and is preferably an alkyl group having 1 to 10 carbon atoms.

Examples of the cycloalkyl groups of R₁₃, R₁₄, and R₁₅ include a monocyclic or polycyclic cycloalkyl group.

The alkoxy group of R₁₃ and R₁₄ is linear or branched and is preferably an alkoxy group having 1 to 10 carbon atoms.

The alkoxycarbonyl group of R₁₃ and R₁₄ is linear or branched and is preferably an alkoxycarbonyl group having 2 to 11 carbon atoms.

Examples of the group having the cycloalkyl groups of R₁₃ and R₁₄ include a group having a monocyclic or polycyclic cycloalkyl group. These groups may further have a substituent.

As the alkyl group of the alkylcarbonyl group of R₁₄, specific examples the same as the alkyl groups as R₁₃ to R₁₅ described above can be mentioned.

The alkylsulfonyl group and the cycloalkylsulfonyl group of R₁₄ may have be any one of a linear shape, a branched shape, and a cyclic shape and preferably have 1 to 10 carbon atoms.

Examples of the substituent that may be included in the above groups may have include a halogen atom example, a fluorine atom), a hydroxyl group, a carboxy group, a cyano group, a nitro group, an alkoxy group, an alkoxyalkyl group, an alkoxycarbonyl group, and an alkoxycarbonyloxy group.

Examples of the ring structure that may be formed by bonding two R₁₅'s to each other include a 5-membered or 6-membered ring formed by two R₁₅'s together with the sulfur atom in Formula (ZI-4), a 5-membered ring (that is, a tetrahydrothiophene ring or a 2,5-dihydrothiophene ring) is more preferable, and the ring structure may be fused with an aryl group or a cycloalkyl group. The two R₁₅'s may have a substituent, and examples of the substituent include a hydroxyl group, a carboxy group, a cyano group, a nitro group, an alkyl group, a cycloalkyl group, an alkoxy group, an alkoxyalkyl group, an alkoxycarbonyl group, and an alkoxycarbonyloxy group. A plurality of substituents for the ring structure may be present, and the substituents may be bonded to each other to form a ring.

R₁₅ in Formula (ZI-4) is preferably a methyl group, an ethyl group, an aryl group, and a divalent group in which two R₁₅'s are bonded to each other to form a tetrahydrothiophene ring structure together with the sulfur atom and more preferably a divalent group in which two R₁₅'s are bonded to each other to form a tetrahydrothiophene ring structure together with the sulfur atom.

The substituent that may be included in R₁₃ and R₁₄ is preferably a hydroxyl group, an alkoxy group, an alkoxycarbonyl group, or a halogen atom (particularly a fluorine atom).

l is preferably 0 or 1 and more preferably 1.

r is preferably 0 to 2.

Specific examples of the cation structure represented by Formula (ZI-3) or (ZI-4) described above include a cation structure such as compounds disclosed in JP2004-233661A, JP2003-035948A, US2003/0224288A1, and US2003/0077540A1 and also include cation structures in chemical structures exemplified in paragraphs 0046, 0047, 0072 to 0077, and 0107 to 0110 of JP2011-053360A and cation structures in chemical structures exemplified in paragraphs 0135 to 0137, 0151, and 0196 to 0199 of JP2011-053430A.

Subsequently, Formula (ZII) is described.

In Formulae (ZII), R₂₀₄ and R₂₀₅ each independently represent an aryl group, an alkyl group, or a cycloalkyl group.

The aryl group, the alkyl group, and the cycloalkyl group of R₂₀₁ to R₂₀₅ are the same as the aryl group, the alkyl group, and the cycloalkyl group of R₂₀₁ to R₂₀₃ in the Formula (ZI).

Among these, the aryl group of R₂₀₄ to R₂₀₅ is preferably a phenyl group or a naphthyl group and more preferably a phenyl group. The aryl groups of R₂₀₄ and R₂₀₅ may be aryl groups each having a heterocyclic structure having an oxygen atom, a nitrogen atom, a sulfur atom, or the like. Examples of the skeleton of the aryl group having a heterocyclic structure include pyrrole, furan, thiophene, indole, benzofuran, and benzothiophene.

As the alkyl group and cycloalkyl group of R₂₀₁ to R₂₀₅ a linear or branched alkyl group having 1 to 10 carbon atoms (for example, a methyl group, an ethyl group, a propyl group, a butyl group, and a pentyl group), and a cycloalkyl group having 3 to 10 carbon atoms (a cyclopentyl group, a cyclohexyl group, and a norbornyl group) are preferably provided.

An aryl group, an alkyl group, and a cycloalkyl group of R₂₀₄ to R₂₀₅ each may have a substituent. As the substituent that may be included in the aryl group, the alkyl group, and the cycloalkyl group of R₂₀₄ and R₂₀₅, the substituents that may be included in an aryl group, an alkyl group, and a cycloalkyl group of R₂₀₁ to R₂₀₃ in the aforementioned Formula (ZI) can be exemplified, and examples thereof include an alkyl group (for example, having 1 to 15 carbon atoms), a cycloalkyl group (for example, having 3 to 15 carbon atoms), an aryl group (for example, having 6 to 15 carbon atoms), an alkoxy group (for example, having 1 to 15 carbon atoms), a halogen atom, a hydroxyl group, and a phenylthio group.

Specific examples of the cation represented by Formula (ZII) are provided.

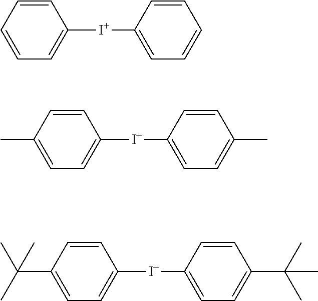

Preferable examples of the cation represented by Formula (ZI) include a cation represented by Formula (7).

(RₘA⁺R_{N})ₙ (7)

In the formula, A represents a sulfur atom.

m represents 1 or 2, and n represents 1 or 2. Here, m+n is 3.

R represents an aryl group.

R_{N} represents an aryl group substituted with a proton acceptor functional group.

The proton acceptor functional group is a group that can electrostatically interact with a proton or a functional group having an electron and means, for example, a functional group having a macrocyclic structure such as cyclic polyether or a functional group having a nitrogen atom having an unshared electron pair that does not contribute to π conjugation. The nitrogen atom having an unshared electron pair that does not contribute to π conjugation is, for example, a nitrogen atom having a partial structure represented by the following formula.

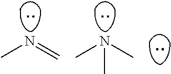

Unshared electron pair

Examples of preferable partial structures of the proton acceptor functional group include a crown ether structure, an azacrown ether structure, a primary to tertiary amine structure, a pyridine structure, an imidazole structure, and a pyrazine structure.

A compound (PA) having a proton acceptor functional group generates a compound which is decomposed by irradiation with actinic rays or radiation and in which proton acceptor properties decrease or disappear or proton acceptor properties change to acidity. Here, the decrease or disappearance of the proton acceptor properties or the change from proton acceptor properties to acidity is a change in the proton acceptor properties due to the addition of a proton to the proton acceptor functional group, and specifically means that, in a case where a proton adduct is generated from the compound (PA) having a proton acceptor functional group and a proton, an equilibrium constant in the chemical equilibrium thereof decreases.

The proton acceptor properties can be checked by performing pH measurement.

Specific examples of the cation represented by Formula (7) are provided. In the following formula, Et represents an ethyl group.

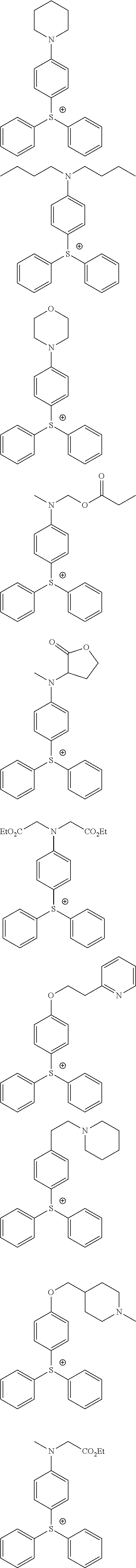

### Compound Having Nonionic Compound Structure

The compound which generates an acid represented by Formula (I) by irradiation with an actinic ray or radiation may have a nonionic compound structure, and examples thereof include a compound represented by Formula (ZV) or (ZVI).

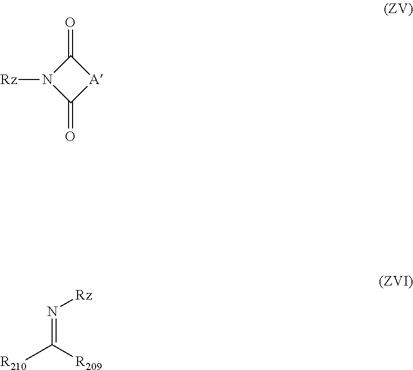

In Formulae (ZV) and (ZVI),

R₂₀₉ and R₂₁₀ each independently represent an alkyl group, a cycloalkyl group, a cyano group, or an aryl group. The aryl group, the alkyl group, and the cycloalkyl group of R₂₀₉ and R₂₁₀ are the same as each group described as the aryl group, the alkyl group, and the cycloalkyl group of R₂₀₁ to R₂₀₃ in the Formula (ZI). An aryl group, an alkyl group, and a cycloalkyl group of R₂₀₉ and R₂₁₀ each may have a substituent. Examples of the substituent include the same substituents as the substituent that may be included in an aryl group, an alkyl group, and a cycloalkyl group of R₂₀₁ to R₂₀₃ in the Formula (ZI).

J represents an alkylene group, an alkenylene group, or an arylene group.

The alkylene group as A′ may have a substituent, and preferably has 1 to 8 carbon atoms, and examples thereof include a methylene group, an ethylene group, a propylene group, a butylene group, a hexylene group, and an octylene group.

The alkenylene group as A′ may have a substituent, and preferably has 2 to 6 carbon atoms, and examples thereof include an ethenylene group, a propenylene group, and a butenylene group.

The arylene group as A′ may have a substituent, and preferably has 6 to 15 carbon atoms, and examples thereof include a phenylene group, a tolylene group, and a naphthylene group.

Examples of the substituent that may be included in A′ include a substituent having active hydrogen such as a cycloalkyl group, an aryl group, an amino group, an amide group, a ureido group, an urethane group, a hydroxyl group, and a carboxy group, and also include a halogen atom (such as a fluorine atom, a chlorine atom, a bromine atom, and an iodine atom), an alkoxy group (such as a methoxy group, an ethoxy group, a propoxy group, and a butoxy group), a thioether group, an acyl group (such as an acetyl group, a propanoyl group, and a benzoyl group), an acyloxy group (such as an acetoxy group, a propanoyloxy group, and a benzoyloxy group), an alkoxycarbonyl group (such as a methoxycarbonyl group, an ethoxycarbonyl group, and a propoxycarbonyl group), a cyano group, and a nitro group. Examples of the arylene group may further include an alkyl group (a methyl group, an ethyl group, a propyl group, and a butyl group).

Rz represents a structure obtained by dissociating H of an acid represented by Formula (I) and is represented by Formula (I-S).

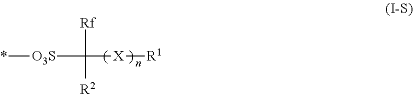

In Formula (I-S), R¹, R², Rf, X, and n have the same meaning as R¹, R², Rf, X, and n in Formula (I). * represents a bonding portion to a compound residue represented by Formula (ZV) or (ZVI).

Specific examples of the compound residue represented by Formula (ZV) or (ZVI) are provided below The symbol * in the specific examples represents a bonding portion to * in Formula (I-S). Me represents a methyl group.

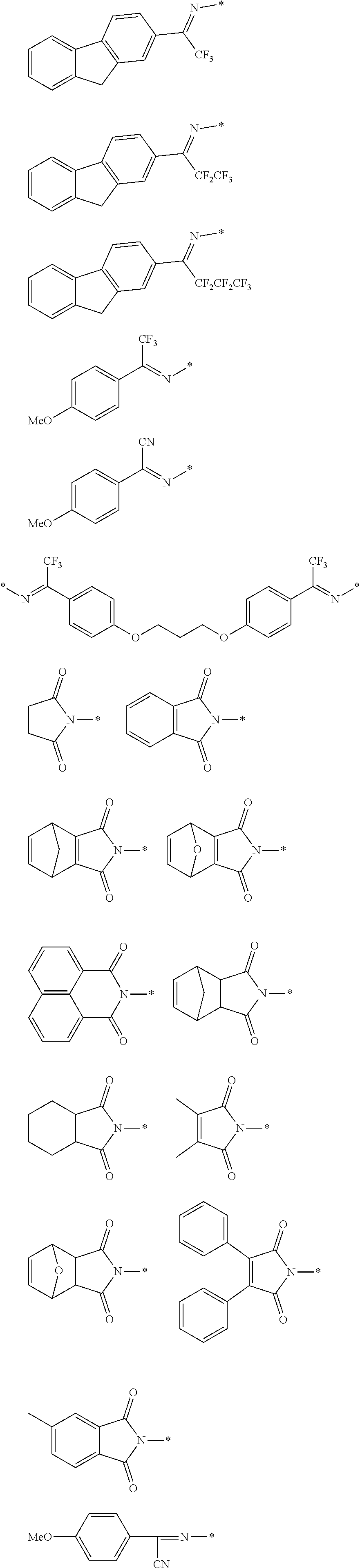

Specific examples of the compound that generates an acid represented by Formula (I) by irradiation with an actinic ray or radiation are provided below.

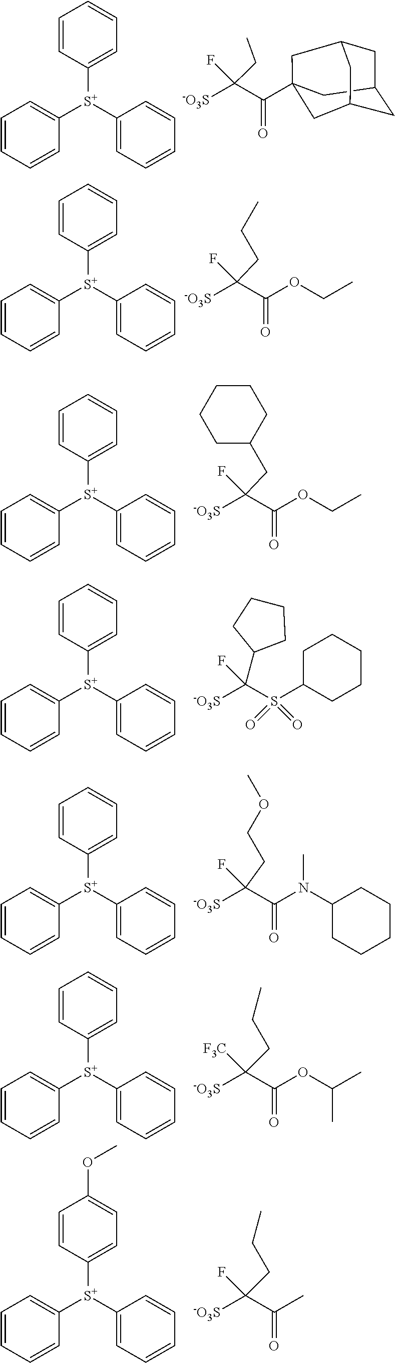

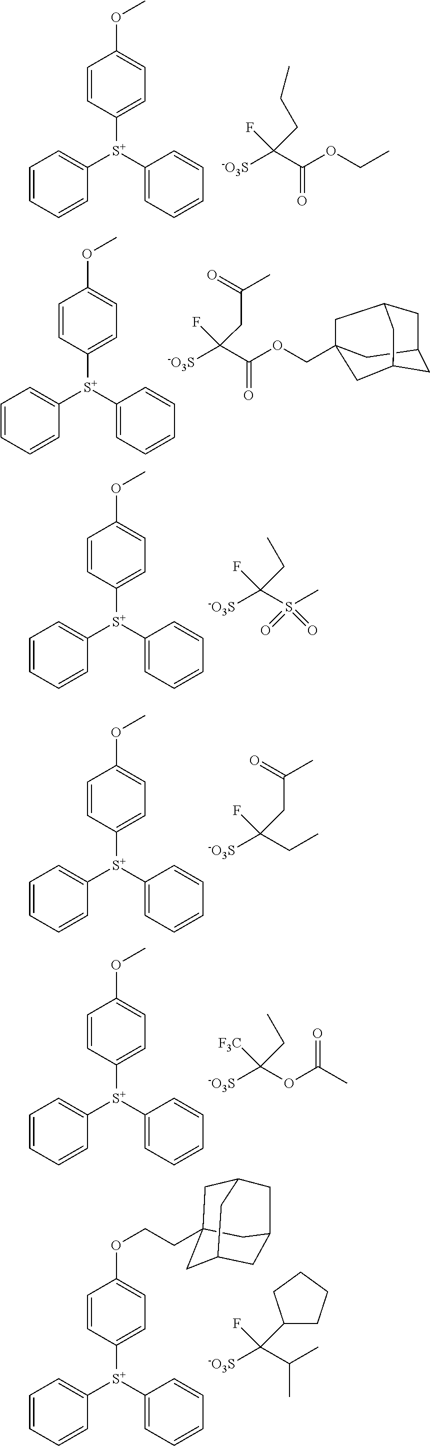

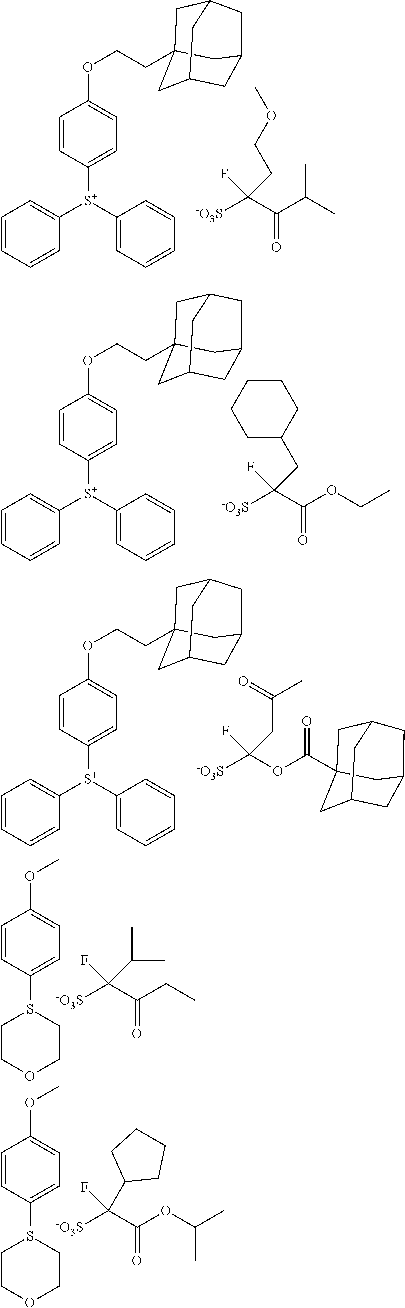

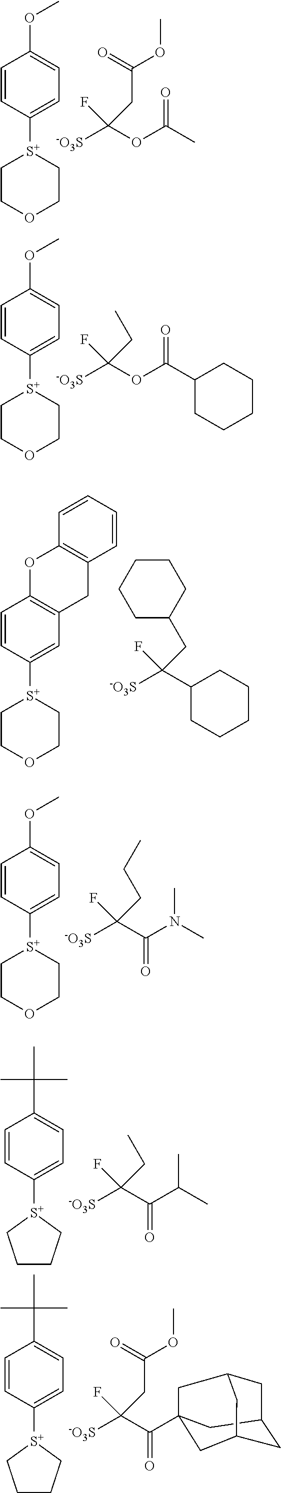

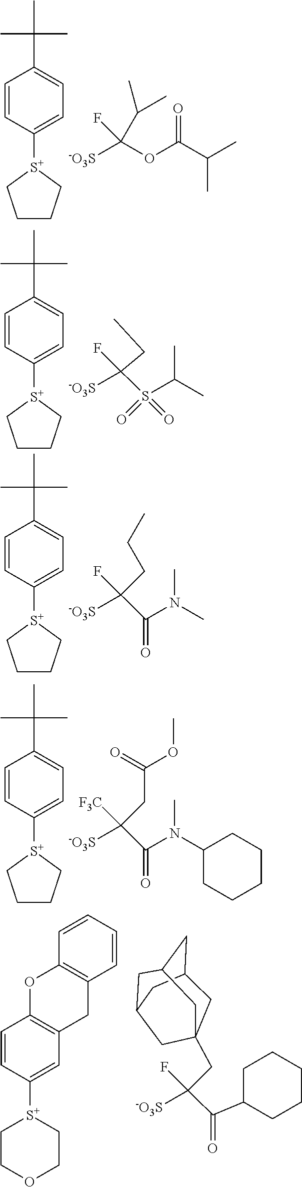

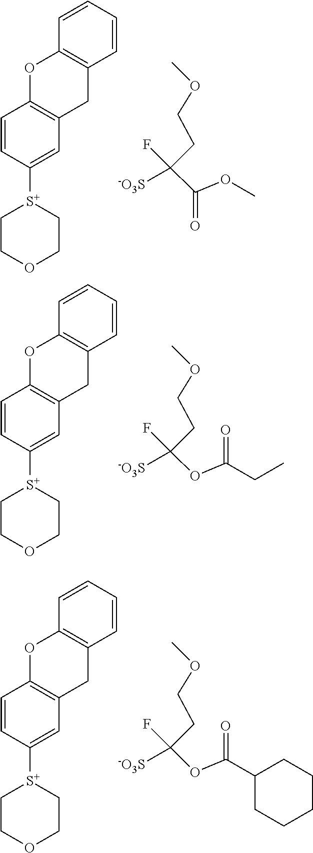

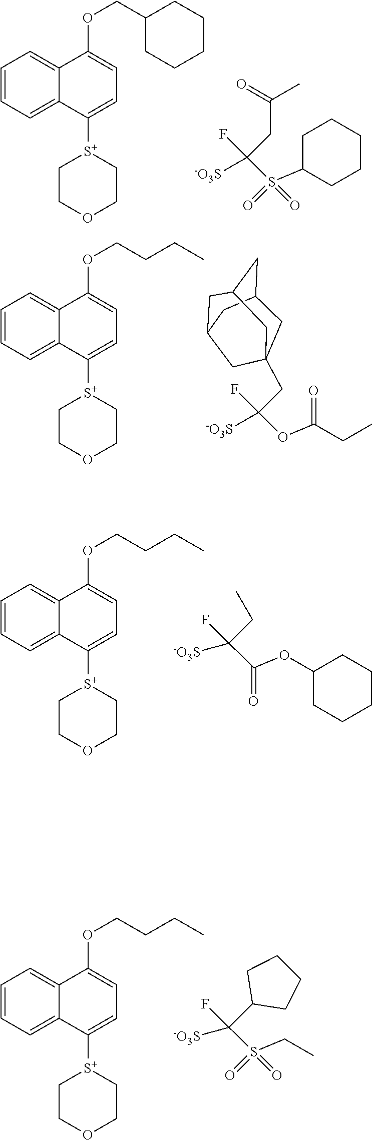

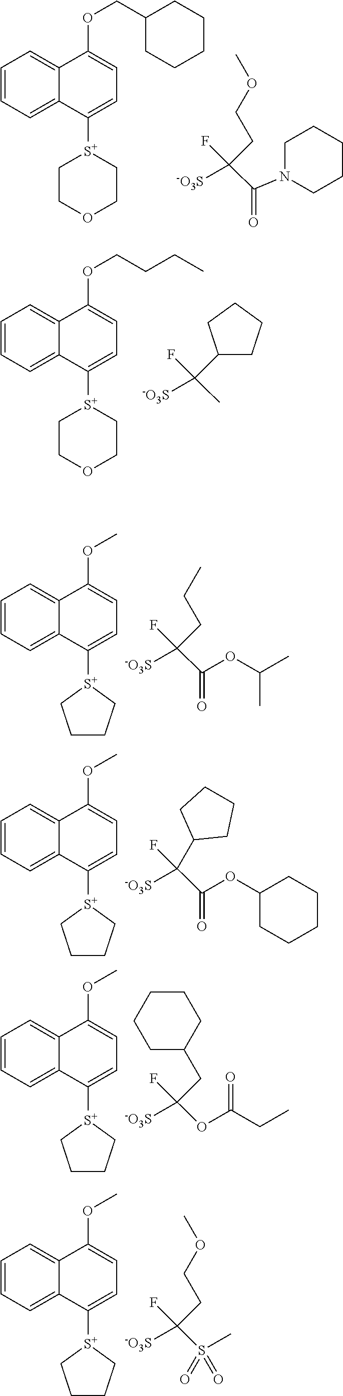

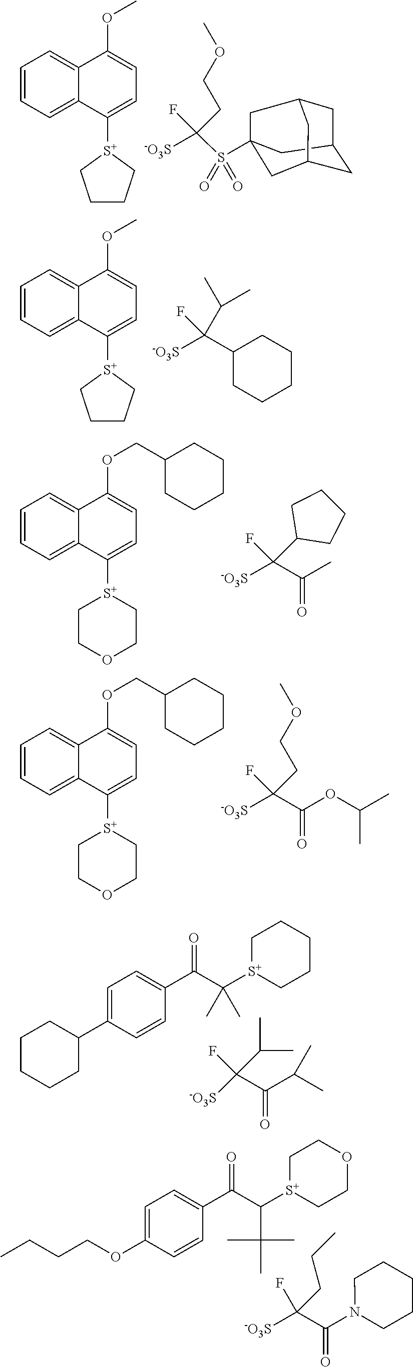

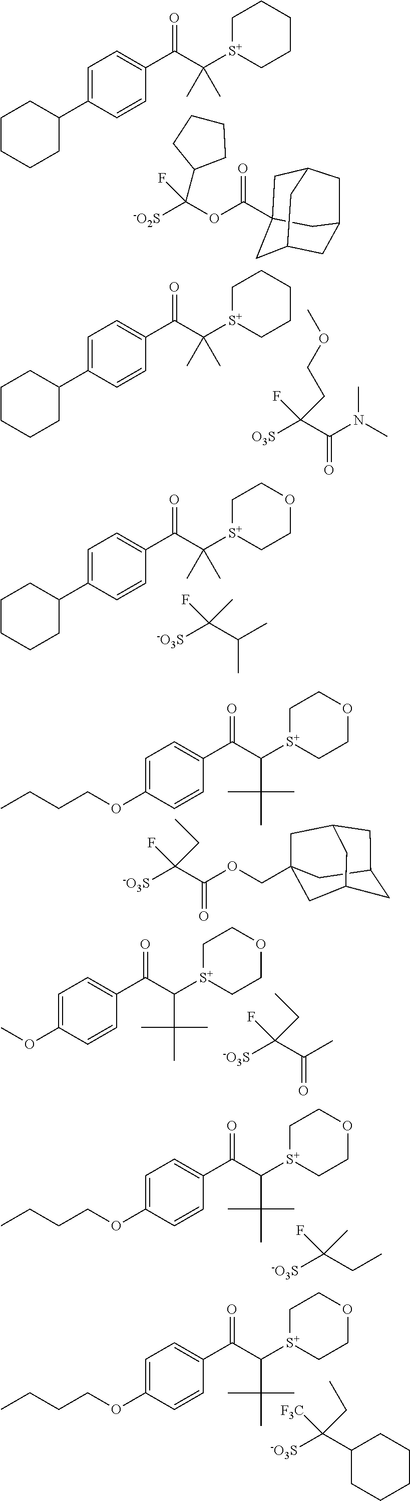

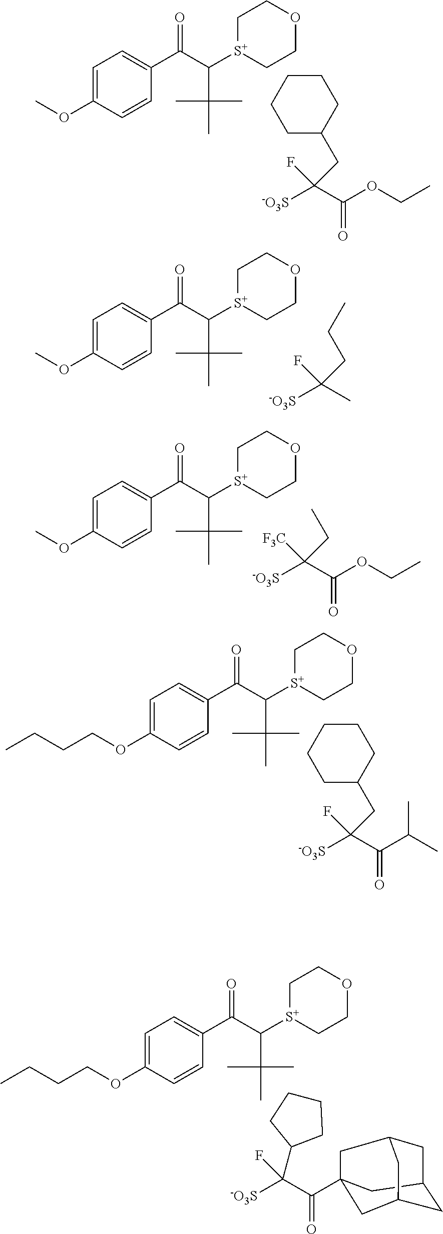

The method of synthesizing a compound that generates an acid represented by Formula (I) due to the irradiation of an actinic ray or radiation can be synthesized by a well-known synthesis method.

In the actinic ray-sensitive or radiation-sensitive resin composition according to the embodiment of the present invention, the compound that generates the acid represented by Formula (I) due to the irradiation of an actinic ray or radiation may be used singly or two or more kinds thereof may be used in combination. Well-known acid generators other than the compound that generates an acid represented by Formula (I) by irradiation with an actinic ray or radiation may be used in combination.

In the case where a well-known acid generator is used, for example, a photoinitiator for photocationic polymerization, a photoinitiator for photoradical polymerization, a light-decoloring agent for coloring agents, a photochromic agent, or well-known compounds that generate an acid due to the irradiation with an actinic ray or radiation used in a micro resist or the like can be appropriately selected to be used.

The content of the acid generator in the actinic ray-sensitive or radiation-sensitive resin composition according to the embodiment of the present invention is preferably 0.1 to 20 mass %, more preferably 0.5 to 20 mass %, and even more preferably 5 to 20 mass % with respect to the total solid content of the actinic ray-sensitive or radiation-sensitive resin composition.

By causing the content of the acid generator to be in this range, the exposure margin in a case where the resist pattern is formed is improved.

In a case where the actinic ray-sensitive or radiation-sensitive resin composition according to the embodiment of the present invention contains two or more kinds of acid generators, a total content of the acid generator is preferably in the above range.

In the acid generator, the compound that generates the acid represented by Formula (I) due to the irradiation of an actinic ray or radiation and another acid generator may be used together, but the content of the compound that generates the acid represented by Formula (I) due to the irradiation of an actinic ray or radiation is preferably 50 mass % or more, more preferably 85 mass % or more, even more preferably 90 mass % or more, and particularly preferably 95 mass % or more with respect to a total mass of the used acid generator.

### Resin

The actinic ray-sensitive or radiation-sensitive resin composition according to the embodiment of the present invention contains a resin.

As the resin, it is possible to use a well-known resin that can form a resist pattern but a resin (hereinafter, referred to as a “resin (A)”) in which polarity changes due to an action of an acid is preferable.

Among these, the resin (A) is more preferably a resin (A1) that is decomposed due to an action of an acid to increase polarity. That is, the resin (A) is a resin in which solubility in an alkali developer increases due to an action of an acid, or solubility in a developer with an organic solvent as a main component due to an action of an acid decreases, and specifically, a resin having a group (hereinafter also referred to as an “acid-decomposable group”) that generates an alkali-soluble group by being decomposed due to an action of an acid on at least one of a main chain or a side chain.

Examples of the alkali-soluble group include a carboxy group, a fluorinated alcohol group (preferably a hexafluoroisopropanol group), and a sulfonic acid group.

Hereinafter, the resin (A) is described in detail.

### Repeating Unit Having Acid-Decomposable Group

The resin (A) preferably has a repeating unit having an acid-decomposable group as described above. A repeating unit that has acid-decomposable group is preferably a repeating unit represented by Formula (AI).

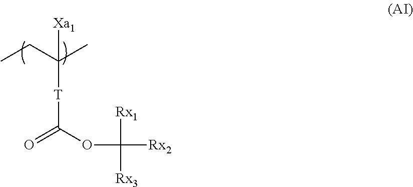

In Formula (AI),

Xa₁ represents a hydrogen atom or an alkyl group that may have a substituent.

T represents a single bond or a divalent linking group.

Rx₁ to Rx₃ each independently represent a (linear or branched) alkyl group or a (monocyclic or polycyclic) cycloalkyl group.

Two of Rx₁ to Rx₃ are bonded to form a (monocyclic or polycyclic) cycloalkyl group.

Examples of the alkyl group that is represented by Xa₁ and may have a substituent include a methyl group and a group represented by —CH₂—R₁₁. R₁₁ represents a halogen atom (such as a fluorine atom), a hydroxyl group, or a monovalent organic group.

According to an aspect, Xa₁ is preferably a hydrogen atom, a methyl group, a trifluoromethyl group, or a hydroxymethyl group

Examples of the divalent linking group of T include an alkylene group, a —COO—Rt- group, and an —O—Rt- group. In the formula, Rt represents an alkylene group or a cycloalkylene group.

preferably a single bond or a —COO—Rt- group. Rt is preferably an alkylene group having 1 to 5 carbon atoms and more preferably a —CH₂— group, a —(CH₂)₂— group, and a —(CH₂)₃— group.

The alkyl group of Rx₁ to Rx₃ preferably has 1 to 4 carbon atoms.

The cycloalkyl group of Rx₁ to Rx₃ is preferably a monocyclic cycloalkyl group such as a cyclopentyl group or a cyclohexyl group, a polycyclic cycloalkyl group such as a norbornyl group, a tetracyclodecanyl group, a tetracyclododecanyl group, or an adamantyl group.

The cycloalkyl group formed by bonding two of Rx₁ to Rx₃ is preferably a monocyclic cycloalkyl group such as a cyclopentyl group and a cyclohexyl group or a polycyclic cycloalkyl group such as a norbornyl group, a tetracyclodecanyl group, a tetracyclododecanyl group, or an adamantyl group. The monocyclic cycloalkyl group having 5 to 6 carbon atoms is more preferable.

With respect to the cycloalkyl group formed by bonding two of Rx₁ to Rx₃, for example, one of the methylene groups constituting the ring may be substituted with a hetero atom such as an oxygen atom or a group having a hetero atom such as a carbonyl group.

It is preferable that the repeating unit represented by Formula (AI), for example, is an aspect in which Rx₁ is a methyl group or an ethyl group, and in which and Rx₂ and Rx₃ are bonded to each other to form the above cycloalkyl group.

Each of the above groups may have a substituent, examples of the substituent include an alkyl group (having 1 to 4 carbon atoms), a halogen atom, a hydroxyl group, an alkoxy group (having 1 to 4 carbon atoms), a carboxy group, and an alkoxycarbonyl group (having 2 to 6 carbon atoms), and a group having 8 or less carbon atoms is preferable.

The content of the sum of the repeating units having acid-decomposable groups is preferably 20 to 90 mol %, more preferably 25 to 85 mol %, and even more preferably 30 to 80 mol % with respect to the all repeating units in the resin (A).

Specific examples of the repeating unit having an acid-decomposable group are provided below, but the present invention is not limited thereto.

In the specific examples, Rx and Xa₁ each independently represent a hydrogen atom, CH₃, CF₃, or CH₂OH. Rxa and Rxb each represent an alkyl group having 1 to 4 carbon atoms. Z represents a substituent including a polar group, and in a case where there are a plurality of Z's, Z's each independently represent a substituent including a polar group. p represents 0 or a positive integer. Examples of the substituent including a polar group represented by Z include a linear or branched alkyl group having a hydroxyl group, a cyano group, an amino group, an alkylamido group, or a sulfonamide group, or a cycloalkyl group, and the substituent is preferably an alkyl group having a hydroxyl group. The branched alkyl group is more preferably an isopropyl group.

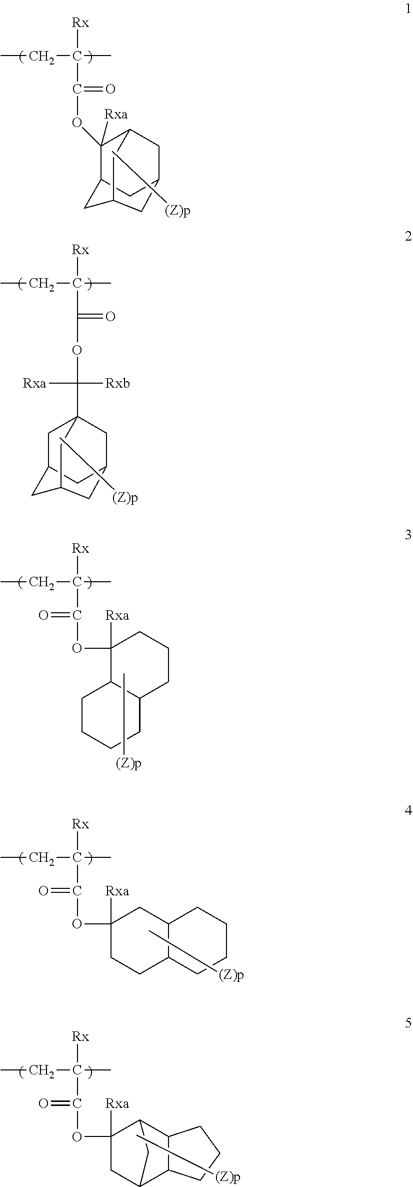

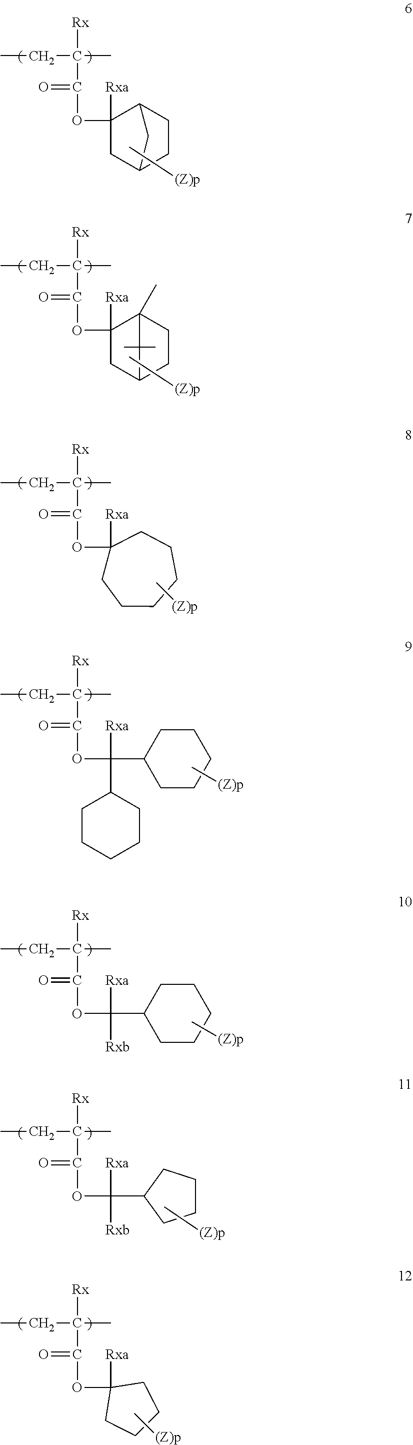

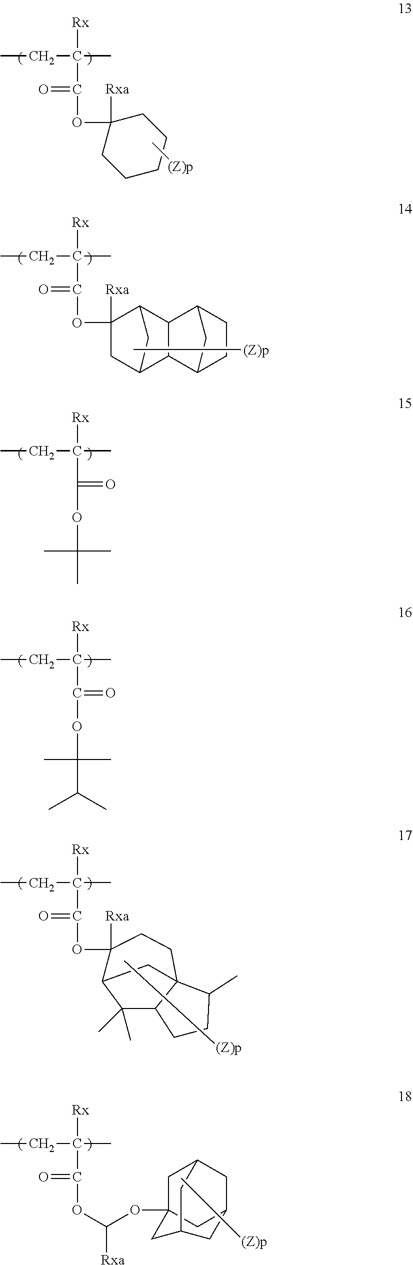

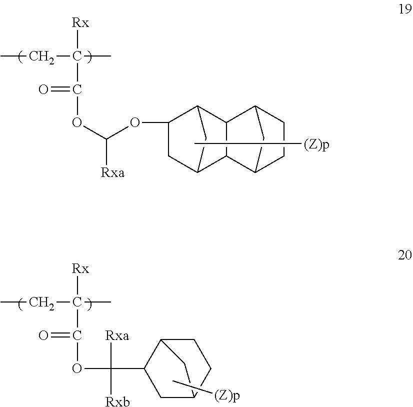

### Repeating Unit Having Lactone Structure or Sultone Structure

The resin (A) preferably contains a repeating unit having a lactone structure or a sultone (cyclic sulfonic acid ester) structure.

The repeating unit having a lactone structure or a sultone structure preferably has a lactone structure or a sultone structure in a side chain and more preferably, for example, a repeating unit derived from a (meth)acrylic acid derivative monomer.

The repeating unit having a lactone structure or a sultone structure may be used singly or two or more kinds thereof may be used in combination, but it is preferable to use the repeating unit singly.

The content of the repeating unit having a lactone structure or a sultone structure with respect to all repeating units of the resin (A) is, for example, 3 to 80 mol %, and preferably 3 to 60 mol %.

The lactone structure is preferably a lactone structure of a 5-membered to 7-membered ring and more preferably a structure in which another ring structure is fused in a form of forming a bicyclo structure or a spiro structure in a lactone structure of a 5-membered to 7-membered ring.

It is preferable that the lactone structure has a repeating unit having a lactone structure represented by any one of Formulae (LC1-1) to (LC1-17). The lactone structure is preferably the lactone structure represented by Formula (LC1-1), (LC1-4), (LC1-5), or (LC1-8) and more preferably the lactone structure represented by Formula (LC1-4).

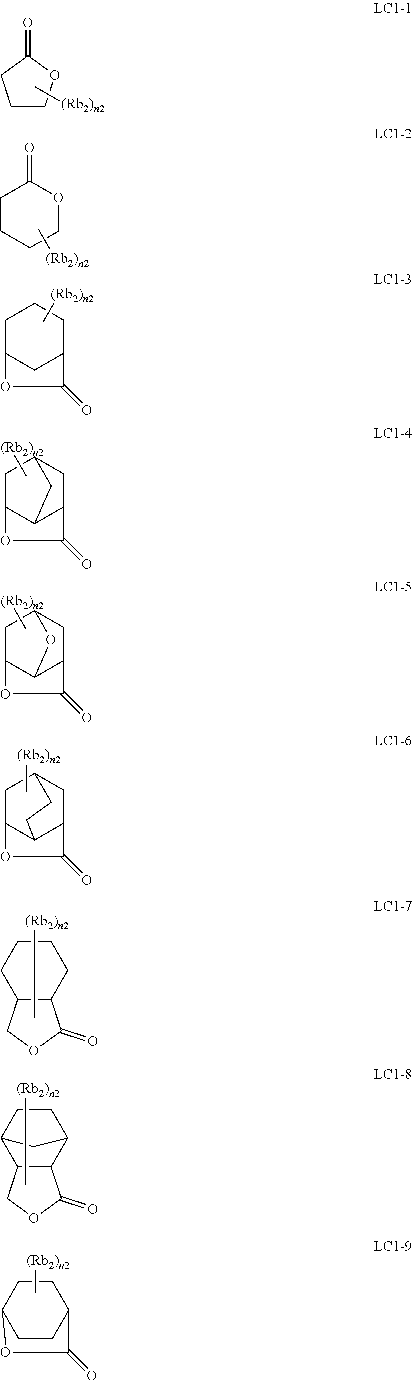

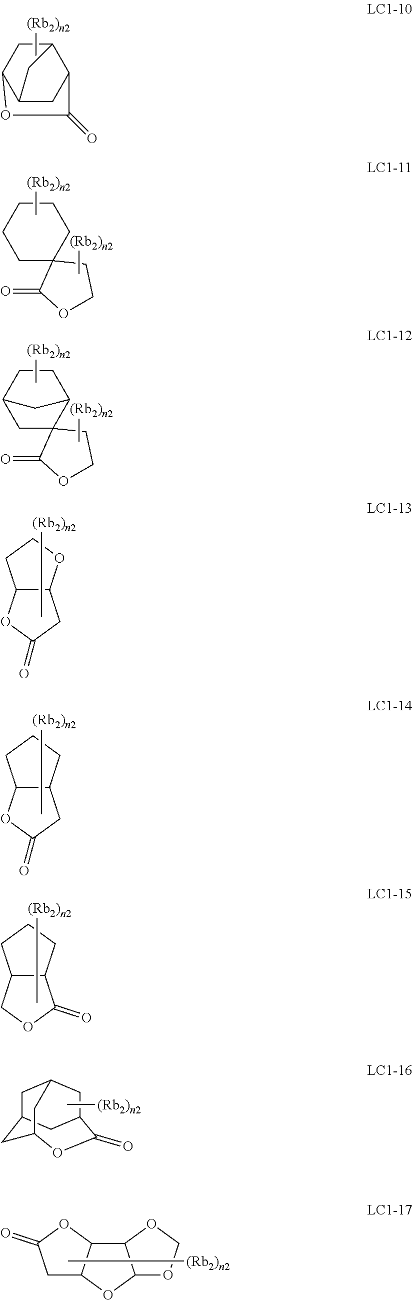

A lactone structure portion may have a substituent (Rb₂). Preferable examples of the substituent (Rb₂) include an alkyl group having 1 to 8 carbon atoms, a cycloalkyl group having 4 to 7 carbon atoms, an alkoxy group having 1 to 8 carbon atoms, an alkoxycarbonyl group having 2 to 8 carbon atoms, a carboxy group, a halogen atom, a hydroxyl group, a cyano group, and an acid-decomposable group. n₂ represents an integer of 0 to 4. In a case where n₂ is 2 or more, the plurality of substituents (Rb₂) which are present may be identical to or different from each other, and the plurality of substituent (Rb₂) which are present may be bonded to each other to form a ring.

The sultone structure is preferably a sultone structure of a 5-membered to 7-membered ring and more preferably a structure in which another ring structure is fused in a form of forming a bicyclo structure or a spiro structure in a sultone structure of a 5-membered to 7-membered ring.

It is preferable that the sultone structure has a repeating unit having a sultone structure represented by any one of Formulae (SL1-1) and (SL1-2). A sultone structure may be directly bonded to a main chain.

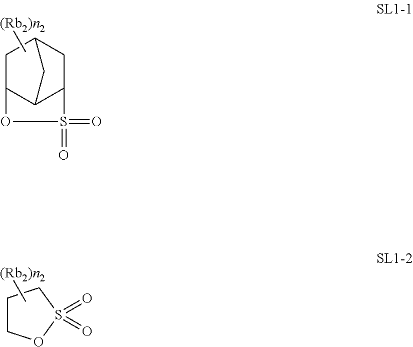

A sultone structure portion may have a substituent (Rb₂). In the above formula, the substituent (Rb₂) and n₂ have the same meaning as the substituent Rb₂) and n₂ of the lactone structure portion.

The repeating unit having a lactone structure or a sultone structure is preferably a repeating unit represented by Formula (III).

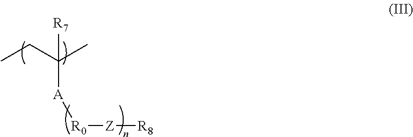

In Formula (III),

A represents an ester bond (a group represented by —COO—) or an amide bond (a group represented by —CONH—).

In a case where there are a plurality of R₀'s, R₀'s each independently represent an alkylene group, a cycloalkylene group, or a combination thereof.

In a case where there are a plurality of Z's, Z's each independently represent a single bond, an ether bond, an ester bond, an amide bond, a urethane bond

(a group represented by

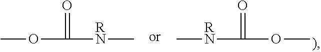

or a urea bond

(a group represented by

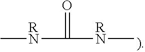

Here, R's each independently represent a hydrogen atom, an alkyl group, a cycloalkyl group, and an aryl group.

R₈ represents a monovalent organic group having a lactone structure or a sultone structure.

n is the number of repetitions of the structure represented by —R₀—Z—, and represents an integer of 0 to 2.

R₇ represents a hydrogen atom, a halogen atom, or an alkyl group.

The alkylene group and the cycloalkylene group of R₀ may have a substituent.

Z is preferably an ether bond or an ester bond and more preferably an ester bond.

The alkyl group of R₇ is preferably an alkyl group having 1 to 4 carbon atoms, more preferably a methyl group or an ethyl group, and even more preferably a methyl group. The alkylene group and the cycloalkylene group of R₀ and the alkyl group of R₇ each may be substituted. R₇ is preferably a hydrogen atom, a methyl group, a trifluoromethyl group, or a hydroxymethyl group.

A chain alkylene group in R₀ is preferably a chain alkylene having 1 to 10 carbon atoms, more preferably a chain alkylene having 1 to 5 carbon atoms. A preferable cycloalkylene group is a cycloalkylene group having 3 to 20 carbon atoms. Among these, a chain alkylene group is more preferable, and a methylene group is even more preferable.

The monovalent organic group having a lactone structure or a sultone structure represented by R₈ is not limited, as long as the monovalent organic group has a lactone structure or a sultone structure, and specific examples thereof include a lactone structure represented by Formulae (LC1-1) to (LC1-17), or a sultone structure represented by Formulae (SL1-1) and (SL1-2), and among these, a structure represented by Formula (LC1-4) is preferable. n₂'s in Formulae (LC1-1) to (LC1-17), Formula (SL1-1), and Formula (SL1-2) each are more preferably 2 or less.

R₈ is preferably a monovalent organic group having an unsubstituted lactone structure or an unsubstituted sultone structure or a monovalent organic group having a lactone structure or a sultone structure having a methyl group, a cyano group, an N-alkoxyamide group, or an alkoxycarbonyl group as a substituent and more preferably a monovalent organic group having a lactone structure (cyano lactone) or sultone structure (cyano sultone) having a cyano group as a substituent.

In Formula (III), n is preferably 1 or 2.

### Repeating Unit Having Carbonate Structure

The resin (A) may have a repeating unit having a carbonate structure.

The carbonate structure (cyclic carbonic acid ester structure) is a structure having a ring including a bond represented by —O—C(═O)—O— as the atomic group constituting the ring. A ring including a bond represented by —O—C(═O)—O— as the atomic group constituting the ring is preferably a 5-membered to 7-membered ring and more preferably a 5-membered ring. The ring may be fused with another ring to form a fused ring.

The resin (A) preferably contains a repeating unit represented by Formula (A-1) as a repeating unit having a carbonate structure (cyclic carbonic acid ester structure).

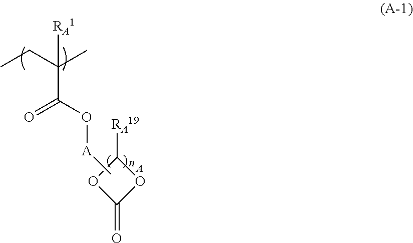

In Formula (A-1), R_{A}¹ represents a hydrogen atom or an alkyl group.

R_{A}¹⁹ each independently represent a hydrogen atom or a chain hydrocarbon group.

A represents a single bond, a divalent or trivalent chain hydrocarbon group, a divalent or trivalent alicyclic hydrocarbon group or a divalent or trivalent aromatic hydrocarbon group, and in a case where A is trivalent, a carbon atom included in A and a carbon atom constituting cyclic carbonic acid ester are bonded to each other so as to form a ring structure.

n_{A} represents an integer of 2 to 4.

In Formula (A-1), R_{A}¹ represents a hydrogen atom or an alkyl group. The alkyl group represented by R_{A}¹ may have a substituent such as a fluorine atom. R_{A}¹ preferably represents a hydrogen atom, a methyl group, or a trifluoromethyl group and more preferably represents a methyl group.

R_{A}¹⁹ each independently represent a hydrogen atom or a chain hydrocarbon group. The chain hydrocarbon group represented by R_{A}¹⁹ is preferably a chain hydrocarbon group having 1 to 5 carbon atoms. Examples of the chain hydrocarbon group having 1 to 5 carbon atoms include a linear alkyl group having 1 to 5 carbon atoms such as a methyl group, an ethyl group, a propyl group, or a butyl group; a branched alkyl group having 3 to 5 carbon atoms such as an isopropyl group, an isobutyl group, or a t-butyl group. The chain hydrocarbon group may have a substituent such as a hydroxyl group.

R_{A}¹⁹ more preferably represents a hydrogen atom.

In Formula (A-1), n_{A} represents an integer of 2 to 4. That is, the cyclic carbonic acid ester has a 5-membered ring structure in a case of n=2 (ethylene group), a 6-membered ring structure in a case of n=3 (propylene group), and a 7-membered ring structure in the case of n=4 (butylene group). For example, the repeating unit (A-1a) described below is an example of a 5-membered ring structure, and repeating unit (A-1j) is an example of a 6-membered ring structure.

n_{A} is preferably 2 or 3 and more preferably 2.

In Formula (A-1), A represents a single bond, a divalent or trivalent chain hydrocarbon group, a divalent or trivalent alicyclic hydrocarbon group, or a divalent or trivalent aromatic hydrocarbon group.

The divalent or trivalent chain hydrocarbon group is preferably a divalent or trivalent chain hydrocarbon group having 1 to 30 carbon atoms.

The divalent or trivalent alicyclic hydrocarbon group is preferably a divalent or trivalent alicyclic hydrocarbon group having 3 to 30 carbon atoms.

The divalent or trivalent aromatic hydrocarbon group is preferably a divalent or trivalent aromatic hydrocarbon group having 6 to 30 carbon atoms.

In a case where A is a single bond, an oxygen atom of (alkyl)acrylic acid (typically (meth)acrylic acid) which constitutes the polymer and in which R_{A}¹ is bonded to an α-position and a carbon atom which constitutes cyclic carbonic acid ester are directly bonded to each other.

A preferably represents a divalent or trivalent chain hydrocarbon group or a divalent or trivalent alicyclic hydrocarbon group, more preferably represents a divalent or trivalent chain hydrocarbon group, and even more preferably represents a linear alkylene group having 1 to 5 carbon atoms.

The monomer can be synthesized by methods well-known in the related art disclosed in Tetrahedron Letters, Vol. 27, No. 32, p. 3741 (1986), Organic Letters, Vol. 4, No. 15, p. 2561 (2002), or the like.

Specific examples (repeating units (A-1a) to(A-1w)) of the repeating unit represented by Formula (A-1) are provided below, but the present invention is not limited to these.

In the following specific examples, R_{A}¹ has the same meaning as R_{A}¹ in Formula (A-1).

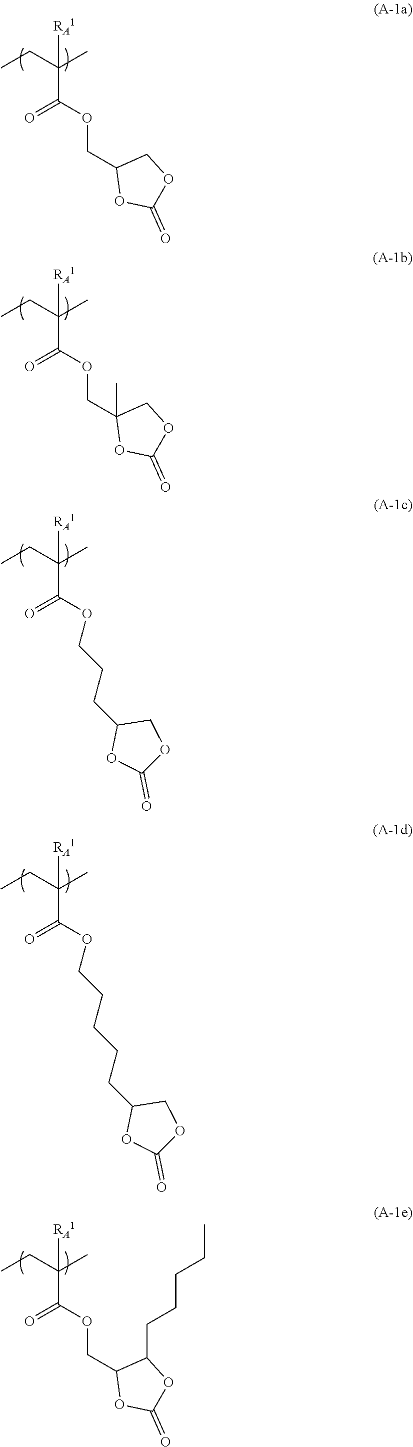

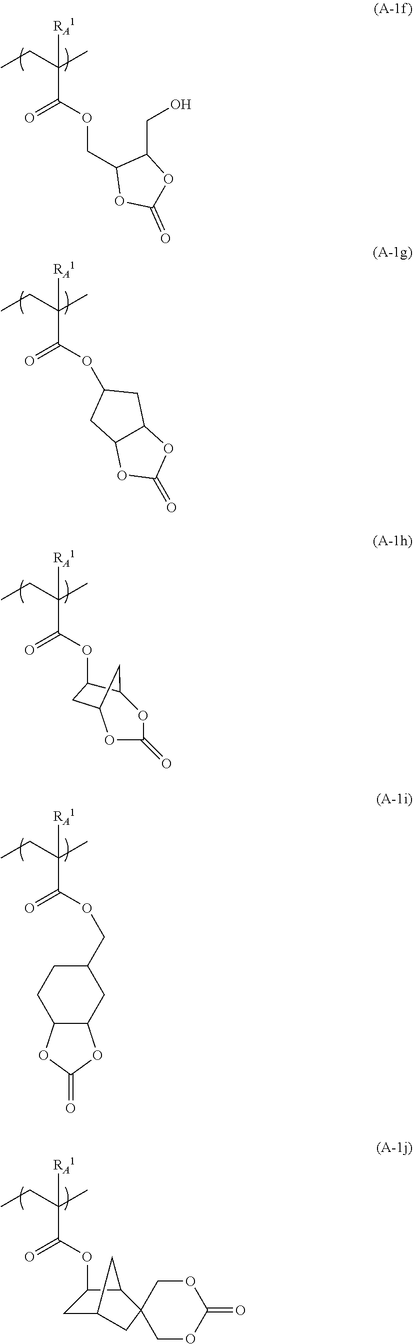

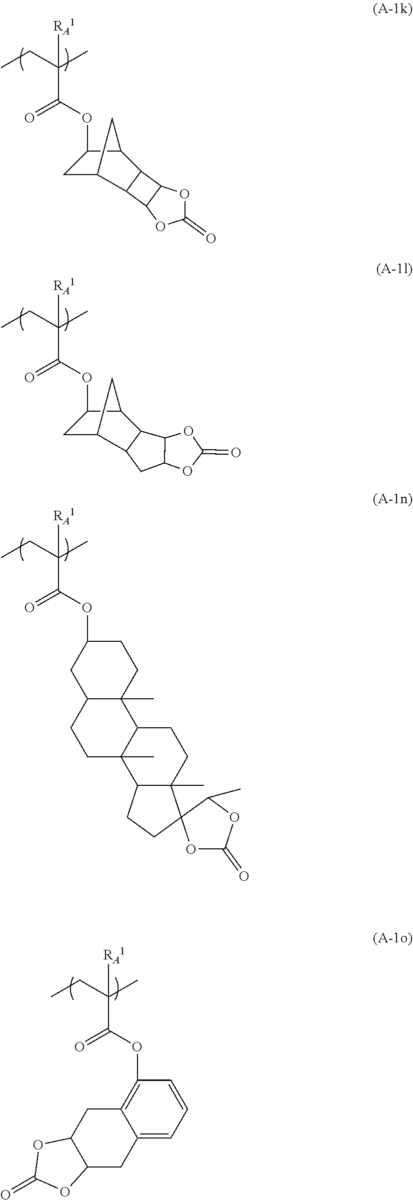

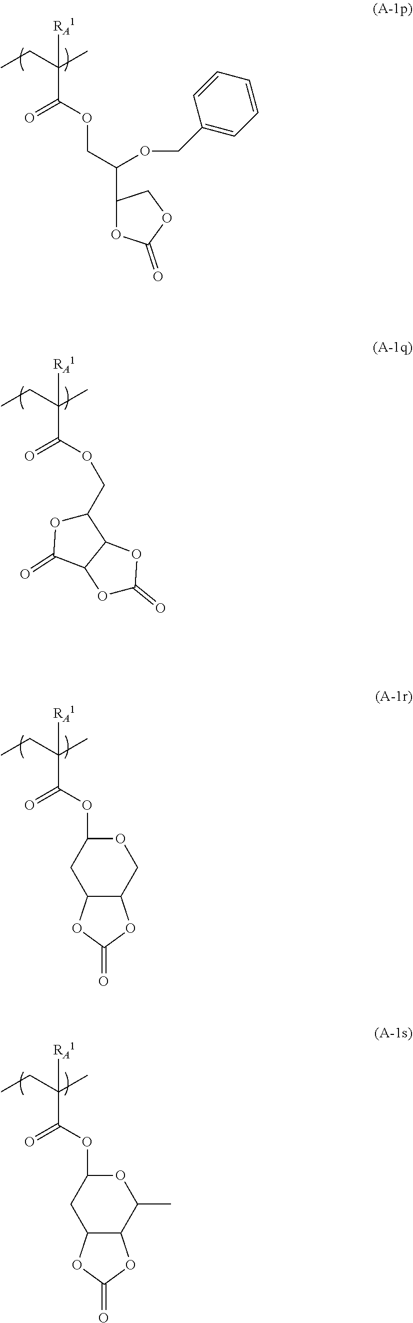

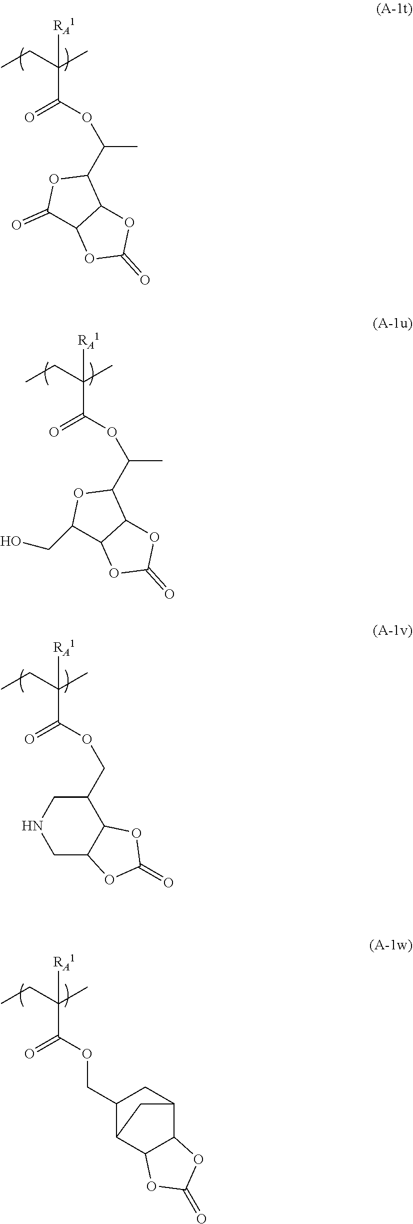

In the resin (A), one kind of repeating units represented by Formula (A-1) may be included singly or two or more kinds thereof may be included.

In the resin (A), the content ratio of the repeating unit having a carbonate structure (cyclic carbonic acid ester structure) (preferably the repeating unit represented by Formula (A-1)) is preferably 3 to 80 mol %, more preferably 3 to 60 mol %, and even more preferably 3 to 30 mol % with respect to all repeating units of the resin (A).

### Repeating Unit in Which Lactone Structure is Directly Connected to Main Chain

The resin (A) may have a repeating unit in which a lactone structure is directly connected to a main chain.

The repeating unit in which a lactone structure is directly connected to a main chain is preferably a repeating unit represented by Formula (q1).

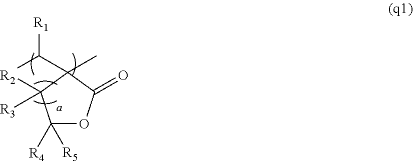

In Formula (q1), R₁ represents a hydrogen atom or an organic group having 1 to 20 carbon atoms. R₂ to R₅ each independently represent a hydrogen atom, a fluorine atom, a hydroxyl group, or an organic group having 1 to 20 carbon atoms. a represents an integer of 1 to 6. Here, R₂ and R₃, and R₄ and R₅ may be bonded to each other to form a ring structure having 3 to 10 ring members together with a carbon atom to which these are bonded.

In Formula (q1), R₁ represents a hydrogen atom or an organic group having 1 to 20 carbon atoms.

Examples of the organic group having 1 to 20 carbon atoms represented by R₁ in Formula (q1) include a chain hydrocarbon group having 1 to 20 carbon atoms, an alicyclic hydrocarbon group having 3 to 20 carbon atoms, an aromatic hydrocarbon group having 6 to 20 carbon atoms, a heterocyclic group having 3 to 10 ring members, an epoxy group, a cyano group, a carboxy group, or a group represented by —R′-Q-R″. Here, R′ is a single bond or a hydrocarbon group having 1 to 20 carbon atoms. R″ is a hydrocarbon group having 1 to 20 carbon atoms which may be substituted or a heterocyclic group having 3 to 10 ring members. Q is —O—, —CO—, —NH—, —SO₂—, —SO—, or a group obtained by combining these. A portion or all of the hydrogen atoms that are included in the chain hydrocarbon group, the alicyclic hydrocarbon group, and the aromatic hydrocarbon group may be substituted with, for example, a halogen atom such as a fluorine atom or a substituent such as a cyano group, a carboxy group, a hydroxyl group, a thiol group, or a trialkylsilyl group.

In Formula (q1), R₁ is preferably a hydrogen atom in view of the copolymerizability of a monomer providing a repeating unit in which a lactone structure is directly connected to a main chain.

In Formula (q1), R₂ to R₅ each independently represent a hydrogen atom, a fluorine atom, a hydroxyl group, or an organic group having 1 to 20 carbon atoms.

Specific examples and suitable aspects of the organic group having 1 to 20 carbon atoms represented by R₂ to R₅ in Formula (q1) are the same as the organic group having 1 to 20 carbon atoms represented by R₁ in Formula (q1).

In Formula (q1), R₂ and R₃, and R₄ and R₅ may be bonded to each other to form a ring structure having 3 to 10 ring members together with carbon atoms to which these are bonded.

Examples of the ring structure having 3 to 10 ring members which is formed by bonding R₂ and R₃, and R₄ and R₅ to each other, together with carbon atoms to which these are bonded include an alicyclic structure having an alicyclic ring such as include cyclopropane, cyclopentane, cyclohexane, norbornane, or adamantane; and a heterocyclic structure having a ring including a hetero atom.

Examples of the heterocyclic structure having a ring including a hetero atom include a heterocyclic structure having a cyclic ether, a lactone ring, or a sultone ring, and other specific examples thereof include a heterocyclic structure having an oxygen atom-containing ring such as tetrahydrofuran, tetrahydropyran, γ-butyrolactone, δ-valerolactone, oxolane, and dioxane; and a heterocyclic structure having a sulfur atom-containing ring such as tetrahydrothiophene, tetrahydrothiopyran, tetrahydrothiophene-1,1-dioxide, tetrahydrothiopyran-1,1-dioxide, cyclopentanethione, and cyclohexanethione; and a heterocyclic structure having a nitrogen atom-containing ring such as piperidine.

Among these, an alicyclic structure having cyclopentane, cyclohexane, or adamantane, and a heterocyclic structure having cyclic ether, a lactone ring, or a sultone ring is preferable.

Here, the “ring structure” in the ring structure having 3 to 10 ring members which may be formed by bonding R₂ and R₃, and R₄ and R₅ to each other together with carbon atoms to which these are bonded refers to a structure containing a ring, and may be formed only from a ring or may be formed from a ring and another group such as a substituent. The bonding in a case where R₂ and R₃, and R₄ and R₅ are bonded to each other is not limited to a bonding by the chemical reaction.

In Formula (q1), a represents an integer of 1 to 6. a is preferably an integer of 1 to 3, more preferably 1 or 2, and even more preferably 1.

In Formula (q1), in a case where a is 2 or more, the plurality of R₂'s and R₃'s may be identical to or different from each other.

R₂ and R₃ each are preferably a hydrogen atom or a chain hydrocarbon group having 1 to 20 carbon atoms and more preferably a hydrogen atom.

R₄ and R₅ each are preferably a hydrogen atom, a chain hydrocarbon group having 1 to 20 carbon atoms, a heterocyclic group having 3 to 10 ring members, or a group that forms a ring structure having 3 to 10 ring members by bonding these to each other together with carbon atoms to which these are bonded.

Examples of the repeating unit represented by Formula (q1) include repeating units represented by the following formula, but the present invention is not limited to these. R₁ in the following formula has the same meaning as R₁ in Formula (q1).

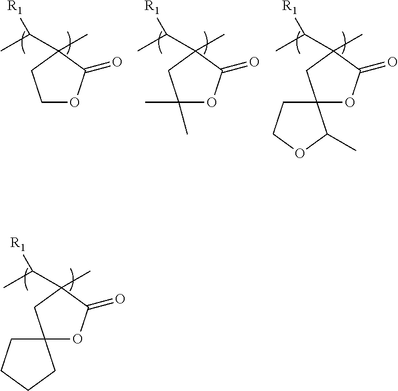

A repeating unit obtained by directly connecting the lactone structure represented by Formula (q1) to a main chain may be used singly or two or more kinds thereof may be used in combination.

With respect to all repeating units of the resin (A), the content of the repeating unit obtained by directly connecting a lactone structure represented by Formula (q1) to a main chain is not particularly limited, but is preferably 5 to 60 mol %, more preferably 5 to 50 mol %, and even more preferably 10 to 40 mol %.

### Other Repeating Units

The resin (A) may include another repeating unit.

For example, the resin (A) may include a repeating unit having a hydroxyl group or a cyano group. Examples of the repeating unit include repeating units disclosed in paragraphs <0081> to <0084> of JP2014-098921A.

The resin (A) may have a repeating unit having an alkali-soluble group. Examples of the alkali-soluble group include a carboxy group, a sulfonamide group, a sulfonylimide group, a bissulfonylimide group, and aliphatic alcohol (for example, hexafluoroisopropanol group) of which the α-position is substituted with an electron withdrawing group. Examples of the repeating unit having an alkali-soluble group include repeating units disclosed in paragraphs <0085> and <0086> of JP2014-098921A.

The resin (A) may further have a repeating unit having an alicyclic hydrocarbon structure which does not have a polar group (for example, alkali-soluble group, a hydroxyl group, and a cyano group) and not exhibiting acid decomposability. Examples of the repeating unit include repeating units disclosed in paragraphs <0114> to <0123> of JP2014-106299A.

For example, the resin (A) may include repeating units disclosed in paragraphs <0045> to <0065> of JP2009-258586A.

The resin (A) used in the composition according to the embodiment of the present invention may include various repeating units in addition to the repeating unit. Examples of the repeating units include repeating units corresponding to the following monomers, but the repeating units are not limited thereto.

Examples of the monomer include a compound having one addition polymerizable unsaturated bond selected from acrylic acid esters, methacrylic acid esters, acrylamides, methacrylamides, allyl compounds, vinyl ethers, and vinyl esters.

In addition, in a case of an addition polymerizable unsaturated compound copolymerizable with the monomer corresponding to the above various repeating structural units, the compound may be copolymerized.

With respect to the resin (A) used in the composition according to the embodiment of the present invention, the content molar ratio of each of the repeating structural units is appropriately set.

In a case where the composition according to the embodiment of the present invention is for ArF exposure, in view of transparency to ArF light, it is preferable that the resin (A) used in the composition according to the embodiment of the present invention has substantially no aromatic group. Specifically, during the entire repeating unit of the resin (A), the content of the repeating unit having an aromatic group is preferably 5 mol % or less, more preferably 3 mol % or less, and ideally 0 mol % with respect to the all repeating units, that is, it is more preferable to not have a repeating unit having an aromatic group. The resin (A) preferably has a monocyclic or polycyclic alicyclic hydrocarbon structure.

The weight-average molecular weight (Mw) of the resin (A) is preferably 1,000 to 200,000 and more preferably 2,000 to 20,000. In a case where the weight-average molecular weight is caused to be 1,000 to 200,000, it is possible to prevent deterioration of heat resistance and dry etching resistance and it is possible to prevent deterioration of developability and deterioration of lam formability due to increase in viscosity.

A dispersion degree (molecular weight distribution) which is a ratio (Mw/Mn) of a weight-average molecular weight (Mw) and a number-average molecular weight (Mn) in the resin (A) is generally 1.0 to 3.0, preferably 1.0 to 2.6, more preferably 1.0 to 2.0, and even more preferably in the range of 1.1 to 2.0. As the molecular weight distribution is smaller, a resolution and a resist shape are excellent, a sidewall of a resist pattern is smooth, and roughness properties are excellent.

A content ratio of the resin (preferably the resin (A)) in the total composition is preferably in the range of 30 to 99 mass % and more preferably in the range of 50 to 95 mass % with respect to the total solid content.

The resin (preferably the resin (A)) may be used singly, and two or more kinds thereof may be used in combination. In a case where two or more resins (preferably the resin (A)) are used in combination, it is preferable that the total content is in the above range.

### Basic Compound

The actinic ray-sensitive or radiation-sensitive resin composition according to the embodiment of the present invention may contain a basic compound.

The basic compound is not particularly limited, and well-known basic compounds can be used.

In a case where the actinic ray-sensitive or radiation-sensitive resin composition contains a basic compound, the content of the basic compound is generally 0.001 to 10 mass % and preferably 0.01 to 5 mass % with respect to the solid content of the composition.

Hereinafter, the basic compound that can be preferably used in the actinic ray-sensitive or radiation-sensitive resin composition according to the embodiment of the present invention is described.

### Compound Having Structure Represented by Formula (A) to (E))

Examples of the basic compound include compounds having structures represented by Formulae (A) to (E).

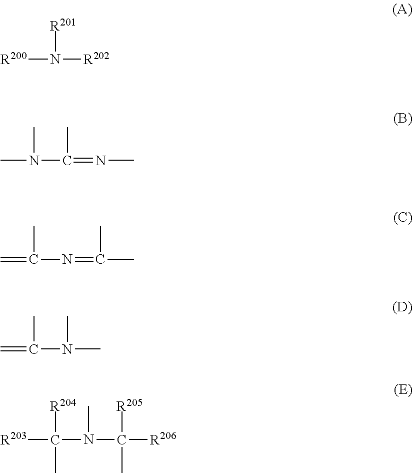

In Formulae (A) and (E),

R²⁰⁰, R²⁰¹, and R²⁰² may be identical to or different from each other, and represent hydrogen atoms, alkyl groups (preferably having 1 to 20 carbon atoms), cycloalkyl groups (preferably, having 3 to 20 carbon atoms), or aryl groups (having 6 to 20 carbon atoms). Here, R²⁰¹ and R²⁰² may be bonded to each other, so as to form a ring.

R²⁰³, R²⁰⁴, R²⁰⁵, and R²⁰⁶ may be identical to or different from each other, and represent an alkyl group having 1 to 20 carbon atoms.

With respect to the alkyl group, the alkyl group having the substituent is preferably an aminoalkyl group having 1 to 20 carbon atoms, a hydroxyalkyl group having 1 to 20 carbon atoms, or a cyanoalkyl group having 1 to 20 carbon atoms.

The alkyl groups in Formulae (A) and (E) are preferably unsubstituted.

Examples of the preferable compound include guanidine, aminopyrrolidine, pyrazole, pyrazoline, piperazine, aminomorpholine, aminoalkyl morpholine, or piperidine. Among these, examples of the more preferable compound include a compound having an imidazole structure, a diazabicyclo structure, an onium hydroxide structure, an onium carboxylate structure, a trialkylamine structure, an aniline structure, or a pyridine structure; an alkylamine derivative having a hydroxyl group and/or an ether bond; and an aniline derivative having a hydroxyl group and/or an ether bond.

Specific examples of the preferable compound include compounds disclosed in paragraph <0379> of US2012/0219913A1.

Preferable examples of the basic compound include an amine compound having a phenoxy group, an ammonium salt compound having a phenoxy group, an amine compound having a sulfonic acid ester group, and an ammonium salt compound having a sulfonic acid ester group.

These basic compounds may be used singly or two or more kinds thereof may be used in combination.

### Low Molecular Weight Compound That has Nitrogen Atom and has Group that Leaves Due to an Action of an Acid

As the basic compound, the low molecular weight compound that has a nitrogen atom and has a group that leaves due to an action of an acid (hereinafter, referred to as a “compound (C)”) is preferably used. The compound (C) is preferably an amine derivative having a group that is left due to an action of an acid on a nitrogen atom.

The group that is left due to an action of an acid is preferably an acetal group, a carbonate group, a carbamate group, a tertiary ester group, a tertiary hydroxyl group, and a hemiaminal ether group and more preferably a carbamate group and a hemiaminal ether group.

The molecular weight of the compound (C) is preferably 100 to 1000, more preferably 100 to 700, and even more preferably 100 to 500.

The compound (C) may have a carbamate group having a protective group on a nitrogen atom. The protective group constituting the carbamate group can be represented by Formula (d-1).

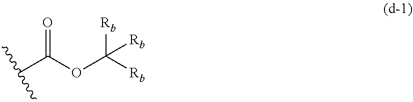

In Formula (d-1),

Rb's each independently represent a hydrogen atom, an alkyl group (preferably having I to 10 carbon atoms), a cycloalkyl group (preferably having 3 to 30 carbon atoms), an aryl group (preferably having 3 to 30 carbon atoms), an aralkyl group (preferably having 1 to 10 carbon atoms), or an alkoxyalkyl group (preferably having 1 to 10 carbon atoms). Rb's may be linked to each other to form a ring.

The alkyl group, the cycloalkyl group, the aryl group, and the aralkyl group represented by Rb may be substituted with a hydroxyl group, a cyano group, an amino group, a pyrrolidino group, a piperidino group, a morpholino group, an alkoxy group, or a halogen atom. The same is applied to the alkoxyalkyl group represented by Rb.

Rb is preferably a linear or branched alkyl group, a cycloalkyl group, or an aryl group. Rb is more preferably a linear or branched alkyl group or a cycloalkyl group.

Examples of the ring formed by linking two Rb's to each other include an alicyclic hydrocarbon group, an aromatic hydrocarbon group, a heterocyclic hydrocarbon group, or a derivative of these.

Examples of the specific structure of the group represented by Formula (d-1) include structures disclosed in paragraph <0466> of US2012/0135348A1, but the present invention is not limited thereto.

Among these, the compound (C) is preferably a compound represented by Formula(6).

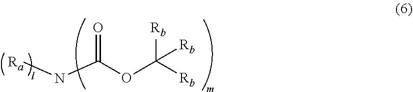

In Formula (6), Rₐ represents a hydrogen atom, an alkyl group, a cycloalkyl group, an aryl group, or an aralkyl group. In a case where 1 is 2, the two Rₐ's may be identical to or different from each other, and the two Rₐ's may be linked to each other to form a heterocyclic ring together with a nitrogen atom in the formula. The heterocyclic ring may include a hetero atom in addition to the nitrogen atom in the formula.

Rb has the same meaning as R_{b} in Formula (d-1), and preferable examples thereof are also the same.

l represents an integer of 0 to 2, m represents an integer of 1 to 3, and l+m=3 is satisfied.

In Formula (6), the alkyl group, the cycloalkyl group, the aryl group, and the aralkyl group as Rₐ in the may be substituted with the same groups as those described above as the groups that may be substituted with the alkyl group, the cycloalkyl group, the acyl group, and the aralkyl group as R_{b}.

Specific examples of the alkyl group, the cycloalkyl group, the aryl group, and the aralkyl group Rₐ in the (the alkyl group, the cycloalkyl group, the aryl group, and the aralkyl group may be substituted with the above groups) include the same groups described above as the specific examples for R_{b}.

According to the present invention, specific examples of the particularly preferable compound (C) include compounds disclosed in paragraph <0475> of US2012/0135348A1, but the present invention is not limited thereto.

The compound represented by Formula (6) can be synthesized based on JP2007-298569A and JP2009-199021A.

According to the present invention, the compound (C) having a group that is left due to an action of an acid on a nitrogen atom can be used singly or two or more kinds thereof may be used in a mixture.

### Basic Compound of Which Basicity Decreases or Disappears by Irradiation with an Actinic Ray or Radiation

A basic compound (hereinafter sometimes referred to as a “compound (PA)”) of which basicity decreases or disappears by irradiation with an actinic ray or radiation is a compound which has a proton acceptor functional group and is decomposed by irradiation with actinic rays or radiation and in which proton acceptor properties decrease or disappear or proton acceptor properties change to acidity.

The definition of the proton acceptor functional group is as described above.

According to the present invention, the acid dissociation constant pKa of the compound generated by decomposing the compound (PA) due to the irradiation with an actinic ray or radiation preferably satisfies pKa<−1, more preferably satisfies −13<pKa<−1, and even more preferably satisfies −13<pKa<−3.

According to the present invention, the acid dissociation constant pKa represents an acid dissociation constant pKa in an aqueous solution and is described in, for example, Chemical Handbook (II) (revised 4th edition, 1993, edited by The Chemical Society of Japan, Maruzen Co., Ltd.), and the lower the value, the higher the acid strength is. Specifically, the acid dissociation constant pKa in an aqueous solution can be actually measured by measuring an acid dissociation constant at 25° C. by using an infinitely diluted aqueous solution, and the Hammett's substituent constant and the value based on the database of values in the well-known documents can also be obtained by calculation by using a software package 1 below. All of the pKa values described in the present specification refer to values obtained by calculation by using this software package.

Software package 1: Advanced Chemistry Development (ACD/Labs) Software V8.14 for Solaris (1994-2007 ACD/Labs).

The compound (PA) generates, for example, a compound represented by the Formula (PA-1) as the proton adduct generated by the decomposition due to the irradiation with an actinic ray or radiation. The compound represented by Formula (PA-1) is a compound that has an acidic group together with a proton acceptor functional group such that the proton acceptor properties decrease or disappear compared with the compound (PA) or proton acceptor properties change to acidity.

Q-A-(X)ₙB-R (PA-1)

In Formula (PA-1),

Q represents —SO₃H, —CO₂H, or —W₁NHW₂R_{f}. Here, R_{f} represents an alkyl group (preferably having 1 to 20 carbon atoms), a cycloalkyl group (preferably having 3 to 20 carbon atoms), or an aryl group (preferably having 6 to 30 carbon atoms), and W₁ and W₂ each independently represent —SO₂— or —CO—.

A represents a single bond or a divalent linking group.

X represents —SO₂— or —CO—.

n represents 0 or 1.

B represents a single bond, an oxygen atom, or —N(Rₓ)R_{y}—. Here, Rₓ represents a hydrogen atom or a monovalent organic group, and R_{y} represents a single bond or a divalent organic group. Rₓ may be bonded to R_{y} to form a ring, and may be bonded to R to form a ring.

R represents a monovalent organic group having a proton acceptor functional group.

Formula (PA-1) is more specifically described.

The divalent linking group in A is preferably an alkylene group having at least one fluorine atom and more preferably a perfluoroalkylene group such as a perfluoroethylene group, a perfluoropropylene group, or a perfluorobutylene group.

Examples of the monovalent organic group in Rx include an alkyl group, a cycloalkyl group, an aryl group, an aralkyl group, and a resistance and alkenyl group, and these may further have a substituent.

The alkyl group in Rx is preferably a linear and branched alkyl group having 1 to 20 carbon atoms and may have an oxygen atom, a sulfur atom, or a nitrogen atom in an alkyl group.

The cycloalkyl group in Rx is preferably a monocyclic or polycyclic cycloalkyl group having 3 to 20 carbon atoms and may have an oxygen atom, a sulfur atom, or a nitrogen atom in the ring.

The aryl group in Rx is preferably an aryl group having 6 to 14 carbon atoms, and examples thereof include a phenyl group and a naphthyl group.

The aralkyl group in Rx is preferably an aralkyl group having 7 to 20 carbon atoms, and examples thereof include a benzyl group and a phenethyl group.

The alkenyl group in Rx is preferably 3 to 20 carbon atoms, and examples thereof include a vinyl group, an allyl group, and a styryl group.

Preferable examples of the divalent organic group in Ry include an alkylene group.

Examples of the ring structure that may be formed by bonding Rx and Ry to each other include a 5-membered to 10-membered ring including a nitrogen atom.

The proton acceptor functional group in R is as described above.

The organic group having this structure is preferably an organic group having 4 to 30 carbon atoms, and examples thereof include an alkyl group, a cycloalkyl group, an aryl group, an aralkyl group, and an alkenyl group.

In a case where B is —N(Rx)Ry-, it is preferable that R and Rx are bonded to each other to form a ring. The number of carbon atoms forming the ring is preferably 4 to 20, and the ring may be monocyclic or polycyclic and may contain an oxygen atom, a sulfur atom, or a nitrogen atom in the ring.

Examples of the monocyclic structure include a 4-membered to 8-membered ring including a nitrogen atom. Examples of the polycyclic structure include a structure completed by combining two or more monocyclic structures.

R_{f} in —W₁NHW₂R_{f} represented by Q is preferably a perfluoroalkyl group having 1 to 6 carbon atoms. At least one of W₁ or W₂ is preferably —SO₂—.

The compound (PA) is preferably an ionic compound. The proton acceptor functional group may be included in either an anion moiety or a cation moiety but is preferably included in in an anion moiety.

Preferable examples of the compound (PA) include compounds represented by Formulae (4) to (6).

R_{f}—W₂—N⁻—W₁-A-(X)ₙ—B—R[C]⁺ (4)

R—SO₃⁻[C]⁺ (5)

R—CO₂⁻[C]⁺ (6)

In Formulae (4) to (6), A, X, n, B, R, R_{f}, W₁ and W₂ have the same meaning as those in Formula (PA-1).

C⁺ indicates a counter cation.

The counter cation is preferably an onium cation. More specifically, preferable examples of the sulfonium cation described as S⁺(R₂₀₁)(R₂₀₂)(R₂₀₃) in Formula (ZI) in the acid generator include an iodonium cation described as I⁺(R₂₀₄)(R₂₀₅) in Formula (ZII).

Specific examples of the compound (PA) include compounds exemplified in paragraph <0280> of US2011/0269072A1.

### Onium Salt Which Becomes Relatively Weak Acid to Acid Generator

The actinic ray-sensitive or radiation-sensitive resin composition may contain onium salt which becomes a relatively weak acid to the acid generator as an acid diffusion control agent.

In the case where an acid generator and an onium salt which generates an acid which is a relatively weak acid compared with the acid generated from the acid generator are mixed to be used, in a case where an acid generated from the acid generator due to the irradiation with an actinic ray or radiation collides with an onium salt having an unreacted weak acid anion, a weak acid is left due to salt exchange to generate an onium salt having a strong acid anion. In this process, since a strong acid is exchanged with a weak acid having lower catalytic activity, so that the acid is apparently inactivated and the acid diffusion can be controlled.

The onium salt which becomes a relatively weak acid to the acid generator is preferably a compound represented by Formulae (d1-1) to (d1-3).

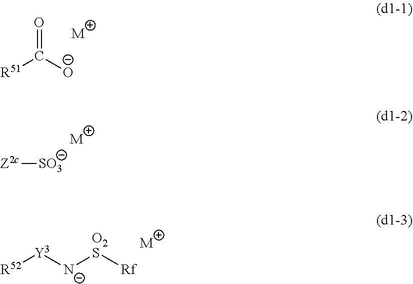

In the formula, R⁵¹ is a hydrocarbon group which may have a substituent, Z^{2c} is a hydrocarbon group having 1 to 30 carbon atoms which may have a substituent (it is assumed that a fluorine atom is not substituted for the carbon adjacent to S), R⁵² is an organic group, Y³ is a linear, branched, or cyclic alkylene group or arylene group, Rf is a hydrocarbon group including a fluorine atom, and M⁺'s each independently represent a sulfonium or iodonium cation.

Preferable examples of the sulfonium cation or the iodonium cation represented by M⁺ include sulfonium cations exemplified in Formula (ZI) and iodonium cations exemplified in Formula (ZII).

Preferable examples of the anion moiety of the compound represented by Formula (d1-1) include structures exemplified in paragraph [0198] of JP2012-242799A.

Preferable examples of the anion moiety of the compound represented by Formula (d1-2) include structures exemplified in paragraph [0201] of JP2012-242799A.

Preferable examples of the anion moiety of the compound represented by Formula (d1-3) include structures exemplified in paragraphs [0209] and [0210] of JP2012-242799A.

An onium salt which becomes a relatively weak acid compared with the acid generator may be a compound (C) (hereinafter, also referred to as a “compound (CA)”) which has a cation site and an anion site in the same molecule and in which the cation site and the anion site are linked by a covalent bond.

The compound (CA) is preferably a compound represented by any one of Formulae (C-1) to (C-3).

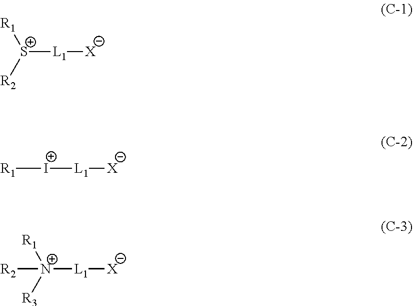

In Formulae (C-1) to (C-3),

R₁, R₂, and R₃ each represent a substituent having one or more carbon atoms.

L₁ represents a divalent linking group linking the cation site and the anion site or a single bond.

—X⁻ represent an anion site selected from —COO⁻, —SO₃⁻, —SO₂⁻, and —N⁻—R₄. R₄ represents a monovalent substituent having a carbonyl group: —C(═O)—, a sulfonyl group: —S(═O)₂—, and a sulfinyl group: —S(═O)— at a linking site to an adjacent N atom.

R₁, R₂, R₃, R₄, and L₁ may be bonded to each other to form a ring structure. In (C-3), two of R₁ to R₃ may be combined with each other to form a double bond with a N atom.

Examples of the substituent having 1 or more carbon atoms in R₁ to R₃ include an alkyl group, a cycloalkyl group, an aryl group, an alkyloxycarbonyl group, a cycloalkyloxycarbonyl group, an aryloxycarbonyl group, an alkylaminocarbonyl group, a cycloalkylaminocarbonyl group, and an arylaminocarbonyl group. The substituent is preferably an alkyl group, a cycloalkyl group, or an aryl group.

Examples of L₁ as the divalent linking group include a linear or branched alkylene group, a cycloalkylene group, an arylene group, a carbonyl group, an ether bond, an ester bond, an amide bond, a urethane bond, a urea bond, and a group obtained by combining two or more of these. L₁ is more preferably an alkylene group, an arylene group, an ether bond, an ester bond, or a group obtained by combining two or more of these.

Preferable examples of the compound represented by Formula (C-1) include compounds exemplified in paragraphs [0037] to [0039] of JP2013-006827A and paragraphs to [0029] of JP2013-008020A.

Preferable examples of the compound represented by Formula (C-2) include compounds exemplified in paragraphs [0012] and [0013] of JP2012-189977A.

Preferable examples of the compound represented by Formula (C-3) include compounds exemplified in paragraphs [0029] to [0031] of JP2012-252124A.

### Hydrophobic Resin

The composition according to the embodiment of the present invention may contain a hydrophobic resin (HR). The hydrophobic resin (HR) is preferably different from the resin (preferably the resin (A)).

It is preferable that the hydrophobic resin (HR) is designed to be unevenly distributed on the interface, but, differently from the surfactant, a hydrophilic group does not need to be included in the molecule and may not contribute to the even mixture of the polar/non-polar materials.

Examples of the effect of adding the hydrophobic resin include control a static/dynamic contact angle of a resist film surface against water, improvement of the immersion liquid followability, and the suppression of outgas.

In view of the uneven distribution on the film surface, the hydrophobic resin (HR) preferably includes one or more kinds of any of a “fluorine atom”, a “silicon atom”, or a “CH₃ substructure contained in a side chain portion of the resin” and more preferably includes two or more kinds thereof.

In a case where the hydrophobic resin (HR) includes a fluorine atom and/or a silicon atom, the fluorine atom and/or the silicon atom in the hydrophobic resin (HR) may be included in the main chain of the resin and may be included in the side chain.

In the case where the hydrophobic resin (HR) includes a fluorine atom, the partial structure having a fluorine atom is preferably a resin having an alkyl group having a fluorine atom, a cycloalkyl group having a fluorine atom, or an aryl group having a fluorine atom.

The alkyl group (preferably having 1 to 10 carbon atoms and more preferably having 1 to 4 carbon atoms) having a fluorine atom is a linear or branched alkyl group in which at least one hydrogen atom is substituted with a fluorine atom and may further have a substituent in addition to the fluorine atom.

The cycloalkyl group having a fluorine atom and the aryl group having a fluorine atom are respectively a cycloalkyl group in which one hydrogen atom is substituted with a fluorine atom and an aryl group having a fluorine atom, and may further have a substituent in addition to the fluorine atom.

Preferable examples of the alkyl group having a fluorine atom, the cycloalkyl group having a fluorine atom, and the aryl group having a fluorine atom include groups represented by the Formulae (F2) to (F4), but the present invention is not limited to these.

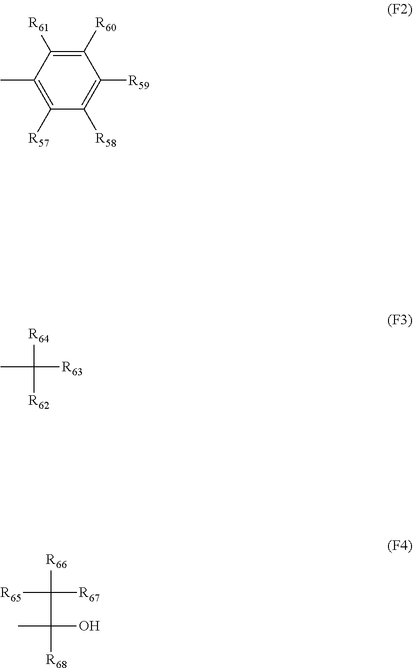

In Formulae (F2) to (F4),

R₅₇ to R₆₈ each independently represent a hydrogen atom, a fluorine atom, or a (linear or branched) alkyl group. At least one of R₅₇, . . . , or R₆₁, at least one of R₆₂, . . . or R₆₄, and at least one of R₆₅, . . . , or R₆₈ each independently represent a fluorine atom or an alkyl group in which at least one hydrogen atom is substituted with a fluorine atom (preferably having 1 to 4 carbon atoms).

All of R₅₇ to R₆₁ and R₆₅ to R₆₇ are preferably fluorine atoms. R₆₂, R₆₃, and R₆₈ are preferably an alkyl group (preferably having 1 to 4 carbon atoms) at least one hydrogen atom is substituted with a fluorine atom and more preferably a perfluoroalkyl group having 1 to 4 carbon atoms. R₆₂ and R₆₃ may be linked to each other to form a ring.

The hydrophobic resin (HR) may contain a silicon atom. A partial structure having a silicon atom is preferably a resin having an alkylsilyl structure (preferably a trialkylsilyl group) or a cyclic siloxane structure.

Examples of the repeating units having a fluorine atom or a silicon atom include repeating units exemplified in [0519] of US2012/0251948A1.

As described above, it is also preferable that the hydrophobic resin (HR) includes a CH₃ partial structure in the side chain moiety.

Here, the CH₃ partial structure of the side chain moiety in the hydrophobic resin (HR) (hereinafter also simply referred to as a “side chain CH₃ partial structure”) includes the CH₃ partial structure included in the ethyl group, the propyl group, or the like.

Meanwhile, a methyl group directly bonded to the main chain of the hydrophobic resin (HR) (for example, an α-methyl group of a repeating unit having a methacrylic acid structure) is not included in the CH₃ partial structure of the present invention because contribution to uneven distribution on the surface of the hydrophobic resin (HR) is small due to the influence of the main chain.

More specifically, this is a case where the hydrophobic resin (HR) is a repeating unit derived from a monomer having a polymerizable site having a carbon-carbon double bond, such as a repeating unit represented by Formula (M), and in a case where R₁₁ to R₁₄ are CH₃ “itself”, CH₃ is not included in the CH₃ partial structure of the side chain moiety of the present invention.

Meanwhile, the CH₃ partial structure that is present from the C—C main chain via a certain atom corresponds to the CH₃ partial structure of the present invention. For example, in a case where R₁₁ is an ethyl group (CH₂CH₃), one CH₃ partial structure of the present invention is provided.

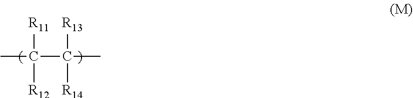

In Formula (M),

R₁₁ to R₁₄ each independently represent a side chain moiety.

Examples of R₁₁ to R₁₄ of the side chain moiety include a hydrogen atom and a monovalent organic group.

Examples of the monovalent organic group of R₁₁ to R₁₄ include an alkyl group, a cycloalkyl group, an acyl group, an alkyloxycarbonyl group, a cycloalkyloxycarbonyl group, an aryloxycarbonyl group, an alkyl aminocarbonyl group, a cycloalkylaminocarbonyl group, and an arylaminocarbonyl group, and these groups may further have a substituent.

The hydrophobic resin (HR) is preferably a resin having a repeating unit having a CH₃ partial structure in a side chain moiety and more preferably has at least one repeating unit (x) of a repeating unit represented by Formula (II) or a repeating unit represented by Formula (III), as such a repeating unit.

Hereinafter, the repeating unit represented by Formula (II) will be described in detail.

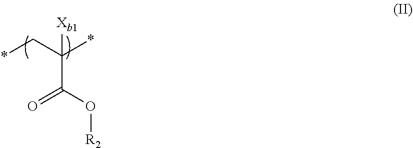

In Formula (II), X_{b1} represents a hydrogen atom, an alkyl group, a cyano group, or a halogen atom, and R₂ represents an organic group that has one or more CH₃ partial structures and that is stable to an acid. Here, more specifically, the organic group which is stable to an acid is preferably an organic group that does not have an acid-decomposable group (a group that is decomposed due to an action of an acid and generates a polar group such as a carboxy group).

The alkyl group of X_{b1} is preferably an alkyl group having 1 to 4 carbon atoms, and examples thereof include a methyl group, an ethyl group, a propyl group, a hydroxymethyl group, or a trifluoromethyl group, but a methyl group is more preferable.

X_{b1} is preferably a hydrogen atom or a methyl group.

Examples of R₂ include an alkyl group, a cycloalkyl group, an alkenyl group, a cycloalkenyl group, an aryl group, and an aralkyl group each having one or more CH₃ partial structures. The cycloalkyl group, the alkenyl group, the cycloalkenyl group, the aryl group, and the aralkyl group each may further have an alkyl group as a substituent.

R₂ is preferably an alkyl group or an alkyl-substituted cycloalkyl group each having one or more CH₃ partial structures.

The organic group as R₂ that has one or more CH₃ partial structures and that is stable to acid preferably has 2 to 10 CH₃ partial structures and more preferably 2 to 8 CH₃ partial structures.

Preferably specific examples of the repeating unit represented by Formula (II) are provided below. The present invention is not limited thereto.

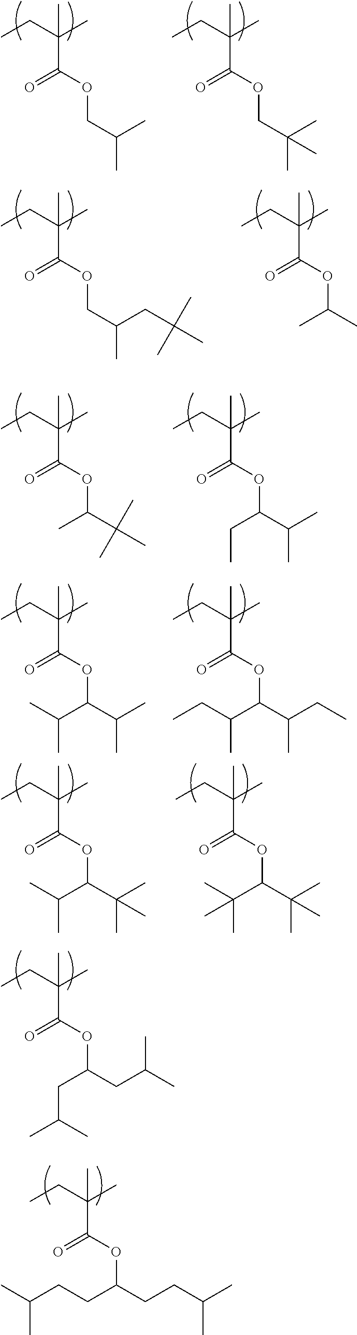

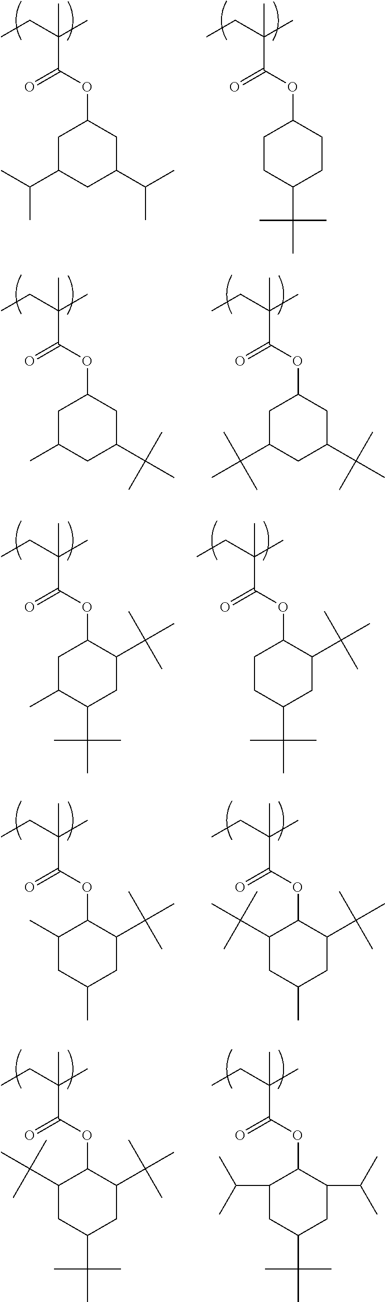

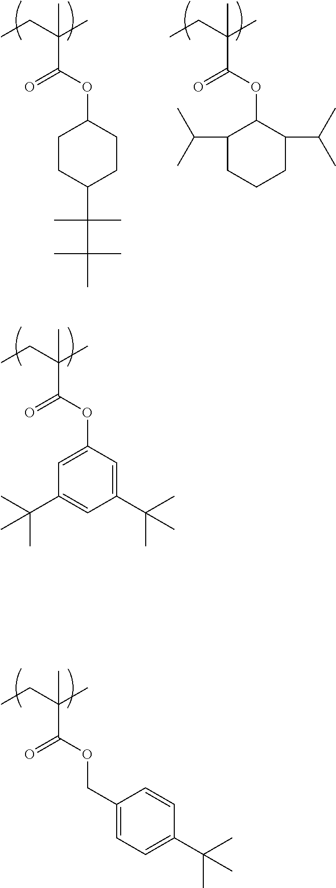

The repeating unit represented by Formula (II) is preferably a repeating unit that is stable to acid (non-acid decomposable). Specifically, it is preferable that the repeating unit is a repeating unit not having a group that is decomposed due to an action of an acid and generates a polar group.

Hereinafter, the repeating unit represented by Formula (III) will be described in detail.

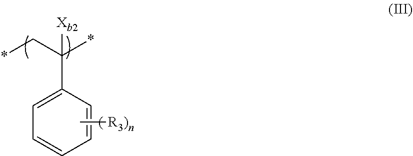

In Formula (III), X_{b2} represents a hydrogen atom, an alkyl group, a cyano group, or a halogen atom, R₃ represents an organic group that has one or more CH₃ partial structures and that is stable to acid, and n is an integer of 1 to 5.

The alkyl group of X_{b2} is preferably an alkyl group having 1 to 4 carbon atoms, and examples thereof include a methyl group, an ethyl group, a propyl group, a hydroxymethyl group, and a trifluoromethyl group, but a hydrogen atom is preferable.

X_{b2} is preferably a hydrogen atom.

Since R₃ is an organic group that is stable to acid, more specifically, the organic group that does not have an acid-decomposable group is preferable.

Examples of R₃ include an alkyl group having one or more CH₃ partial structures.

The organic group as R₃ that has one or more CH₃ partial structures and that is stable to an acid preferably has 1 to 10 CH₃ partial structures, more preferably 1 to 8 CH₃ partial structures, and even more preferably 1 to 4 CH₃ partial structures.

n represents an integer of 1 to 5, more preferably represents an integer of 1 to 3, and even more preferably represents 1 or 2.

Preferably specific examples of the repeating unit represented by Formula (III) are provided below. The present invention is not limited thereto.

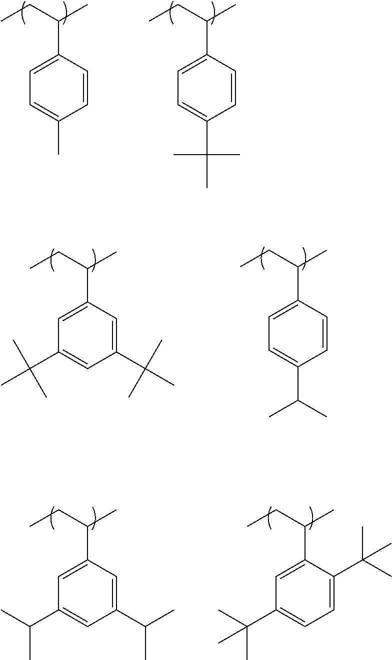

The repeating unit represented by Formula (III) is preferably a repeating unit that is stable to acid (non-acid decomposable). Specifically, it is preferable that the repeating unit is a repeating unit not having a group that is decomposed due to an action of an acid and generates a polar group.

In the case where the hydrophobic resin (HR) includes a CH₃ partial structure in the side chain moiety and particularly does not have a fluorine atom and a silicon atom, the content of the at least one repeating unit (x) of the repeating unit represented Formula (II) or the repeating unit represented by Formula (III) is preferably 90 mol % or more and more preferably 95 mol % or more with respect to the all repeating units of the hydrophobic resin (HR). The content is generally 100 mol % or less with respect to the all repeating units of the hydrophobic resin (HR).

In a case where the hydrophobic resin (HR) contains 90 mol % or more of the at least one repeating unit (x) of the repeating unit represented Formula (II) or the repeating unit represented Formula (III) with respect to the all repeating units of the hydrophobic resin (HR), the surface free energy of the hydrophobic resin (HR) increases. As a result, the hydrophobic resin (HR) is hardly unevenly distributed on the surface of the resist film, and a static/dynamic contact angle of the resist film against water is securely improved, so as to improve immersion liquid followability.

Even in a case where (i) a fluorine atom and/or a silicon atom is included or in a case where (ii) a CH₃ partial structure is included in a side chain moiety, the hydrophobic resin (HR) may have at least one group selected from the group of (x) to (z) as below:

(x) an acid group;

(y) a group having a lactone structure, an acid anhydride group, or an acid imide group; and

(Z) a group that is decomposed due to an action of an acid.

Examples of the acid group (x) include a phenolic hydroxyl group, a carboxylic acid group, a fluorinated alcohol group, a sulfonic acid group, a sulfonamide group, a sulfonylimide group, an (alkylsulfonyl) (alkylcarbonyl) methylene group, an (alkylsulfonyl) (alkylcarbonyl) imide group, a bis(alkylcarbonyl) methylene group, a bis(alkylcarbonyl) imide group, a bis(alkylsulfonyl) methylene group, a bis(alkylsulfonyl) imide group, a tris(alkylcarbonyl group) methylene group, and a tris(alkylsulfonyl) methylene group.

Preferable examples of the acid group include a fluorinated alcohol group (preferably hexafluoroisopropanol), a sulfonimide group, or a bis(alkylcarbonyl group) methylene group.

Examples of the repeating unit having an acid group (x) include a repeating unit in which an acid group is directly bonded to the main chain of the resin like a repeating unit of acrylic acid or methacrylic acid and a repeating unit in which an acid group is bonded to the main chain of the resin via a linking group, a polymerization initiator having an acid group or a chain transfer agent can be introduced to a terminal of a polymer chain in a case of polymerization, and all cases are preferable. The repeating unit having the acid group (x) may have at least one of a fluorine atom or a silicon atom.

The content of the repeating unit having the acid group (X) is preferably 1 to 50 mol %, more preferably 3 to 35 mol %, and even more preferably 5 to 20 mol % with respect to all repeating units in the hydrophobic resin (HR).

Specific examples of the repeating unit having the acid group (x) are shown below, but the present invention is not limited thereto. In the formula, Rx represents a hydrogen atom, CH₃, CF₃, or CH₂OH.

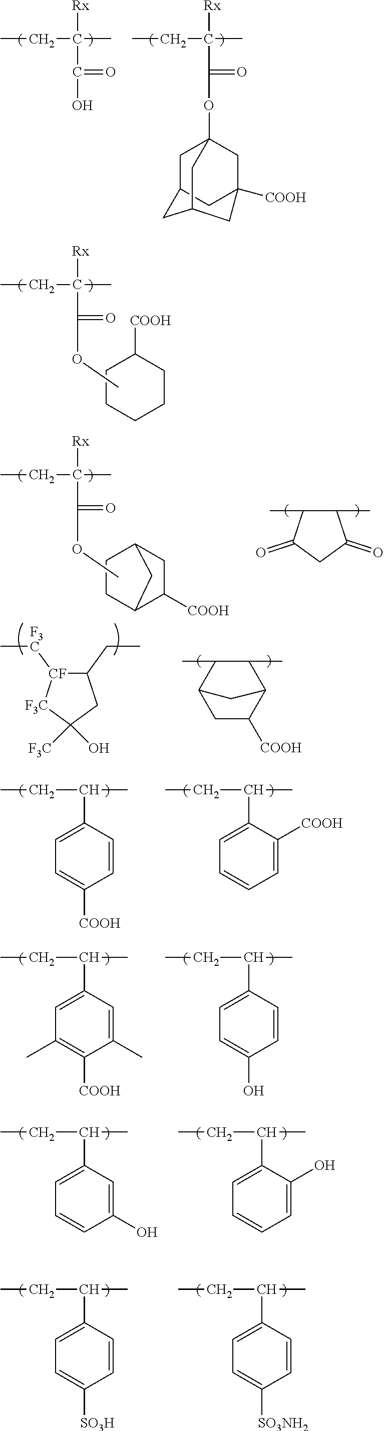

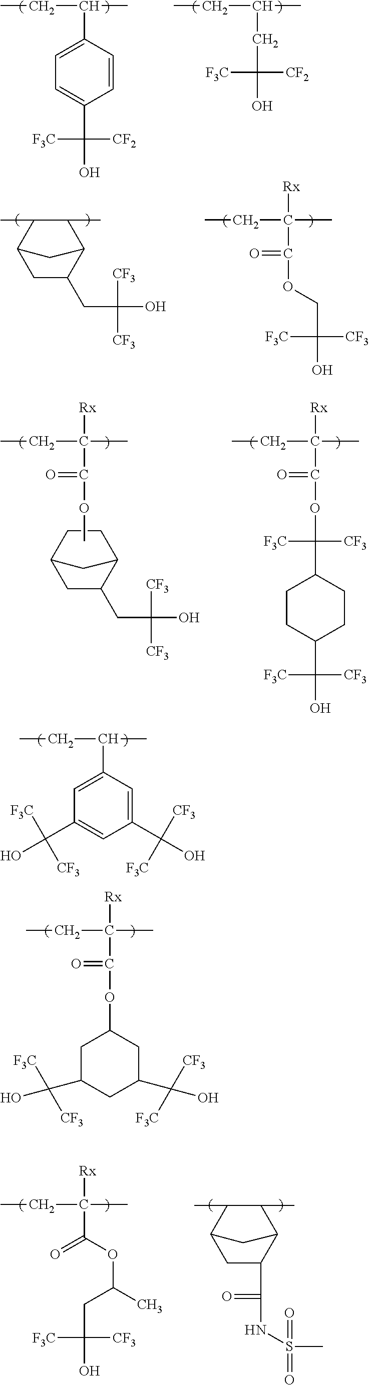

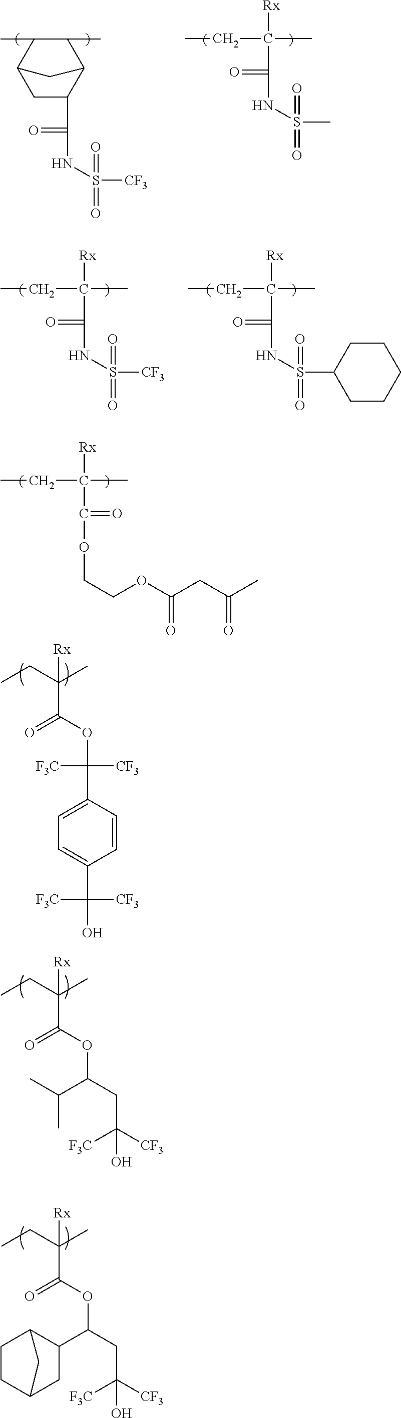

The group having a lactone structure, the acid anhydride group, or the acid imide group (y) are particularly preferably a group having a lactone structure.

For example, the repeating unit including these groups is a repeating unit in which this group is directly bonded to a main chain of a resin such as a repeating unit of acrylic acid ester and methacrylic acid ester. The repeating unit may be a repeating unit in which this group is bonded to a main chain of a resin via a linking group. This repeating unit may be introduced at a terminal of a resin by using a polymerization initiator or a chain transfer agent having this group at the time of polymerization.

Examples of the repeating unit having a group having a lactone structure include repeating units which are the same as the repeating units having a lactone structure described in the section of the resin (A) above.

The content of the repeating unit having the group having a lactone structure, the acid anhydride group, or the acid imide group is preferably 1 to 100 mol %, more preferably 3 to 98 mol %, and still more preferably 5 to 95 mol % based on the all repeating units in the hydrophobic resin (HR).

Examples of the repeating unit having the group (z) which is decomposed by the action of an acid in the hydrophobic resin (HR) include repeating units which are the same as the repeating units having an acid-decomposable group as provided as the resin (A). The repeating unit having the group (z) which is decomposed by the action of an acid may have at least one of a fluorine atom or a silicon atom. With respect to the hydrophobic resin (HR), the content of the repeating unit having a group (z) which is decomposed due to an action of an acid is preferably 1 to 80 mol %, more preferably 10 to 80 mol %, and even more preferably 20 to 60 mol % with respect to all repeating units in the resin (HR).

The hydrophobic resin (HR) may further have a repeating unit different from the repeating unit described above.

The repeating unit containing a fluorine atom is preferably 10 to 100 mol % and more preferably 30 to 100 mol % with respect to the all repeating units included in the hydrophobic resin (HR). The repeating unit including a silicon atom is preferably 10 to 100 mol % and more preferably 20 to 100 mol % with respect to the all repeating units included in the hydrophobic resin (HR).

Meanwhile, particularly, in a case where the hydrophobic resin (HR) includes a CH₃ partial structure in a side chain moiety, an aspect in which the hydrophobic resin (HR) does not substantially contain a fluorine atom and a silicon atom is also preferable. Also, it is preferable that the hydrophobic resin (HR) is substantially constituted only by repeating units constituted only by atoms selected from a carbon atom, an oxygen atom, a hydrogen atom, a nitrogen atom, and a sulfur atom.

The weight-average molecular weight of the hydrophobic resin (D) in terms of standard polystyrene is preferably in the range of 1,000 to 100,000 and more preferably in the range of 1,000 to 50,000.

The content of the hydrophobic resin (HR) in the composition is preferably 0.01 to 10 mass % and more preferably 0.05 to 8 mass % with respect to the total solid content in the composition according to the embodiment of the present invention.

The hydrophobic resin (HR) may be used singly and two or more kinds thereof may be used in combination. In a case where two or more hydrophobic resins (HR) are used in combination, it is preferable that the total content is in the above range.

In the hydrophobic resin (HR), the content of the residual monomer or oligomer components is preferably 0.01 to 5 mass % and more preferably 0.01 to 3 mass %. The molecular weight distribution (Mw/Mn, hereinafter, also referred to as a “dispersion degree”) is preferably in the range of 1 to 5 and more preferably in the range of 1 to 3.

As the hydrophobic resin (HR), various kinds of commercially available products may be used, or the hydrophobic resin (HR) may be synthesized by a general method (for example, radical polymerization).

### Solvent

The composition according to the embodiment of the present invention generally contains a solvent.

Examples of the solvent that can be used in preparing the composition include organic solvents such as alkylene glycol monoalkyl ether carboxylate, alkylene glycol monoalkyl ether, lactic acid alkyl ester, alkyl alkoxypropionate, cyclic lactone (preferably having 4 to 10 carbon atoms), a monoketone compound which may have a ring (preferably having 4 to 10 carbon atoms), alkylene carbonate, alkyl alkoxyacetate, and alkyl pyruvate.

Specific examples of these solvents include solvents disclosed in paragraphs <0441> to <0455> of US2008/0187860A.

According to the present invention, a mixed solvent obtained by mixing a solvent containing a hydroxyl group in a structure and a solvent not containing a hydroxyl group may be used as the organic solvent.

As the solvent containing a hydroxyl group in a structure and the solvent not containing a hydroxyl group, the example compounds described above can be appropriately selected, but the solvent containing a hydroxyl group is preferably alkylene glycol monoalkyl ether and lactatic acid alkyl ester and more preferably propylene glycol monomethyl ether (PGME, also known as 1-methoxy-2-propanol), methyl 2-hydroxyisobutyrate, and ethyl lactate. As the solvent not containing a hydroxyl group, alkylene glycol monoalkyl ether acetate, alkyl alkoxy propionate, a monoketone compound that may contain a ring, cyclic lactone, and alkyl acetate are preferable. Among these, propylene glycol monomethyl ether acetate (PGMEA, also referred to as 1-methoxy-2-acetoxypropane), ethyl ethoxypropionate, 2-heptanone, γ-butyrolactone, cyclohexanone, and butyl acetate are more preferable, and propylene glycol monomethyl ether acetate, ethyl ethoxypropionate, and 2-heptanone are even more preferable.

The mixing ratio (mass) of the solvent containing a hydroxyl group and the solvent not containing a hydroxyl group is 1/99 to 99/1, preferably 10/90 to 90/10, and more preferably 20/80 to 60/40. A mixed solvent containing 50 mass % or more of the solvent not containing a hydroxyl group is particularly preferable in view of coating uniformity

The solvent preferably includes propylene glycol monomethyl ether acetate and is preferably a single solvent of propylene glycol monomethyl ether acetate or a mixed solvent of two or more kinds containing propylene glycol monomethyl ether acetate.

### Other Additives

### Surfactant

The composition according to the embodiment of the present invention may not further contain a surfactant, but in a case where the composition contains a surfactant, and a fluorine-based and/or silicon-based surfactant (fluorine-based surfactant, silicon-based surfactant, and surfactant having both of fluorine atom and silicon atom) are preferable.

In a case where the composition according to the embodiment of the present invention contains a surfactant, in a case where an exposure light source having 250 nm or lower, particularly 220 nm or lower is used, a resist pattern having excellent adhesiveness and fewer development defects can be applied at favorable sensitivity and resolutions.

Examples of the fluorine-based and/or silicon-based surfactants include surfactants disclosed in paragraph <0276> of US2008/0248425A.

According to the present invention, surfactants other than fluorine-based and/or silicon-based surfactants disclosed in paragraph <0280> of US2008/0248425A can be used.

These surfactants may be used singly, or several kinds thereof may be used in combination.

In a case where the composition according to the embodiment of the present invention contains a surfactant, the used amount of the surfactant is preferably 0.0001 to 2 mass % and more preferably in the range of 0.0005 to 1 mass % with respect to the total solid content of the composition.

Meanwhile, in a case where the addition amount of the surfactant is 10 ppm or lower with respect to the total amount (except for the solvent) of the composition, the uneven distribution properties of the surface of the hydrophobic resin increase, and accordingly, the resist film surface can become more hydrophobic, and thus it is possible to improve water followability in a case of immersion exposure.

### Carboxylic Acid Onium Salt

The composition according to the embodiment of the present invention may or may not contain carboxylic acid onium salt. Examples of the carboxylic acid onium salt include salts disclosed in paragraphs <0605> and <0606> of US2008/0187860A.

These carboxylic acid onium salts can be synthesized by reacting sulfonium hydroxide, iodonium hydroxide, ammonium hydroxide, and carboxylic acid with silver oxide in an appropriate solvent.

In a case where the composition according to the embodiment of the present invention contains carboxylic acid onium salt, the content thereof is generally 0.1 to 20 mass %, preferably 0.5 to 10 mass %, and more preferably 1 to 7 mass % with respect to the total solid content of the composition.

### Other Additives

In the composition according to the embodiment of the present invention, if necessary, an acid proliferation agent, a dye, a plasticizer, a photosensitizer, a light absorber, an alkali-soluble resin, a dissolution inhibitor, a compound (for example, a phenol compound having a molecular weight of 1,000 or lower, alicyclic or aliphatic compound having a carboxy group) that promotes solubility in the developer, and the like can be further contained.

The phenol compound having a molecular weight of 1000 or less can be easily synthesized by those skilled in the art, for example, with reference to methods disclosed in JP1992-122938A (JP-H04-122938A), JP1990-028531A (JP-H02-028531A), U.S. Pat. No. 4,916,210A, and EP219294B.

Specific examples of alicyclic or aliphatic compounds having a carboxy group include a carboxylic acid derivative that has a steroid structure such as cholic acid, deoxycholic acid, and lithocholic acid, an adamamanecarboxylic acid derivative, adamantanedicarboxylic acid, cyclohexanecarboxylic acid, and cyclohexane dicarboxylic acid.

### Preparation Method

In view of resolving power improvement, with the composition according to the embodiment of the present invention, it is preferable to form a resist film having a film thickness of 90 nm or less and preferably having a film thickness of 85 nm or less. In a case where coatability and film formability are improved by setting the concentration of the solid. content in the composition to an appropriate range such that the composition has an appropriate viscosity, the film thickness can be obtained.

The concentration of the solid content of the composition according to the embodiment of the present invention is generally 1.0 to 10 mass %, preferably 2.0 to 5.7 mass %, and more preferably 2.0 to 5.3 mass %. By causing the solid content concentration to be in the above range, the substrate can be uniformly coated with the resist solution, and also an excellent resist pattern can be formed by LWR. Although the reason is unclear, it is considered that, by setting the concentration of the solid content to 10 mass % or less and preferably 5.7 mass % or less, the aggregation of the material, particularly, the acid generator in the resist solution is suppressed, and as a result, a uniform resist film can be formed.

The concentration of the solid content is a mass percentage of a mass of the other resist components excluding the solvent with respect to the total mass of the composition.

The composition according to the embodiment of the present invention can be used by dissolving the above components in a predetermined organic solvent, preferably the mixed solvent, performing filter filtration, and applying on a predetermined support (substrate). The pore size of the filter used in the filter filtration is 0.1 μm or lower, more preferably 0.05 μm or lower, and even more preferably 0.03 μm or lower, and preferably made of polytetrafluoroethylene, polyethylene, or nylon. In the filter filtration, for example, as disclosed in JP2002-062667A, cyclical filtration may be performed, or filtration may be performed by connecting plural kinds of filters in series or in parallel. In addition, the composition may be filtrated a plurality of times. Before or after the filter filtration, a deaeration treatment or the like may be performed on the composition.

### Application

The composition according to the embodiment of the present invention relates to an actinic ray-sensitive or radiation-sensitive resin composition of which properties change in reaction to the irradiation with an actinic ray or radiation. More specifically, the present invention relates to an actinic ray-sensitive or radiation-sensitive resin composition used in a step of manufacturing a semiconductor such as an IC, the manufacturing of a circuit board such as a liquid crystal and a thermal head, the manufacturing of a mold structure for imprinting, a photofabrication step, a lithographic printing plate, and an acid curable composition.

### Pattern Forming Method

The present invention also relates to a pattern forming method using the actinic ray-sensitive or radiation-sensitive resin composition. Subsequently, the pattern forming method according to the embodiment of the present invention is described. Together with the description of the pattern forming method, the resist film according to the embodiment of the present invention is described.

The pattern forming method according to the embodiment of the present invention includes:

(i) a resist film forming step of forming a resist film by using the actinic ray-sensitive or radiation-sensitive resin composition;

(ii) an exposure step of exposing the resist film; and

(iii) a development step of developing the exposed resist film with a developer.

The pattern forming method according to the embodiment of the present invention is not particularly limited, as long as the method includes the steps of (i) to (iii) and may further have the following steps.

In the pattern forming method according to the embodiment of the present invention, the exposure method in (ii) the exposure step is preferably immersion exposure.

The pattern forming method according to the embodiment of the present invention preferably includes (iv) a prebaking step before (ii) the exposure step.

The pattern forming method according to the embodiment of the present invention preferably includes (v) a postexposure baking step after (ii) the exposure step.

The pattern forming method according to the embodiment of the present invention may include (ii) the exposure step a plurality of times.

The pattern forming method according to the embodiment of the present invention may include (iv) the prebaking step a plurality of times.

The pattern forming method according to the embodiment of the present invention may include (v) the postexposure baking step a plurality of times.

The resist film according to the embodiment of the present invention is a film formed of the actinic ray-sensitive or radiation-sensitive resin composition, and is preferably a film formed by coating a substrate with the composition, specifically.

In the pattern forming method according to the embodiment of the present invention, (i) the resist film forming step, (ii) the exposure step, and (iii) the development step can be performed by generally known methods.

If necessary, an antireflection film may be formed between a resist film and a substrate. As the antireflection film, well-known organic and inorganic antireflection films can be appropriately used.

The substrate is not particularly limited, and a substrate that is generally used in a step of manufacturing a semiconductor such as IC, a step of manufacturing a circuit board of a liquid crystal, a thermal head, or the like, a lithography step of photo fabrication, or the like can be used, and specific examples thereof include an inorganic substrate such as silicon, SiO₂, or SiN or a coating type inorganic substrate such as Spin On Glass (SOG).

As described above, the pattern forming method according to the embodiment of the present invention preferably includes (iv) a prebaking (PB) step after (i) the resist film forming step and before (ii) the exposure step.

It is preferable to include v) the postexposure baking (PEB) step after (ii) the exposure step and before (iii) the development step.

The baking as described above accelerates the reaction of the exposed portion and improves sensitivity and/or pattern profile.

In both cases of PB and PEB, the heating temperature is preferably 70° C. to 130° C. and more preferably 80° C. to 120° C.

In both cases of PB and PEB, the heating time is preferably 30 to 300 seconds, more preferably 30 to 180 seconds, and even more preferably 30 to 90 seconds.

The heating can be performed by means included in a general exposure machine and a general developing machine and may be performed by using a hot plate or the like.

There is no limitation on the light source wavelength used in the exposure device, but examples thereof include infrared light, visible light, ultraviolet light, far ultraviolet light, extreme ultraviolet light, X-rays, or electron beams, and far ultraviolet light having a wavelength of preferably 250 nm or less, more preferably 220 nm or less, and even more preferably 1 to 200 nm, specific examples thereof include KrF excimer laser (248 nm), ArF excimer laser (193 nm), F₂ excimer laser (157 nm), X-rays, EUV (13 nm), and electron beams, KrF excimer laser, ArF excimer laser, EUV, or electron beams is preferable, and an ArF excimer laser is more preferable.

In the pattern forming method according to the embodiment of the present invention, in the exposure step (ii), an immersion exposure method can be applied. The immersion exposure method can be combined with super-resolution techniques such as a phase shift method and a deformed illumination method. The immersion exposure can be performed, for example, by a method described in paragraphs <0594> to <0601> of JP2013-242397A.

In a case where the receding contact angle of the resist film formed by using the composition according to the embodiment of the present invention is too small, the composition cannot be suitably used in the case where exposure is performed through an immersion medium, and the effect of reducing water residue (watermark) defects cannot be sufficiently exhibited. In order to realize a preferable receding contact angle, it is preferable to cause the hydrophobic resin (HR) to be included in the composition. Otherwise, an immersion liquid-hardly soluble film (hereinafter also referred to as a “topcoat”) formed by the hydrophobic resin (HR) may be provided on the upper surface of the resist film. Examples of the functions necessary for the topcoat include coating suitability to an upper layer portion of the resist film or hardly soluble properties in the immersion liquid. It is preferable that the composition for forming a topcoat is not mixed with a composition film formed of the composition according to the embodiment of the present invention and can be evenly applied to an upper layer of the composition film formed of the composition according to the embodiment of the present invention.

The preparation of the composition for forming the topcoat and the method of forming the topcoat are not particularly limited, and can be performed based on methods well-known in the related art, for example, the description disclosed in paragraphs <0072> to <0082> of JP2014-059543A.

In (iii) the development step described below, in a case of using a developer containing an organic solvent, it is preferable to form a topcoat containing a basic compound disclosed in JP2013-061648A on a resist film.

Even in a case where the exposure is performed by a method other than the immersion exposure method, a topcoat may be formed on the resist film.

In the immersion exposure step, an exposure head scans a wafer at a high speed, forms an exposure pattern, and an immersion liquid is required to move on the wafer along the movement of the formation of the exposure pattern. Therefore, the contact angle of the immersion liquid to the resist film in a dynamic state becomes important, and thus the resist requires a performance that follows to the high speed scanning of the exposure head without remaining the liquid droplet.

In (iii) the development step, it is preferable to use the developer (hereinafter, also referred to as an organic developer) containing an organic solvent.

As an organic developer, a polar solvent such as a ketone-based solvent, an ester-based solvent, an alcohol-based solvent, an amide-based solvent, and an ether-based solvent or a hydrocarbon-based solvent can be used.

The plurality of kinds of the solvents may be mixed or may be mixed with a solvent other than the above or water. In order to sufficiently obtain the effect of the present invention, the moisture content of the developer as a whole is preferably less than 10 mass %, and it is more preferable that substantially no moisture is contained.

That is, the content of the organic solvent with respect to the organic developer is preferably 90 mass % to 100 mass % and more preferably 95 mass % to 100 mass % with respect to the total amount of the developer.

Particularly, the organic developer is preferably a developer containing at least one kind of organic solvent selected from the group consisting of a ketone-based solvent, an ester-based solvent, an alcohol-based solvent, an amide-based solvent, and an ether-based solvent.

The vapor pressure of the organic developer is preferably 5 kPa or lower, more preferably 3 kPa or lower, and even more preferably 2 kPa or lower at 20° C. In a case where the vapor pressure of the organic developer is 5 kPa or lower, the evaporation of the developer on the substrate or in a development cup is suppressed, and thus the temperature uniformity in the wafer surface increases, and as a result, the dimension uniformity in the wafer surface improves.

An appropriate amount of a surfactant can be added to the organic developer, if necessary.

The surfactant is not particularly limited but, for example, ionic or nonionic fluorine and/or silicon-based surfactants and the like can be used. Examples of the fluorine-based and/or silicon-based surfactants include surfactants disclosed in JP1987-036663A (JP-S62-036663A), JP1986-226746A (JP-S61-226746A), JP1986-226745A (JP-S61-226745A), JP1987-170950A (JP-S62-170950A), JP1988-034540A (JP-S63-034540A), JP1995-230165A (JP-H07-230165A), JP1996-062834A (JP-H08-062834A), JP1997-054432A (JP-H09-054432A), JP1997-005988A (JP-H09-005988A), U.S. Pat. No. 5,405,720A, U.S. Pat. No. 5,360,692A, U.S. Pat. No. 5,529,881 A, U.S. Pat. No. 5,296,330A, U.S. Pat. No. 5,436,098A, U.S. Pat. No. 5,576,143A, U.S. Pat. No. 5,294,511A, and U.S. Pat. No. 5,824,451A, and a nonionic surfactant is preferable. The nonionic surfactant is not particularly limited, but it is more preferable to use a fluorine-based surfactant or a silicon-based surfactant.

The usage amount of the surfactant is generally 0.001 to 5 mass %, preferably 0.005 to 2 mass %, and more preferably 0.01 to 0.5 mass % with respect to the total amount of the developer.

The organic developer may include a basic compound. Examples of the basic compound include an amine compound, an amide group-containing compound, a urea compound, and a nitrogen-containing heterocyclic compound.

As the developing method, for example, a method of immersing a substrate in a tank filled with a developer for a predetermined period of time (dipping method), a developing method by raising the developer on the surface of a substrate by surface tension and causing the developer to stand for a certain period of time (puddle method), a method of spraying a developer to the surface of a substrate (spraying method), and a method of continuously jetting a developer while scanning a developer jetting nozzle at a constant speed on a substrate rotating at a constant speed (dynamic dispensing method) can be applied. The suitable range of the jetting pressure of the jetted developer and the method of adjusting the jetting pressure of the developer are not particularly limited. For example, ranges and methods disclosed in paragraphs <0631> to <0636> of JP2013-242397A can be used.

In the pattern forming method according to the embodiment of the present invention, a development step (organic solvent developing step) by using a developer containing an organic solvent and a development step (alkali developing step) by using an alkali aqueous solution may be used in combination. Accordingly, a finer pattern can be formed.

According to the present invention, a portion of weak exposure intensity is removed by the organic solvent developing step, but by further performing the alkali developing step, a portion with high exposure intensity is also removed. Since the pattern formation can be performed without dissolving only a region of the intermediate exposure intensity by the multiple development process in which the development is performed a plurality of times in this manner, it is possible to form a pattern finer than usual (the same mechanism as in paragraph <0077> of JP2008-292975A).

After (iii) the development step, it is preferable to include a step (rinsing step) of performing washing with a rinsing solution.

The rinsing solution used in the rinsing step after the step of performing the development with a developer containing an organic solvent is not particularly limited as long as the rinsing solution does not dissolve the resist pattern, and a solution containing a general organic solvent can be used. It is more preferable to use a rinsing solution containing at least one kind of organic solvent selected from the group consisting of a hydrocarbon-based solvent, a ketone-based solvent, an ester-based solvent, an alcohol-based solvent, an amide-based solvent, and an ether-based solvent, as the rinsing solution.

Specific examples of the hydrocarbon-based solvent, the ketone-based solvent, the ester-based solvent, the alcohol-based solvent, the amide-based solvent, and the ether-based solvent include the same solvents as those described for the developer containing an organic solvent.

It is more preferable to perform a washing step by using a rinsing solution containing at least one organic solvent selected from the group consisting of a ketone-based solvent, an ester-based solvent, an alcohol-based solvent, an amide-based solvent, and a hydrocarbon-based solvent after the development step by using a developer containing an organic solvent, it is even more preferable to perform a washing step by using a rinsing solution containing an alcohol-based solvent or an ester-based solvent, it is particularly preferable to perform a washing step by using a rinsing solution containing monohydric alcohol, and it is most preferable to perform a washing step by using a rinsing solution containing monohydric alcohol having 5 or more carbon atoms.

A plurality of components may be mixed or may be mixed with an organic solvent other than the above to be used.

The moisture content in the rinsing solution is preferably 10 mass % or less, more preferably 5 mass % or less, and even more preferably 3 mass % or less. In a case where the moisture content is caused to be 10 mass % or less, satisfactory developing characteristics can be obtained.

An appropriate amount of a surfactant may be added to the rinsing solution to be used.

In the rinsing step, the wafer that has been developed by using a developer containing an organic solvent is subjected to a washing treatment by using the above rinsing solution containing an organic solvent. The method of washing treatment is not particularly limited, and for example, a method of continuously jetting the rinsing solution to the substrate rotating at a constant speed (spin coating method), a method of immersing a substrate in a tank filled with the rinsing solution for a predetermined period of time (dipping method), a method of spraying a rinsing solution to the surface of a substrate (spraying method), and or like can be applied. Among these, it is preferable that a washing treatment is performed by a spin coating method, and after washing, the substrate is rotated at a rotation speed of 2,000 rpm to 4,000 rpm to remove the rinsing solution from the substrate. It is also preferable to include a heating step (post bake) after the rinsing step. The developer and the rinsing solution retained between the patterns and inside the pattern are removed by baking. The heating step after the rinsing step is performed generally at 40° C. to 160° C., and preferably 70° C. to 95° C., and generally for 10 seconds to 3 minutes and preferably 30 seconds to 90 seconds.

The actinic ray-sensitive or radiation-sensitive resin composition according to the embodiment of the present invention and various materials (for example, a resist solvent, a developer, a rinsing solution, an antireflection film forming composition, or a topcoat forming composition) used in the pattern forming method according to the embodiment of the present invention do not preferably include impurities such as metal. The content of impurities included in these materials is preferably 1 ppm or less, more preferably 100 ppt or less, and even more preferably 10 ppt or less, and it is particularly preferable that impurities are not substantially included (the content is equal to or less than a detection limit of a determination device).

Examples of the method for removing impurities such as metals from various materials include filtration using a filter. The pore size of the filter is preferably 10 nm or less, more preferably 5 nm or less, and even more preferably 3 nm or less. The material of the filter is preferably polytetrafluoroethylene, polyethylene, or nylon. As the filter, a filter washed with an organic solvent in advance may be used. In the filter filtration step, a plurality of kinds of filters may be connected in series or juxtaposition, to be used. In a case where a plurality of kinds of filters are used, filters having different pore sizes and/or different materials may be used in combination. In addition, a variety of materials may be used in filtration in a plurality of steps, and the filtration in the plurality of steps may be a circulating filtration step.

Examples of the method for reducing impurities such as metal included in the aforementioned various materials include methods such as selecting raw materials having a less metal content as raw materials for forming various materials, performing filter filtration on raw materials for forming various materials, or performing distillation under conditions where contamination is suppressed as much as possible by lining the inside of a device with TEFLON (registered trademark). Preferable conditions for filter filtration performed on raw materials for forming various materials are the same as the aforementioned conditions.

In addition to the filter filtration, impurities may be removed by an adsorbent, and filter filtration and an adsorbent may be combined to be used. As the adsorbent, well-known adsorbents may be used, and for example, an inorganic adsorbent such as silica gel and zeolite and an organic adsorbent such as activated carbon may be used.

A method of improving the surface roughness of the pattern may be applied to the pattern formed by the pattern forming method according to the embodiment of the present invention. Examples of a method for improving the surface roughness of the pattern include a method of treating a resist pattern by a plasma of gas containing hydrogen disclosed in WO2014/002808A. Well-known methods as disclosed in JP2004-235468A, US2010/0020297A, JP2009-019969A, and Proc. of SPIE Vol. 8328 83280N-1, “EUV Resist Curing Technique for LWR Reduction and Etch Selectivity Enhancement” may be applied.

The pattern forming method according to the embodiment of the present invention can be used in a guide pattern formation (for example, see ACS Nano Vol. 4. No, 8, Pages 4815 to 4823) in Directed Self-Assembly (DSA).

The resist pattern formed, for example, by the above method can be used as a core of a spacer process disclosed in JP1991-270227A (JP-H03-270227A) and JP2013-164509A,

### Method of Manufacturing Electronic Device

The present invention also relates to a method of manufacturing an electronic device including the pattern forming method according to the embodiment of the present invention. The electronic device manufactured by the method of manufacturing the electronic device according to the embodiment of the present invention can be appropriately mounted on electric or electronic apparatuses (for example, household electric devices, office automation (OA)-related apparatuses, or media-related apparatuses, optical apparatuses, and telecommunication apparatuses).

### Examples

Hereinafter, the present invention is specifically described with reference to the examples. A material, an amount used, a proportion, a treatment detail, a treatment order, and the like provided in the following examples can be suitably changed without departing from the gist of the present invention. Accordingly, the scope of the present invention should not be construed in a limited manner by the following examples.

### Preparation of Actinic Ray-Sensitive or Radiation-Sensitive Resin Composition

Hereinafter, various components included in the actinic ray-sensitive or radiation-sensitive resin composition are provided.

### Resin

Structures of the resins (A-1to A-6) presented in Table 2 are provided below.

Weight-average molecular weights (Mw) and dispersion degrees (Mw/Mn) of the resins A-1 to A-6 were measured by GPC (carrier: tetrahydrofuran (THF)) (which were values in terms of polystyrene). The compositional ratio (mol % ratio) of the resin was measured by ¹³C-NMR (nuclear magnetic resonance)

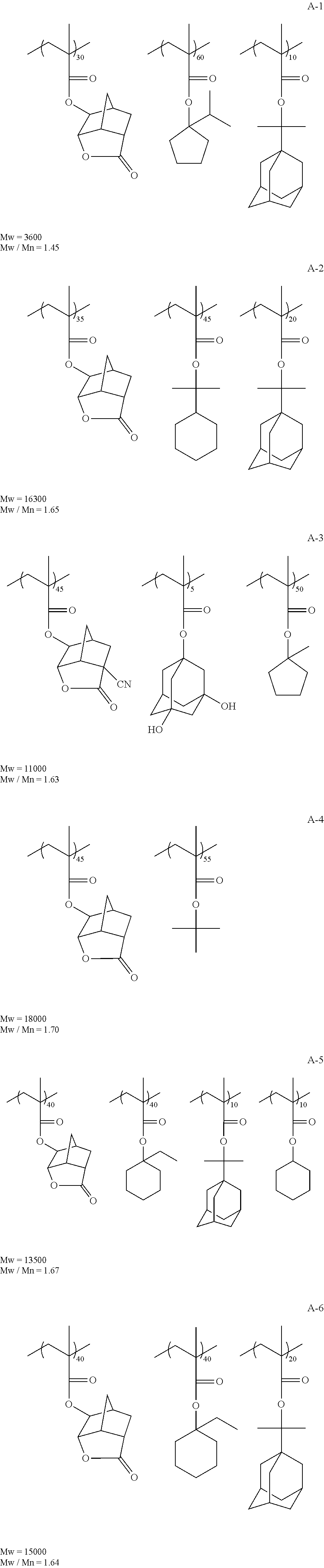

### Compound that Generates Acid Represented by Formula (I) by Irradiation of Actinic Ray or Radiation

Structures of compounds (PAG-1 to PAG-11) that generate an acid represented by Formula (I) due to the irradiation of an actinic ray or radiation which is presented in Table 2 are provided below. PAG-10 and PAG-11 are acid generators for comparison.

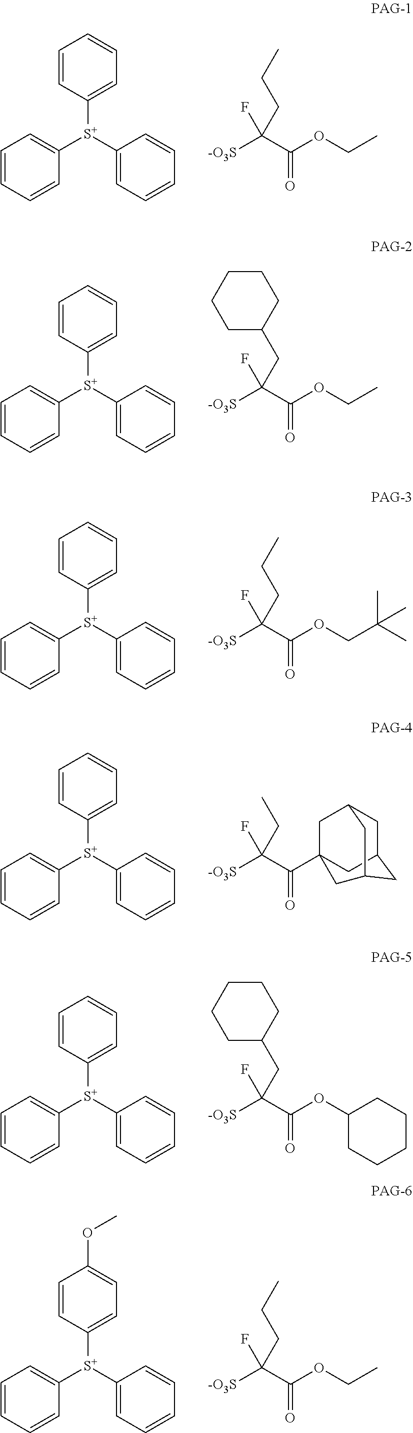

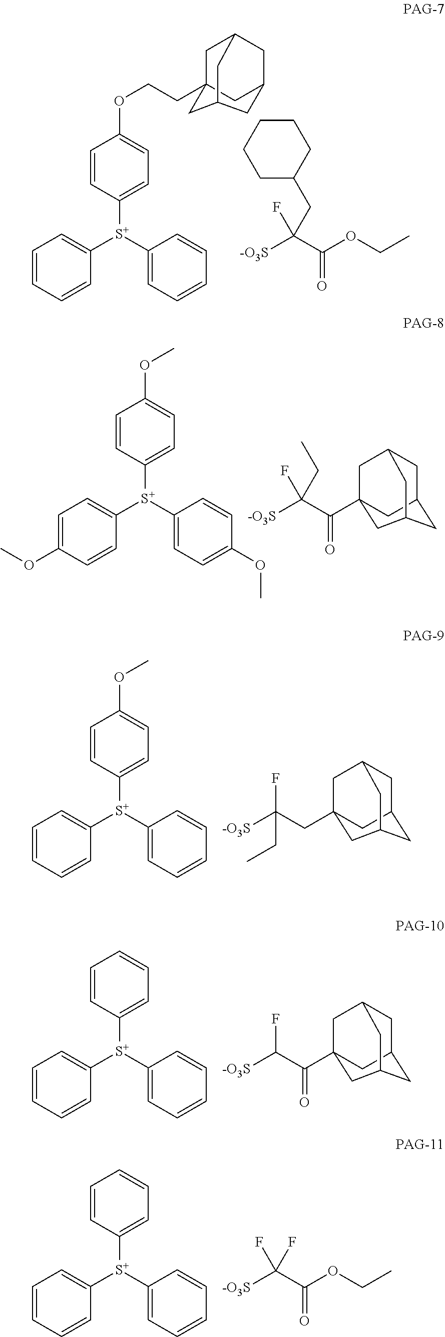

### Synthesis of PAG-1

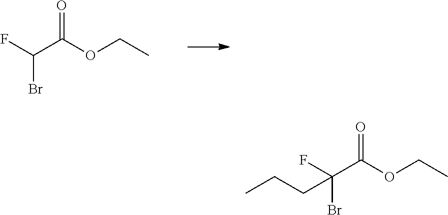

Ethyl bromofluoroacetate (10.0 g) was added to tetrahydrofuran (540 mL) and the obtained solution was cooled to −78° C. Subsequently, lithium diisopropylamide (1.5 mol/L tetrahydrofuran/ethylbenzene/heptane solution: manufactured by Tokyo Chemical Industry Co., Ltd.) (36 mL) was added dropwise to the above solution at −78° C. After completion of the dropwise addition, the obtained reaction solution was stirred for 30 minutes, then iodopropane (13.8 g) was added dropwise to the reaction solution at −78° C. and the temperature was raised to 0° C. The reaction solution was further stirred for four hours, and then a saturated sodium hydrogen carbonate aqueous solution (500 mL) was added. The water phase was extracted 5 times with 100 mL of ethyl acetate, the organic phases were collected and washed with water, and then the solvent was distilled off. The obtained crude product was purified by silica gel chromatography so as to obtain 8.6 g of ethyl 2-bromo-2-fluoropentanoate (yield: 70%).

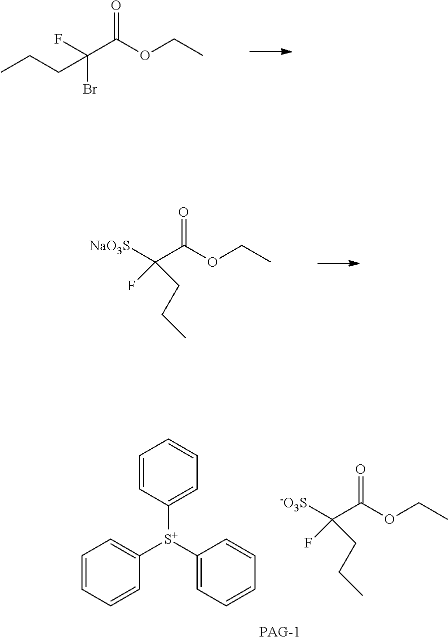

Ethyl 2-bromo-2-fluoropentanoate (5 g) and sodium sulfite (2.7 g) were added to acetonitrile (20 mL) and water (10 mL) and the obtained mixed solution was stirred at 85° C. for six hours. The obtained reaction solution was transferred to a separating funnel, and the water phase was washed twice with hexane. Triphenylsulfonium bromide (7.5 g) and chloroform (20 mL) were added to the obtained aqueous solution, and the mixture was stirred for one hour. Subsequently, the reaction solution was transferred to a separating funnel and the organic phase was washed three times with water (20 mL). The solvent was concentrated with an evaporator so as to obtain 9.7 g of the target compound (PAG-1) as a white solid (yield: 90%).

The same operation as in the synthesis example was performed so as to synthesize PAG-2 to PAG-11.

### Basic Compound

The structures of the basic compounds (N-1 to N-6) presented in Table 2 are provided below.

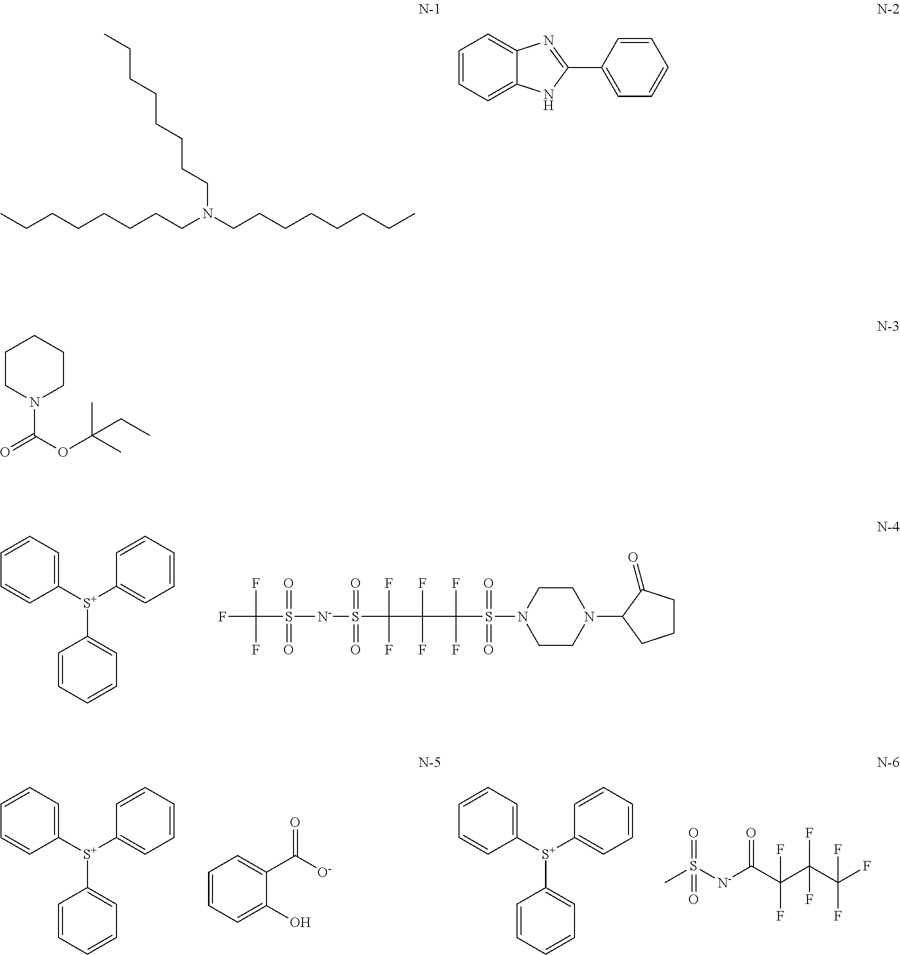

### Hydrophobic Resin

The structures of the hydrophobic resins (1b and 2b) indicated in Table 2 are provided below. In Table 1 below, compositional ratios (molar ratio; corresponding in an order from the left), weight-average molecular weights (Mw), and dispersion degrees (Mw/Mn) of repeating units of the hydrophobic resins 1b and 2b are provided.

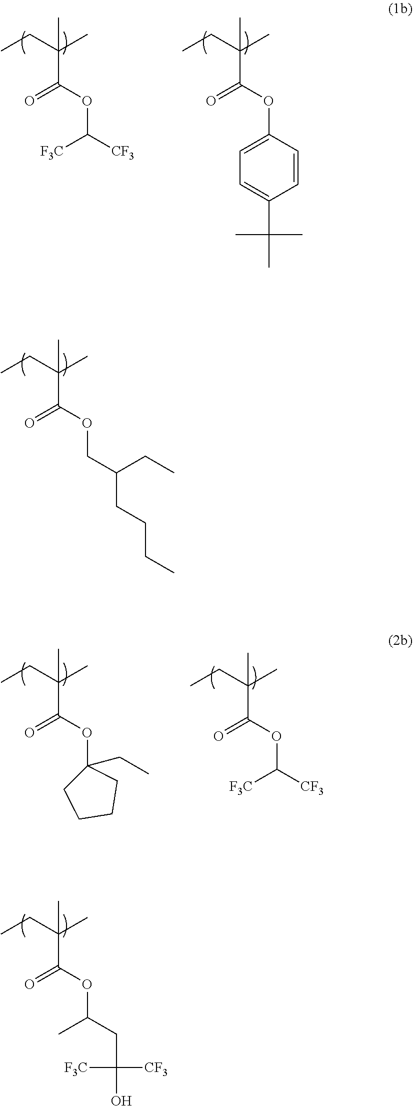

TABLE 1
Compositional ratio, weight-average molecular weight,
and dispersion degree of hydrophobic resins 1b and 2b

| Resin | Compositional ratio | Mw | Mw/Mn |
|---|---|---|---|
| 1b | 50/45/5 | 7000 | 1.3 |
| 2b | 40/40/20 | 18600 | 1.57 |

### Solvent

Solvents presented in Table 2 are as below

SL-1: Propylene glycol monomethyl ether acetate (PGMEA)

SL-2: Propylene glycol monomethyl ether (PGME)

SL-3: Cyclohexanone

SL-4: γ-Butyrolactone

SL-5: Ethyl lactate

### Surfactant

Surfactants presented in Table 2 are as below

W-1: MEGAFACE F176 (manufactured by DIC Corporation; fluorine-based)

W-2: PolyFox PF-6320 (manufactured by OMNOVA Solutions Inc.; fluorine-based)

### Preparation of Actinic Ray-Sensitive or Radiation-Sensitive Resin Composition

The respective components presented in Table 2 were dissolved in the solvents presented in Table 2, so as to respectively prepare solutions having 3.8 mass % of concentration of solid contents. Subsequently, the obtained solution was filtrated through a polyethylene filter having a pore size of 0.1 μm so as to prepare an actinic ray-sensitive or radiation-sensitive resin composition (resist composition).

Subsequently, the obtained resist compositions were evaluated by the following method. Results are as presented in Table 2.

A ratio in a case where a plurality of components were used in Table 2 is a mass ratio.

### Evaluation

### (1) Evaluation of Resist Using Negative Developer

### Forming of Resist Pattern

### ArF Immersion Exposure

A silicon wafer was coated with an organic antireflection film forming composition ARC29SR (manufactured by Nissan Chemical Corporation) and was baked at 205° C. for 60 seconds so as to form an antireflection film having a film thickness of 95 nm. The obtained antireflection film was coated with the resist composition, baking (PB: prebake) was performed at 100° C. for 60 seconds so as to form a resist film having a film thickness of 85 nm.

The obtained wafer was exposed with an ArF excimer laser immersion scanner (manufactured by ASML Netherlands B.V.; XT1700i, NA 1.20, C-Quad, outer sigma 0.900, inner sigma 0.812, XY deflection) through a 6% half tone mask with a 1:1 line and space pattern having a line width of 44 nm. As the immersion liquid, ultrapure water was used. Thereafter, heating (PEB: Post Exposure Bake) was performed at 105° C. for 60 seconds. Subsequently, puddling was performed with a negative developer (butyl acetate) for 30 seconds for development, and puddling was performed with a rinsing solution [methyl isobutyl carbinol (MIBC)] for 30 seconds for rinse. Subsequently, the wafer was rotated at the rotation speed of 4,000 rpm for 30 seconds so as to form a 1:1 line and space pattern with a line width of 44 nm.

### Evaluation of Line Width Roughness (LWR)

The obtained 1:1 line and space pattern having a line width of 44 nm was observed from the top of the pattern with a length-measuring scanning electron microscope (SEM (manufactured by Hitachi, Ltd., S-8840)), the line width was measured at 50 points in the edge range of 2 μm in the longitudinal direction of the line pattern, the standard deviation of the measurement unevenness thereof was obtained so as to calculate 3σ. As the value is smaller, the performance is better. The value of LWR is preferably 3.70 nm or less and more preferably 3.41 nm or less.

### Evaluation of Preservation Stability (Sensitivity Change)

Preservation stability (sensitivity change) was evaluated based on Expression (1) using the exposure amount (mJ/cm²) in a case where a resist pattern of the 1:1 line and space pattern having the line width of 44 nm was formed as an optimum exposure amount.

A smaller numerical value of the optimum exposure amount means that sensitivity is high. As the value (S1/S2) represented by Expression (1) is closer to 1, the value means that sensitivity change is smaller, that is, the preservation stability (sensitivity change) is excellent. The value of the value (S1/S2) represented by Expression (1) is preferably 0.80 or greater and more preferably greater than 0.91.

(Sensitivity change)=(optimal exposure amount S1 in the case of using the resist composition immediately after preparation)/optimal exposure amount S2 in the case of using the resist composition left for one week at 4° C. after preparation) Expression (1)

### (2) Evaluation of Resist Composition

### Evaluation of Preservation Stability (Number of Increased Particles)

First, the number (initial value of the number of particles (pieces/mL)) of particles having a particle diameter of 0.25 μm or more in 1 mL of the resist composition immediately after preparation was measured with a particle counter manufactured by Rion Corporation. Subsequently, the number of particles having a particle diameter of 0.25 μm or more (number of particles (pieces/mL) after elapse of time) in the resist composition left for 3 months at 4° C. after preparation was measured by the same method. Then, based on Expression (2), the number of increased particles was calculated and preservation stability (number of increased particle) was evaluated according to the evaluation standard described below.

(Number of increased particles (pieces/mL))=(Number of particles after elapse of time (pieces/mL))−(Initial value of number of particles(pieces/mL)) Expression (2)

### Evaluation Standard

“A”: The number of increased particles is 0.2 pieces/mL or less

“B”: The number of increased particles is more than 0.2 pieces/mL and 1 piece/mL or less

“C”: The number of increased particles is more than 1 pieces/mL

### (3) Evaluation Result

Results of the above evaluation test are as presented in Table 2.

TABLE 2

| | | | | | | | Evaluation result | | |
|---|---|---|---|---|---|---|---|---|---|
| | Actinic ray-sensitive or radiation-sensitive resin composition (Resist composition) | | | | | | | Preservation stability | |
| | | Acid | Basic | Hydrophobic | | | | | The number |
| | Resin | generator | compound | resin | Surfactant | Solvent | LWR | Sensitivity | of increased |
| | (content (g)) | (content (g)) | (content (g)) | (content (g)) | (content (g)) | (mass ratio) | (nm) | change | particles |
| Example 1 | A-1 (10 g) | PAG-1 (2.0 g) | N-2 (0.31 g) | 1b (0.05 g) | — | SL-1/SL-2 (80/20) | 3.28 | 0.94 | A |
| Example 2 | A-6 (10 g) | PAG-2 (2.0 g) | N-2 (0.29 g) | 1b (0.05 g) | — | SL-1/SL-2 (90/10) | 3.31 | 0.94 | A |
| Example 3 | A-1 (10 g) | PAG-3 (2.0 g) | N-2 (0.29 g) | 1b (0.05 g) | — | SL-1/SL-2 (80/20) | 3.28 | 0.95 | A |
| Example 4 | A-1 (10 g) | PAG-4 (2.1 g) | N-4 (0.30 g) | 1b (0.05 g) | — | SL-1/SL-2 (75/25) | 3.45 | 0.9 | A |
| Example 5 | A-2 (10 g) | PAG-5 (2.1 g) | N-4 (0.30 g) | 1b (0.05 g) | — | SL-1/SL-2 (80/20) | 3.42 | 0.91 | A |
| Example 6 | A-5 (10 g) | PAG-6 (2.0 g) | N-2 (0.31 g) | 1b (0.05 g) | — | SL-1/SL-3 (80/20) | 3.3 | 0.93 | A |
| Example 7 | A-5 (10 g) | PAG-7 (2.5 g) | N-3 (0.30 g) | 1b (0.05 g) | — | SL-1/SL-2 (80/20) | 3.27 | 0.95 | A |
| Example 8 | A-4 (10 g) | PAG-8 (2.0 g) | N-4 (0.30 g) | 1b (0.05 g) | — | SL-1 | 3.46 | 0.91 | A |
| Example 9 | A-2 (10 g) | PAG-9 (1.9 g) | N-2 (0.30 g) | 1b (0.05 g) | — | SL-1/SL-2 (80/20) | 3.55 | 0.94 | A |
| Example 10 | A-3 (10 g) | PAG-1 (2.0 g) | N-2 (0.31 g) | 1b (0.05 g) | — | SL-1/SL-4 (80/20) | 3.3 | 0.95 | A |
| Example 11 | A-6 (10 g) | PAG-2 (2.0 g) | N-2 (0.29 g) | 2b (0.05 g) | — | SL-1/SL-2 (90/10) | 3.35 | 0.93 | A |
| Example 12 | A-3 (10 g) | PAG-1 (2.0 g) | N-2 (0.31 g) | 1b (0.05 g) | W-1 (0.03 g) | SL-1/SL-4 (90/10) | 3.31 | 0.94 | A |
| Example 13 | A-1/A-2 | PAG-4 (2.1 g) | N-4 (0.30 g) | 1b (0.05 g) | — | SL-1/SL-2 (75/25) | 3.42 | 0.91 | A |
| | (5 g/5 g) | | | | | | | | |
| Example 14 | A-5 (10 g) | PAG-7 (2.5 g) | N-5 (0.35 g) | 1b (0.05 g) | — | SL-1/SL-2 (80/20) | 3.25 | 0.95 | A |
| Example 15 | A-5 (10 g) | PAG-6 (2.0 g) | N-6 (0.31 g) | 1b (0.05 g) | — | SL-1/SL-3 (80/20) | 3.29 | 0.94 | A |
| Example 16 | A-1 (10 g) | PAG-1 (2.0 g) | N-2 (0.31 g) | 1b/2b | — | SL-1/SL-2 (80/20) | 3.29 | 0.93 | A |
| | | | | (0.03/0.02 g) | | | | | |
| Example 17 | A-6 (10 g) | PAG-2 (2.0 g) | N-2 (0.29 g) | 1b (0.05 g) | — | SL-1/SL-2/SL-5 | 3.33 | 0.95 | A |
| | | | | | | (20/20/60) | | | |
| Example 18 | A-1 (10 g) | PAG-1 (2.0 g) | N-2/N-4 | 1b (0.05 g) | — | SL-1/SL-2 (80/20) | 3.27 | 0.95 | A |
| | | | (0.15 g/17 g) | | | | | | |
| Example 19 | A-5 (10 g) | PAG-7 (2.5 g) | N-2/N-6 | 1b (0.05 g) | — | SL-1/SL-2 (80/20) | 3.36 | 0.94 | A |
| | | | (0.13 g /0.20 g) | | | | | | |
| Example 20 | A-1/A-6 | PAG-2 (2.0 g) | N-2 (0.29 g) | 2b (0.05 g) | — | SL-1/SL-2 (90/10) | 3.35 | 0.94 | A |
| | (5 g/5 g) | | | | | | | | |
| Example 21 | A-5 (10 g) | PAG-1/PAG-6 | N-2 (0.31 g) | 1b (0.05 g) | — | SL-1/SL-3 (80/20) | 3.3 | 0.93 | A |
| | | (0.9 g/1.3 g) | | | | | | | |
| Example 22 | A-6 (10 g) | PAG-2 (2.0 g) | N-2 (0.29 g) | 1b (0.05 g) | W-2 (0.03 g) | SL-1/SL-2 (90/10) | 3.34 | 0.92 | A |
| Example 23 | A-6 (10 g) | PAG-2 (2.1 g) | N-1 (0.31 g) | 1b (0.05 g) | — | SL-1/SL-2 (90/10) | 3.33 | 0.92 | A |
| Comparative | A-1 (10 g) | PAG-10 (2.0 g) | N-2 (0.31 g) | 1b (0.05 g) | — | SL-1/SL-2 (80/20) | 3.72 | 0.74 | C |
| Example 1 | | | | | | | | | |
| Comparative | A-1 (10 g) | PAG-11 (1.8 g) | N-2 (0.31 g) | 1b (0.05 g) | — | SL-1/SL-2 (80/20) | 3.74 | 0.75 | C |
| Example 2 | | | | | | | | | |

From the results of Table 2, it was confirmed that the resist patterns manufactured by using the actinic ray-sensitive or radiation-sensitive resin compositions of the examples each containing a compound that generates an acid represented by Formula (I) due to the irradiation of the actinic ray or radiation had small LWR.

It was confirmed that the actinic ray-sensitive or radiation-sensitive resin compositions of the examples had excellent preservation stability. That is, it was understood that the composition after elapse of time had a smaller number of increased particles than that in the composition immediately after the preparation, and had substantially the same sensitivity as the composition immediately after the preparation.

From the comparison of Examples 1 to 5, it was confirmed that, in a case where R¹ is a linear or branched alkyl group in the compound that generates an acid represented by Formula (I) due to the irradiation of an actinic ray or radiation, LWR was smaller, and sensitivity decrease after temporal preservation was less.

From the comparison between Examples 4 and 9, in a case where n is 1 (that is, in a case of having electron withdrawing group) in the compound that venerates an acid represented by Formula (I) due to the irradiation of an actinic ray or radiation, LWR was smaller.

Meanwhile, it was clear that the actinic ray-sensitive or radiation-sensitive resin compositions of the comparative examples did not satisfy desired requirements.

What is claimed is:

1. An actinic ray-sensitive or radiation-sensitive resin composition comprising:
a compound that generates an acid represented by Formula (I) by irradiation with an actinic ray or radiation; and
a resin, 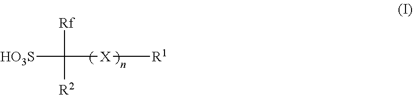
in Formula (I),
R¹ represents an organic group having 1 or more carbon atoms,
R² represents an organic group having 2 or more carbon atoms,
Rf represents a fluorine atom or a monovalent organic group including a fluorine atom,
X represents a divalent electron withdrawing group, and
n represents 0 or 1.

2. The actinic ray-sensitive or radiation-sensitive resin composition according to claim 1,
wherein, in Formula (I), R¹ represents a hydrocarbon group having 1 to 20 carbon atoms.

3. The actinic ray-sensitive or radiation-sensitive resin composition according to claim 1,
wherein, in Formula (I), R² represents a hydrocarbon group having 2 to 20 carbon atoms which may include a hetero atom.

4. The actinic ray-sensitive or radiation-sensitive resin composition according to claim 1,
wherein, in Formula (I), R¹ is a linear or branched alkyl group, and R² is an alkyl group having 2 to 20 carbon atoms.

5. The actinic ray-sensitive or radiation-sensitive resin composition according to claim 1,
wherein, in Formula (I), n is 1.

6. The actinic ray-sensitive or radiation-sensitive resin composition according to claim 1,
wherein the resin is a resin that is decomposed due to an action of an acid to increase polarity.

7. A resist film that is formed of the actinic ray-sensitive or radiation-sensitive resin composition according to claim 1.

8. A pattern forming method comprising:
forming a resist film by using the actinic ray-sensitive or radiation-sensitive resin composition according to claim 1;
exposing the resist film; and
developing the exposed resist film with a developer.

9. The pattern forming method according to claim 8,
wherein the developer contains an organic solvent.

10. A method of manufacturing an electronic device, comprising:
the pattern forming method according to claim 8.
